SEQUENCE LISTING (22 sequences)

SEQ ID NO: 1 - DNA, 1714 bp, Escherichia coli
ctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaataatggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtcgaccggctgcgcggcttcggcggcgaagtgctgctccacggcgcgaactttgatgaagcgaaacgcaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgcttgcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtagcggtaaaacgcatcggtgacgaaaccttccgtttatgccaggagtatctcgacgacatcatcaccgtcgatagcgatgcgatctgtgcggcgatgaaggatttattcgaagatgtgcgcgcggtggcggaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactggtcgaacagcgtgaagcgttgttggcggtgaccattccggaagaaaaaggcagcttcctcaaattctgccaactgcttggcgggcgttcggtcaccgagttcaactaccgttttgccgatgccaaaaacgcctgcatctttgtcggtgtgcgcctgagccgcggcctcgaagagcgcaaagaaattttgcagatgctcaacgacggcggctacagcgtggttgatctctccgacgacgaaatggcgaagctacacgtgcgctatatggtcggcggacgtccatcgcatccgttgcaggaacgcctctacagcttcgaattcccggaatcaccgggcgcgctgctgcgcttcctcaacacgctgggtacgtactggaacatttctttgttccactatcgcagccatggcaccgactacgggcgcgtactggcggcgttcgaacttggcgaccatgaaccggatttcgaaacccggctgaatgagctgggctacgattgccacgacgaaaccaataacccggcgttcaggttctttttggcgggttagggaaaaatgcctgatagcgcttccgttatcaggcct

SEQ ID NO: 2 - protein, 514 aa, Escherichia coli
MADSQPLSGAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKALIVMPTATADIKVDRLRGFGGEVLLHGANFDEAKRKAIELSQQQGFTWVPPFDHPMVIAGQGTLALELLQQDAHLDRVFVPVGGGGLAACVAVLIKQLMPQIKVIAVEAEDSACLKAALDAGHPVDLPRVGLFAEGVAVKRIGDETFRLCQEYLDDIITVDSDAICAAMKDLFEDVRAVAEPSGALALAGMKKYIALHNIRGERLAHILSGANVNFHGLRYVSERCELVEQREALLAVTIPEEKGSFLKFCQLLGGRSVTEFNYRFADAKNACIFVGVRLSRGLEERKEILQMLNDGGYSVVDLSDDEMAKLHVRYMVGGRPSHPLQERLYSFEFPESPGALLRFLNTLGTYWNISLFHYRSHGTDYGRVLAAFELGDHEPDFETRLNELGYDCHDETNNPAFRFFLAG

SEQ ID NO: 3 - protein, 514 aa, Escherichia coli
MADSQPLSGAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKALIVMPTATADIKVDRLRGFGGEVLLHGANFDEAKRKAIELSQQQGFTWVPPFDHPMVIAGQGTLALELLQQDAHLDRVFVPVGGGGLAACVAVLIKQLMPQIKVIAVEAEDSACLKAALDAGHPVDLPRVGLFAEGVAVKRIGDETFRLCQEYLDDIITVDSDAICAAMKDLFEDVRAVAEPSGALALAGMKKYIALHNIRGERLAHILSGANVNFHGLRYVSERCELVEQREALLAVTIPEEKGSFLKFCQLLGGRSVTEFNYRFADAKNACIFVGVRLSRGLEERKEILQMLNDGGYSVVDLSDDEMAKLHVRYMVGGRPSHPLQERLYSFEFPESPGAFLRFLNTLGTYWNISLFHYRSHGTDYGRVLAAFELGDHEPDFETRLNELGYDCHDETNNPAFRFFLAG

SEQ ID NO: 4 - protein, 514 aa, Escherichia coli
MADSQPLSGAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKALIVMPTATADIKVDRLRGFGGEVLLHGANFDEAKRKAIELSQQQGFTWVPPFDHPMVIAGQGTLALELLQQDAHLDRVFVPVGGGGLAACVAVLIKQLMPQIKVIAVEAEDSACLKAALDAGHPVDLPRVGLFAEGVAVKRIGDETFRLCQEYLDDIITVDSDAICAAMKDLFEDVRAVAEPSGALALAGMKKYIALHNIRGERLAHILSGANVNFHGLRYVSERCELVEQREALLAVTIPEEKGSFLKFCQLLGGRSVTEFNYRFADAKNACIFVGVRLSRGLEERKEILQMLNDGGYSVVDLSDDEMAKLHVRYMVGGRPSHPLQERLYSFEFPESPGALLRFLNTLGTYWNISLFHYRSHGTDYGRVLAAFEFGDHEPDFETRLNELGYDCHDETNNPAFRFFLAG

SEQ ID NO: 5 - protein, 514 aa, Escherichia coli
MADSQPLSGAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKALIVMPTATADIKVDRLRGFGGEVLLHGANFDEAKRKAIELSQQQGFTWVPPFDHPMVIAGQGTLALELLQQDAHLDRVFVPVGGGGLAACVAVLIKQLMPQIKVIAVEAEDSACLKAALDAGHPVDLPRVGLFAEGVAVKRIGDETFRLCQEYLDDIITVDSDAICAAMKDLFEDVRAVAEPSGALALAGMKKYIALHNIRGERLAHILSGANVNFHGLRYVSERCELVEQREALLAVTIPEEKGSFLKFCQLLGGRSVTEFNYRFADAKNACIFVGVRLSRGLEERKEILQMLNDGGYSVVDLSDDEMAKLHVRYMVGGRPSHPLQERLYSFEFPESPGALLRFLNTLGTYWNISLFHYRSHGTDYGRVLAAFEYGDHEPDFETRLNELGYDCHDETNNPAFRFFLAG

SEQ ID NO: 6 - protein, 514 aa, Escherichia coli
MADSQPLSGAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKALIVMPTATADIKVDRLRGFGGEVLLHGANFDEAKRKAIELSQQQGFTWVPPFDHPMVIAGQGTLALELLQQDAHLDRVFVPVGGGGLAACVAVLIKQLMPQIKVIAVEAEDSACLKAALDAGHPVDLPRVGLFAEGVAVKRIGDETFRLCQEYLDDIITVDSDAICAAMKDLFEDVRAVAEPSGALALAGMKKYIALHNIRGERLAHILSGANVNFHGLRYVSERCELVEQREALLAVTIPEEKGSFLKFCQLLGGRSVTEFNYRFADAKNACIFVGVRLSRGLEERKEILQMLNDGGYSVVDLSDDEMAKLHVRYMVGGRPSHPLQERLYSFEFPESPGALLRFLNTLGTYWNISLFHYRSHGTDYGRVLAAFEPGDHEPDFETRLNELGYDCHDETNNPAFRFFLAG

SEQ ID NO: 7 - protein, 514 aa, Escherichia coli
MADSQPLSGAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKALIVMPTATADIKVDRLRGFGGEVLLHGANFDEAKRKAIELSQQQGFTWVPPFDHPMVIAGQGTLALELLQQDAHLDRVFVPVGGGGLAACVAVLIKQLMPQIKVIAVEAEDSACLKAALDAGHPVDLPRVGLFAEGVAVKRIGDETFRLCQEYLDDIITVDSDAICAAMKDLFEDVRAVAEPSGALALAGMKKYIALHNIRGERLAHILSGANVNFHGLRYVSERCELVEQREALLAVTIPEEKGSFLKFCQLLGGRSVTEFNYRFADAKNACIFVGVRLSRGLEERKEILQMLNDGGYSVVDLSDDEMAKLHVRYMVGGRPSHPLQERLYSFEFPESPGALLRFLNTLGTYWNISLFHYRSHGTDYGRVLAAFEEGDHEPDFETRLNELGYDCHDETNNPAFRFFLAG

SEQ ID NO: 8 - protein, 514 aa, Escherichia coli
MADSQPLSGAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKALIVMPTATADIKVDRLRGFGGEVLLHGANFDEAKRKAIELSQQQGFTWVPPFDHPMVIAGQGTLALELLQQDAHLDRVFVPVGGGGLAACVAVLIKQLMPQIKVIAVEAEDSACLKAALDAGHPVDLPRVGLFAEGVAVKRIGDETFRLCQEYLDDIITVDSDAICAAMKDLFEDVRAVAEPSGALALAGMKKYIALHNIRGERLAHILSGANVNFHGLRYVSERCELVEQREALLAVTIPEEKGSFLKFCQLLGGRSVTEFNYRFADAKNACIFVGVRLSRGLEERKEILQMLNDGGYSVVDLSDDEMAKLHVRYMVGGRPSHPLQERLYSFEFPESPGALLRFLNTLGTYWNISLFHYRSHGTDYGRVLAAFETGDHEPDFETRLNELGYDCHDETNNPAFRFFLAG

SEQ ID NO: 9 - protein, 514 aa, Escherichia coli
MADSQPLSGAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKALIVMPTATADIKVDRLRGFGGEVLLHGANFDEAKRKAIELSQQQGFTWVPPFDHPMVIAGQGTLALELLQQDAHLDRVFVPVGGGGLAACVAVLIKQLMPQIKVIAVEAEDSACLKAALDAGHPVDLPRVGLFAEGVAVKRIGDETFRLCQEYLDDIITVDSDAICAAMKDLFEDVRAVAEPSGALALAGMKKYIALHNIRGERLAHILSGANVNFHGLRYVSERCELVEQREALLAVTIPEEKGSFLKFCQLLGGRSVTEFNYRFADAKNACIFVGVRLSRGLEERKEILQMLNDGGYSVVDLSDDEMAKLHVRYMVGGRPSHPLQERLYSFEFPESPGALLRFLNTLGTYWNISLFHYRSHGTDYGRVLAAFEQGDHEPDFETRLNELGYDCHDETNNPAFRFFLAG

SEQ ID NO: 10 - protein, 514 aa, Escherichia coli
MADSQPLSGAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKALIVMPTATADIKVDRLRGFGGEVLLHGANFDEAKRKAIELSQQQGFTWVPPFDHPMVIAGQGTLALELLQQDAHLDRVFVPVGGGGLAACVAVLIKQLMPQIKVIAVEAEDSACLKAALDAGHPVDLPRVGLFAEGVAVKRIGDETFRLCQEYLDDIITVDSDAICAAMKDLFEDVRAVAEPSGALALAGMKKYIALHNIRGERLAHILSGANVNFHGLRYVSERCELVEQREALLAVTIPEEKGSFLKFCQLLGGRSVTEFNYRFADAKNACIFVGVRLSRGLEERKEILQMLNDGGYSVVDLSDDEMAKLHVRYMVGGRPSHPLQERLYSFEFPESPGALLRFLNTLGTYWNISLFHYRSHGTDYGRVLAAFEIGDHEPDFETRLNELGYDCHDETNNPAFRFFLAG

SEQ ID NO: 11 - protein, 514 aa, Escherichia coli
MADSQPLSGAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKALIVMPTATADIKVDRLRGFGGEVLLHGANFDEAKRKAIELSQQQGFTWVPPFDHPMVIAGQGTLALELLQQDAHLDRVFVPVGGGGLAACVAVLIKQLMPQIKVIAVEAEDSACLKAALDAGHPVDLPRVGLFAEGVAVKRIGDETFRLCQEYLDDIITVDSDAICAAMKDLFEDVRAVAEPSGALALAGMKKYIALHNIRGERLAHILSGANVNFHGLRYVSERCELVEQREALLAVTIPEEKGSFLKFCQLLGGRSVTEFNYRFADAKNACIFVGVRLSRGLEERKEILQMLNDGGYSVVDLSDDEMAKLHVRYMVGGRPSHPLQERLYSFEFPESPGALLRFLNTLGTYWNISLFHYRSHGTDYGRVLAAFEVGDHEPDFETRLNELGYDCHDETNNPAFRFFLAG

SEQ ID NO: 12 - protein, 514 aa, Escherichia coli
MADSQPLSGAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKALIVMPTATADIKVDRLRGFGGEVLLHGANFDEAKRKAIELSQQQGFTWVPPFDHPMVIAGQGTLALELLQQDAHLDRVFVPVGGGGLAACVAVLIKQLMPQIKVIAVEAEDSACLKAALDAGHPVDLPRVGLFAEGVAVKRIGDETFRLCQEYLDDIITVDSDAICAAMKDLFEDVRAVAEPSGALALAGMKKYIALHNIRGERLAHILSGANVNFHGLRYVSERCELVEQREALLAVTIPEEKGSFLKFCQLLGGRSVTEFNYRFADAKNACIFVGVRLSRGLEERKEILQMLNDGGYSVVDLSDDEMAKLHVRYMVGGRPSHPLQERLYSFEFPESPGALLRFLNTLGTYWNISLFHYRSHGTDYGRVLAAFEMGDHEPDFETRLNELGYDCHDETNNPAFRFFLAG

SEQ ID NO: 13 - protein, 514 aa, Escherichia coli
MADSQPLSGAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKALIVMPTATADIKVDRLRGFGGEVLLHGANFDEAKRKAIELSQQQGFTWVPPFDHPMVIAGQGTLALELLQQDAHLDRVFVPVGGGGLAACVAVLIKQLMPQIKVIAVEAEDSACLKAALDAGHPVDLPRVGLFAEGVAVKRIGDETFRLCQEYLDDIITVDSDAICAAMKDLFEDVRAVAEPSGALALAGMKKYIALHNIRGERLAHILSGANVNFHGLRYVSERCELVEQREALLAVTIPEEKGSFLKFCQLLGGRSVTEFNYRFADAKNACIFVGVRLSRGLEERKEILQMLNDGGYSVVDLSDDEMAKLHVRYMVGGRPSHPLQERLYSFEFPESPGALLRFLNTLGTYWNISLFHYRSHGTDYGRVLAAFEKGDHEPDFETRLNELGYDCHDETNNPAFRFFLAG

SEQ ID NO: 14 - DNA, 1544 bp, Artificial
  variant allele
atggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtcgaccggctgcgcggcttcggcggcgaagtgctgctccacggcgcgaactttgatgaagcgaaacgcaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgcttgcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtagcggtaaaacgcatcggtgacgaaaccttccgtttatgccaggagtatctcgacgacatcatcaccgtcgatagcgatgcgatctgtgcggcgatgaaggatttattcgaagatgtgcgcgcggtggcggaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactggtcgaacagcgtgaagcgttgttggcggtgaccattccggaagaaaaaggcagcttcctcaaattctgccaactgcttggcgggcgttcggtcaccgagttcaactaccgttttgccgatgccaaaaacgcctgcatctttgtcggtgtgcgcctgagccgcggcctcgaagagcgcaaagaaattttgcagatgctcaacgacggcggctacagcgtggttgatctctccgacgacgaaatggcgaagctacacgtgcgctatatggtcggcggacgtccatcgcatccgttgcaggaacgcctctacagcttcgagttcccggaatcaccgggcgcgttcctgcgcttcctcaacacgctgggtacgtactggaacatttctttgttccactatcgcagccatggcaccgactacgggcgcgtactggcggcgttcgaacttggcgaccatgaaccggatttcgaaacccggctgaatgagctgggctacgattgccacgacgaaaccaataacccggcgttcaggttctttttggcgggtta

SEQ ID NO: 15 - DNA, 1544 bp, Artificial
  variant allele
atggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtcgaccggctgcgcggcttcggcggcgaagtgctgctccacggcgcgaactttgatgaagcgaaacgcaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgcttgcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtagcggtaaaacgcatcggtgacgaaaccttccgtttatgccaggagtatctcgacgacatcatcaccgtcgatagcgatgcgatctgtgcggcgatgaaggatttattcgaagatgtgcgcgcggtggcggaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactggtcgaacagcgtgaagcgttgttggcggtgaccattccggaagaaaaaggcagcttcctcaaattctgccaactgcttggcgggcgttcggtcaccgagttcaactaccgttttgccgatgccaaaaacgcctgcatctttgtcggtgtgcgcctgagccgcggcctcgaagagcgcaaagaaattttgcagatgctcaacgacggcggctacagcgtggttgatctctccgacgacgaaatggcgaagctacacgtgcgctatatggtcggcggacgtccatcgcatccgttgcaggaacgcctctacagcttcgaattcccggaatcaccgggcgcgctgctgcgcttcctcaacacgctgggtacgtactggaacatttctttgttccactatcgcagccacggcaccgactacgggcgcgtactggcggcgttcgaatttggcgaccatgaaccggatttcgaaacccggctgaatgagctgggctacgattgccacgacgaaaccaataacccggcgttcaggttctttttggcgggtta

SEQ ID NO: 16 - DNA, 1644 bp, Escherichia coli
atggcgcacagtgggtggtacatgcgttgcgggcacagggtgtgaacaccgttttcggttatccgggtggcgcaattatgccggtttacgatgcattgtatgacggcggcgtggagcacttgctatgccgacatgagcagggtgcggcaatggcggctatcggttatgctcgtgctaccggcaaaactggcgtatgtatcgccacgtctggtccgggcgcaaccaacctgataaccgggcttgcggacgcactgttagattccatccctgttgttgccatcaccggtcaagtgtccgcaccgtttatcggcactgacgcatttcaggaagtggatgtcctgggattgtcgttagcctgtaccaagcatagctttctggtgcagtcgctggaagagttgccgcgcatcatggctgaagcattcgacgttgcctgctcaggtcgtcctggtccggttctggtcgatatcccaaaagatatccagttagccagcggtgacctggaaccgtggttcaccaccgttgaaaacgaagtgactttcccacatgccgaagttgagcaagcgcgccagatgctggcaaaagcgcaaaaaccgatgctgtacgttggcggtggcgtgggtatggcgcaggcagttccggctttgcgtgaatttctcgctgccacaaaaatgcctgccacctgtacgctgaaagggctgggcgcagtagaagcagattatccgtactatctgggcatgctggggatgcacggcaccaaagcggcaaacttcgcggtgcaggagtgtgacctgctgatcgccgtgggcgcacgttttgatgaccgggtgaccggcaaactgaacaccttcgcgccacacgccagtgttatccatatggatatcgacccggcagaaatgaacaagctgcgtcaggcacatgtggcattacaaggtgatttaaatgctctgttaccagcattacagcagccgttaaatcaatatgactggcagcaacactgcgcgcagctgcgtgatgaacattcctggcgttacgaccatcccggtgacgctatctacgcgccgttgttgttaaaacaactgtcggatcgtaaacctgcggattgcgtcgtgaccacagatgtggggcagcaccagatgtgggctgcgcagcacatcgcccacactcgcccggaaaatttcatcacctccagcggtttaggtaccatgggttttggtttaccggcggcggttggcgcacaagtcgcgcgaccgaacgataccgttgtctgtatctccggtgacggctctttcatgatgaatgtgcaagagctgggcaccgtaaaacgcaagcagttaccgttgaaaatcgtcttactcgataaccaacggttagggatggttcgacaatggcagcaactgttttttcaggaacgatacagcgaaaccacccttactgataaccccgatttcctcatgttagccagcgccttcggcatccatggccaacacatcacccggaaagaccaggttgaagcggcactcgacaccatgctgaacagtgatgggccatacctgcttcatgtctcaatcgacgaacttgagaacgtctggccgctggtgccgcctggcgccagtaattcagaaatgttggagaaattatcatgag

SEQ ID NO: 17 - DNA, 285 bp, Escherichia coli
tggggaattctcatgatgcaacatcaggtcaatgtatcggctcgcttcaatccagaaaccttagaacgtgttttacgcgtggtgcgtcatcgtggtttccacgtctgctcaatgaatatggccgccgccagcgatgcacaaaatataaatatcgaattgaccgttgccagcccacggtcggtcgacttactgtttagtcagttaaataaactggtggacgtcgcacacgttgccatctgccagagcacaaccacatcacaacaaatccgcgcctgataagaattc

SEQ ID NO: 18 - DNA, 1201 bp, Escherichia coli
atggcgcacagtgggtggtacatgcgttgcgggcacagggtgtgaacaccgttttcggttatccgggtggcgcaattatgccggtttacgatgcattgtatgacggcggcgtggagcacttgctatgccgacatgagcagggtgcggcaatggcggctatcggttatgctcgtgctaccggcaaaactggcgtatgtatcgccacgtctggtccgggcgcaaccaacctgataaccgggcttgcggacgcactgttagattccatccctgttgttgccatcaccggtcaagtgtccgcaccgtttatcggcactgacgcatttcaggaagtggatgtcctgggattgtcgttagcctgtaccaagcatagctttctggtgcagtcgctggaagagttgccgcgcatcatggctgaagcattcgacgttgcctgctcaggtcgtcctggtccggttctggtcgatatcccaaaagatatccagttagccagcggtgacctggaaccgtggttcaccaccgttgaaaacgaagtgactttcccacatgccgaagttgagcaagcgcgccagatgctggcaaaagcgcaaaaaccgatgctgtacgttggcggtggcgtgggtatggcgcaggcagttccggctttgcgtgaatttctcgctgccacaaaaatgcctgccacctgtacgctgaaagggctgggcgcagtagaagcagattatccgtactatctgggcatgctggggatgcacggcaccaaagcggcaaacttcgcggtgcaggagtgtgacctgctgatcgccgtgggcgcacgttttgatgaccgggtgaccggcaaactgaacaccttcgcgccacacgccagtgttatccatatggatatcgacccggcagaaatgaacaagctgcgtcaggcacatgtggcattacaaggtgatttaaatgctctgttaccagcattacagcagccgttaaatcaatatgactggcagcaacactgcgcgcagctgcgtgatgaacattcctggcgttacgaccatcccggtgacgctatctacgcgccgttgttgttaaaacaactgtcggatcgtaaacctgcggattgcgtcgtgaccacagatgtggggcagcaccagatgtgggctgcgcagcacatcgcccacactcgcccggaaaatttcatcacctccagcggtttaggtac

SEQ ID NO: 19 - DNA, 264 bp, Arabidopsis thaliana
atggcttcctctatgctctcttccgctactatggttgcctctccggctcaggccactatggtcgctcctttcaacggacttaagtcctccgctgccttcccagccacccgcaaggctaacaacgacattacttccatcacaagcaacggcggaagagttaactgcatgcaggtgtggcctccgattggaaagaagaagtttgagactctctcttaccttcctgaccttaccgattccggtggtcgcgtcaactgcatgcaggcc

SEQ ID NO: 20 - DNA, 443 bp, Escherichia coli
catgggttttggtttaccggcggcggttggcgcacaagtcgcgcgaccgaacgataccgttgtctgtatctccggtgacggctctttcatgatgaatgtgcaagagctgggcaccgtaaaacgcaagcagttaccgttgaaaatcgtcttactcgataaccaacggttagggatggttcgacaatggcagcaactgttttttcaggaacgatacagcgaaaccacccttactgataaccccgatttcctcatgttagccagcgccttcggcatccatggccaacacatcacccggaaagaccaggttgaagcggcactcgacaccatgctgaacagtgatgggccatacctgcttcatgtctcaatcgacgaacttgagaacgtctggccgctggtgccgcctggcgccagtaattcagaaatgttggagaaattatcatgag

SEQ ID NO: 21 - protein, 514 aa, Artificial [sequence omitted]

SEQ ID NO: 22 - protein, 592 aa, Arabidopsis thaliana
MNSVQLPTAQSSLRSHIHRPSKPVVGFTHFSSRSRIAVAVLSRDETSMTPPPPKLPLPRLKVSPNSLQYPAGYLGAVPERTNEAENGSIAEAMEYLTNILSTKVYDIAIESPLQLAKKLSKRLGVRMYLKREDLQPVFSFKLRGAYNMMVKLPADQLAKGVICSSAGNHAQGVALSASKLGCTAVIVMPVTTPEIKWQAVENLGATVVLFGDSYDQAQAHAKIRAEEEGLTFIPPFDHPDVIAGQGTVGMEITRQAKGPLHAIFVPVGGGGLIAGIAAYVKRVSPEVKIIGVEPADANAMALSLHHGERVILDQVGGFADGVAVKEVGEETFRISRNLMDGVVLVTRDAICASIKDMFEEKRNILEPAGALALAGAEAYCKYYGLKDVNVVAITSGANMNFDKLRIVTELANVGRQQEAVLATLMPEKPGSFKQFCELVGPMNISEFKYRCSSEKEAVVLYSVGVHTAGELKALQKRMESSQLKTVNLTTSDLVKDHLRYLMGGRSTVGDEVLCRFTFPERPGALMNFLDSFSPRWNITLFHYRGQGETGANVLVGIQVPEQEMEEFKNRAKALGYDYFLVSDDDYFKLLMH

(19) United States
(12) Patent Application Publication
(10) Pub. No.: US 2005/0289668 A1
(43) Pub. Date: Dec. 29, 2005

(54) Plants with increased levels of one or more amino acids

(76) Inventors: Lisa M. Weaver, O'Fallon, MO (US); Timothy A. Mitsky, Maryland Heights, MO (US); William D. Rapp, St. Louis, MO (US); Kenneth J. Gruys, Davis, CA (US); Jihong Liang, Chesterfield, MO (US); Gabriela Vaduva, Wildwood, MO (US)

Correspondence Address: FULBRIGHT & JAWORSKI L.L.P., 600 CONGRESS AVE., SUITE 2400, AUSTIN, TX, 78701, US

(21) Appl. No.: 10/840,688

(22) Filed: May 6, 2004

Related U.S. Application Data
(60) Provisional application No. 60/468,727, filed on May 7, 2003.

(51) Int. Cl. A01H 1/00; C12N 15/82; A01H 5/00; C12N 5/04

(52) U.S. Cl. 800/281; 800/312; 435/415; 435/468

(57) ABSTRACT

The present invention provides DNA constructs comprising exogenous polynucleotides encoding a threonine deaminase and/or AHAS. Transgenic plants transformed with the constructs, as well as seed and progeny dervied from these plants, are also provided. The transgenic plants have an increased level of one or more amino acids as compared to a non-transgenic plant of the same species.

This application claims the benefit of U.S. Provisional Application No. 60/468,727, filed May 7, 2003, herein incorporated by reference in its entirety.

The field of the present invention is agricultural biotechnology. More specifically, the present invention relates to biotechnical approaches to increase the level of amino acids in plants.

A number of important crops, including soybean and maize, do not contain sufficient quantities or the correct balance of several amino acids to be nutritionally complete. This is especially true for the branched chained amino acids (BCAA) leucine, isoleucine, and valine. BCAA are essential amino acids since humans are not able to synthesize these molecules and hence must acquire them from their diet. Isoleucine is a branched chain amino acid that is synthesized from threonine. Threonine itself is synthesized from aspartate. The synthetic route between aspartate and BCAA involves several enzymes that are allosterically inhibited by various amino acids. The enzymes used in the synthesis of BCAA include aspartate kinase (AK), bifunctional aspartate kinase—homoserine dehydrogenase (AK-HSDH), isopropylmalate synthase, threonine deaminase (TD), and acetohydroxy acid synthase (AHAS). In particular, threonine deaminase (EC 4.2.1.16) (TD, threonine dehydratase; L-threonine hydrolyase (deaminating)) and acetohydroxyacid synthase (AHAS; acetolactate synnthase (EC 4.1.3.18)) are key enzymes in the biosynthesis of BCAA.

In E. coli, threonine deaminase exists in separate biosynthetic and biodegradative forms. The biosynthetic form of threonine deaminase is encoded by the gene ilvA and catalyzes the first committed step in the biosynthesis of branched chain amino acids in plants and microorganisms. This step dehydrates and deaminates L-threonine to produce 2-oxobutyrate by utilizing pyridoxal 5′-phosphate (PryP). Biosynthetic threonine deaminase is subject to allosteric regulation by L-isoleucine (Umbarger, Science, 123:848 (1956); Umbarger, Protein Science, 1:1392 (1992); Changeux, Cold Spring Harbor Symp. Quant. Biol., 26:313 (1961); Monod et al., J. Mol. Biol., 6:306 (1963)). Several deregulated enzymes of threonine deaminase exist from both plants and bacteria. See, Feldberg et al., Eur. J. Biochem., 21:438-446 (1971); Mourad et al., Plant Phys., 107:43-52 (1995); Fisher et al., J. Bact., 175:6605-6613 (1993); Taillon et al., Gene, 63:245-252 (1988); Möckel et al., Mol. Microbiol., 13:833-842 (1994); Guillouet et al., Appl Environ Microbiol., 65:3100-3107 (1999); Slater et al., Nature Biotechnology, 7:1011-1016 (1999).

In contrast to the biosynthetic form, the biodegradative form of threonine deaminase is activated by AMP, is insensitive to feedback regulation by L-isoleucine, and is produced anaerobically in medium containing high concentrations of amino acids and no glucose. Moreover, in E. coli, the biodegradative form of threonine deaminase is encoded by a separate gene (tdcB).

AHAS enzymes are conserved across a number of organisms such as bacteria, yeast, and plants (Singh et al., Proc. Natl. Acad. Sci., 88:4572-4576 (1991)). In E. coli and other enterobacteria, AHAS is a heterotetramic protein composed of two large and two small subunits, termed ilvG and ilvM, respectively (Weinstock et al., J. Bacteriol., 174:5560-6 (1992)). The enzymatic activity of the tetramer is contained entirely in the large subunit. The small subunit is required for enzyme stability and regulatory purposes. In plants, the aggregation state varies among species. In some plants, such as Arabidopsis thaliana, a single structural gene encodes the AHAS enzyme (Andersson et al., Plant Cell Reports, 22:261-267 (2003)), while in other plant species, such as tobacco, there may be more than one functional gene. Like bacteria, plant AHAS enzymes are also feedback inhibited. Plant AHAS enzymes are the target of some commercial herbicides (U.S. Pat. No. 6,727,414).

AHAS plays an important role in balancing the levels of leucine and valine on the one hand and isoleucine on the other. AHAS is important in driving the conversion of pyruvate to acetolactate, the precursor to both leucine and valine. AHAS also drives the conversion of 2-oxobutyrate to acetohydroxybutyrate, the precursor to isoleucine. Because AHAS has a substrate preference for 2-oxobutyrate over pyruvate the enzymatic reaction favors the production of isoleucine. Isoleucine levels are held in check by the feedback inhibition of TD by isoleucine while AHAS is feedback inhibited by valine and leucine. Leucine production is also regulated by feedback inhibition of isopropylmalate synthase.

BCAA are produced commercially by direct extraction of the amino acid from protein hydrolysates. For example, the current level of isoleucine production is less than 400 metric tons per year but demand for isoleucine is increasing. Therefore, to provide for the shortfall in isolated BCAA, as well as provide a more economic source of it, plants that are engineered to synthesize increased levels of amino acids are needed.

### SUMMARY OF THE INVENTION

The present invention includes a DNA construct comprising multiple plant expression cassettes wherein a first expression cassette comprises a promoter functional in cells of a plant operably linked to an exogenous polynucleotide encoding a feedback insensitive threonine deaminase and a second expression cassette comprises a promoter functional in cells of a plant operably linked to an exogenous polynucleotide encoding AHAS. In one embodiment, the DNA construct of the present invention comprises multiple plant expression cassettes wherein a first expression cassette comprises a promoter functional in cells of a plant operably linked to an exogenous polynucleotide encoding a feedback insensitive threonine deaminase, a second expression cassette comprises a large subunit of AHAS, and a third expression cassette comprises a promoter functional in cells of a plant operably linked to an exogenous polynucleotide encoding a small subunit of AHAS. In one embodiment, each of the promoters is a seed enhanced promoter. In another embodiment, each of the promoters is selected from the group consisting of: napin, 7S alpha, 7S alpha′, 7S beta, USP 88, enhanced USP 88, Arcelin 5, and Oleosin. In one embodiment, there are at least two different seed enhanced promoters.

In one aspect of the present invention, the first cassette comprises a polynucleotide encoding a feedback insensitive threonine deaminase comprising SEQ ID NO: 22. In one embodiment, the polynucleotide is SEQ ID NO: 22. In another aspect of the present invention, the first cassette comprises an exogenous polynucleotide encoding a threonine deaminase variant allele or subunit thereof comprising an amino acid substitution at position L447F, or L481F, or L481Y, or L481P, or L481E, or L481T, or L481Q, or L81I, or L481V, or L481M, or L481K. In yet another aspect of the present invention, the polynucleotide encoding a threonine deaminase variant allele comprises SEQ ID NO: 2. In another aspect of the present invention, the polynucleotide is SEQ ID NO: 2.

In one embodiment of the present invention, the first cassette further comprises a polynucleotide encoding a plastid transit peptide operably linked to polynucleotide encoding the threonine deaminase, threonine deaminase variant allele, or subunit thereof.

In another embodiment, the second expression cassette comprises a polynucleotide encoding the large subunit of AHAS. In one embodiment, the polynucleotide encoding the large subunit of AHAS comprises SEQ ID NO: 16. In one embodiment, the polynucleotide is SEQ ID NO: 16. In still another embodiment, a polynucleotide encoding a plastid transit peptide is operably linked to the polynucleotide encoding the large subunit of AHAS. In one embodiment, the third expression cassette comprises a polynucleotide encoding the small subunit of AHAS. In another embodiment, the polynucleotide encoding the small subunit of AHAS comprises SEQ ID NO: 17. In one embodiment, the polynucleotide is SEQ ID NO: 17. In yet another embodiment, a polynucleotide encoding a plastid transit peptide is operably linked to the polynucleotide encoding the small subunit of AHAS.

In one aspect, a DNA construct comprises multiple plant expression cassettes wherein a first expression cassette comprises a promoter functional in cells of a plant operably linked to an exogenous polynucleotide encoding a feedback insensitive threonine deaminase, and a second expression cassette comprises a promoter functional in cells of a plant operably linked to an exogenous polynucleotide encoding a large subunit of AHAS. In another aspect, each of the promoters is a seed enhanced promoter. In still another aspect, each of the seed enhanced promoters is selected from the group consisting of: napin, 7S alpha, 7S alpha′, 7S beta, USP 88, enhanced USP 88, Arcelin 5, and Oleosin. In another aspect, there are at least two different seed enhanced promoters in the construct.

In one embodiment, the first cassette comprises a polynucleotide encoding a feedback insensitive threonine deaminase comprising SEQ ID NO: 22. In one embodiment, the polynucleotide is SEQ ID NO: 22. In another embodiment, the first cassette comprises a threonine deaminase variant allele comprising an amino acid substitution at position L447F, or L481F, or L481Y, or L481P, or L481E, or L481T, or L481Q, or L481I, or L481V, or L481M, or L481K. In another embodiment, the polynucleotide encoding a threonine deaminase variant allele comprises SEQ ID NO: 2 comprising an amino acid substitution at position L447F, or L481F, or L481Y, or L481P, or L481E, or L481T, or L481Q, or L481I, or L481V, or L481M, or L481K. In one embodiment, the polynucleotide is SEQ ID NO: 22. In one aspect of the present invention, the first cassette comprises a polynucleotide encoding a plastid transit peptide operably linked to said polynucleotide encoding a threonine deaminase. In another aspect, the second expression cassette comprises a polynucleotide encoding the large subunit of AHAS. In yet another aspect, the polynucleotide encoding the large subunit of AHAS comprises SEQ ID NO: 16. In one embodiment, the polynucleotide is SEQ ID NO: 16. In still another aspect, a polynucleotide encoding a plastid transit peptide is operably linked to said polynucleotide encoding said large subunit of AHAS.

In one embodiment, the DNA construct comprises multiple plant expression cassettes wherein an expression cassette comprising a promoter functional in cells of a plant is operably linked to an exogenous polynucleotide encoding a monomeric AHAS. In another embodiment, the DNA construct comprises multiple plant expression cassettes wherein a first expression cassette comprising a promoter functional in cells of a plant is operably linked to an exogenous polynucleotide encoding a large subunit of AHAS, and a second expression cassette comprising a promoter functional in cells of a plant is operably linked to an exogenous polynucleotide encoding a small subunit of AHAS. In still another embodiment, each of the promoters is a seed enhanced promoter. In yet another embodiment, each of said seed enhanced promoters is selected from the group consisting of: napin, 7S alpha, 7S alpha′, 7S beta, USP 88, enhanced USP 88, Arcelin 5, and Oleosin. In another embodiment, there are at least two different seed enhanced promoters. In one embodiment, the first cassette comprises a large subunit of AHAS comprising SEQ ID NO: 16. In one embodiment, the polynucleotide is SEQ ID NO: 16. In another embodiment, the first cassette comprises a polynucleotide encoding a plastid transit peptide operably linked to said polynucleotide encoding said large subunit of AHAS. In another embodiment, the second cassette comprises a polynucleotide encoding the small subunit of AHAS. In another embodiment, the second cassette comprises a polynucleotide encoding the small subunit of AHAS comprising SEQ ID NO: 17. In one embodiment, the polynucleotide is SEQ ID NO: 17. In another embodiment, the second cassette comprises a polynucleotide encoding a plastid transit peptide operably linked to said polynucleotide encoding said small subunit of AHAS.

The present invention also provides a method for preparing a transgenic dicot plant having an increase in amino acid level in the seed as compared to a seed from a non-transgenic plant of the same plant species, comprising the steps of: a) introducing into regenerable cells of a dicot plant a transgene comprising a construct comprising a polynucleotide encoding a feedback insensitive threonine deaminase; b) regenerating said regenerable cell into a dicot plant; c) harvesting seed from said plant; d) selecting one or more seeds with an increased level of amino acid as compared to a seed from a non-trangenic plant of the same plant species; and e) planting said seed, wherein, if isoleucine is present at an increased level, at least one additional level of amino acid is also increased. In one embodiment, the dicot plant is a soybean plant. In one embodiment, the increased level of amino acids comprises an increase in the concentration of: a) fle and one or more of Arg, Asn, Asp, His, Met, Ala, Leu, Thr, Val, Gln, Tyr, Lys, Ser, and Phe; or b) one or more of Arg, Asn, Asp, His, Met, Leu, Val, Gln, Tyr, Thr, Lys, Ala, Ser, and Phe. The present invention includes a transgenic soybean plant produced by the method.

The present invention includes a method for preparing a transgenic dicot plant having an increased amino acid content, comprising the steps of: a) introducing into regenerable cells of a dicot plant a transgene comprising a construct comprising a polynucleotide encoding a monomeric AHAS, or a construct comprising a polynucleotide encoding a large subunit of AHAS and a polynucleotide encoding a small subunit of AHAS; b) regenerating said regenerable cell into a dicot plant; c) harvesting seed from said plant; d) selecting one or more seeds with an increased level of amino acid as compared to a seed from a non-transgenic plant of the same plant species; and e) planting said seed. In one embodiment, the dicot plant is a soybean plant or canola plant. In one embodiment, the increased level of amino acids comprises an increase in the concentration of Ser or Val. In one embodiment, the present invention includes a transgenic soybean plant produced by the method.

The present invention also includes meal produced from the transgenic soybeans.

The present invention is also directed to a container containing seeds of the present invention. Seeds of a plant or plants of the present invention may be placed in a container, such as, for example, a bag. As used herein, a container is any object capable of holding such seeds. A container preferably contains greater than about 1,000, about 5,000, or about 25,000 seeds where at least about 10%, about 25%, about 50%, about 75%, or about 100% of the seeds are seeds of the present invention. Preferably, where the seeds of the present invention are soybeans, the container is preferably a bag that contains about 60 pounds or about 130,000 beans.

The present invention is further directed to animal or human food products made from the transgenic plants or plant parts (e.g., seeds) of the present invention. Such food products can be made from, for example, grain, meal, flour, seed, cereal, and the like, including intermediate products made from such materials.

### BRIEF DESCRIPTION OF THE FIGURES

FIG. 1 is a restriction map of plasmid pMON53905.

FIG. 2 is a restriction map of plasmid pMON25666.

FIG. 3 is a restriction map of plasmid pMON53910.

FIG. 4 is a restriction map of plasmid pMON53911.

FIG. 5 is a restriction map of plasmid pMON53912.

FIG. 6 illustrates the kinetic properties of Arabidopsis threonine deaminase (diamond symbols) and E. coli threonine deaminase (circular symbols) by providing a plot of the initial velocity of wild type enzymes vs. threonine concentration.

FIG. 7 provides a plot of the percent enzymatic activity for E. coli L481 alleles vs. isoleucine concentration.

FIG. 8 is a restriction map of plasmid pMON69657.

FIG. 9 is a restriction map of plasmid pMON69659.

FIG. 10 is a restriction map of plasmid pMON69660.

FIG. 11 is a restriction map of plasmid pMON69663.

FIG. 12 is a restriction map of plasmid pMON69664.

FIG. 13 is a restriction map of plasmid pMON58143.

FIG. 14 is a restriction map of plasmid pMON58138.

FIG. 15 is a restriction map of plasmid pMON58159.

FIG. 16 is a restriction map of plasmid pMON58162.

### DESCRIPTION OF THE NUCLEIC ACID AND PEPTIDE SEQUENCES

SEQ ID NO: 1 represents a polynucleotide sequence for the wild type E. coli threonine deaminase.

SEQ ID NO: 2 represents an amino acid sequence for the wild type E. coli threonine deaminase.

SEQ ID NO: 3 represents an amino acid sequence for the wild type E. coli threonine deaminase having a Phe replacing the Leu at position 447, (Ilv219).

SEQ ID NO: 4 represents an amino acid sequence for the wild type E. coli threonine deaminase having a Phe replacing the Leu at position 481, (Ilv466).

SEQ ID NO: 5 represents an amino acid sequence for the wild type E. coli threonine deaminase having a Tyr replacing the Leu at position 481.

SEQ ID NO: 6 represents an amino acid sequence for the wild type E. coli threonine deaminase having a Pro replacing the Leu at position 481.

SEQ ID NO: 7 represents an amino acid sequence for the wild type E. coli threonine deaminase having a Glu replacing the Leu at position 481.

SEQ ID NO: 8 represents an amino acid sequence for the wild type E. coli threonine deaminase having a Thr replacing the Leu at position 481.

SEQ ID NO: 9 represents an amino acid sequence for the wild type E. coli threonine deaminase having a Gln replacing the Leu at position 481.

SEQ ID NO: 10 represents an amino acid sequence for the wild type E. coli threonine deaminase having an Ile replacing the Leu at position 481.

SEQ ID NO: 11 represents an amino acid sequence for the wild type E. coli threonine deaminase having a Val replacing the Leu at position 481.

SEQ ID NO: 12 represents an amino acid sequence for the wild type E. coli threonine deaminase having a Met replacing the Leu at position 481.

SEQ ID NO: 13 represents an amino acid sequence for the wild type E. coli threonine deaminase having a Lys replacing the Leu at position 481.

SEQ ID NO: 14 represents a polynucleotide sequence for the L447F E. coli threonine deaminase having a Phe replacing the Leu at position 447.

SEQ ID NO: 15 represents a polynucleotide sequence for the L481F E. coli threonine deaminase having a Phe replacing the Leu at position 481.

SEQ ID NO: 16 represents a polynucleotide sequence for an ilvG AHAS large subunit.

SEQ ID NO: 17 represents a polynucleotide sequence for an ilvM AHAS small subunit.

SEQ ID NO: 18 represents a polynucleotide sequence for an ilvG 5′ fragment.

SEQ ID NO: 19 represents a polynucleotide sequence for an Arabidopsis SSU1A plastid transit peptide.

SEQ ID NO: 20 represents a polynucleotide sequence for an ilvG 3′ fragment.

SEQ ID NO: 21 represents an amino acid sequence variant for the wild type E. coli threonine deaminase.

SEQ ID NO: 22 represents a polynucleotide sequence for the Arabidopsis OMR1 threonine deaminase.

### DETAILED DESCRIPTION OF THE INVENTION

The present invention provides a transgenic plant, the genome of which has an isolated nucleic acid encoding a threonine deaminase (TD), or subunit thereof, including enzymatically functional mutants and subunits. Such a threonine deaminase or threonine deaminase subunit is preferably resistant to inhibition by free L-isoleucine or an amino acid analog of isoleucine. An alternative preferred embodiment has the nucleic acid that encodes the threonine deaminase, or subunit thereof, expressed in a manner that the Ile content and the content of one or more of Arg, Asn, Asp, His, Met, Ala, Leu, Thr, Val, Gln, Tyr, Lys, Ser, and Phe of the plant increase irrespective of differences or similarities of kinetics or inhibition characteristics of the native and exogenous threonine deaminase, or subunit thereof. For example, using techniques well known in the art, the exogenous threonine deaminase enzyme could be caused to express predominantly in cellular compartments that are separate from the location of the native enzyme. Expression of the threonine deaminase, or subunit thereof, can elevate the level of Ile and elevate the level of one or more of Arg, Asn, Asp, His, Met, Ala, Leu, Thr, Val, Gln, Tyr, Lys, Ser, and Phe in the plant over the level present in the absence of such expression. The nucleic acid may also encode other enzymes involved in the biosynthesis of isoleucine, for example, aspartate kinase, bifunctional aspartate kinase—homoserine dehydrogenase, or acetohydroxy acid synthase.

The present invention also relates to a method for obtaining plants that produce elevated levels of free Ile and elevated level of one or more of Arg, Asn, Asp, His, Met, Ala, Leu, Thr, Val, Gln, Tyr, Lys, Ser, and Phe. Such overproduction results from the introduction and expression of an isolated nucleic acid encoding threonine deaminase. Moreover, native soybean threonine deaminase is sensitive to feedback inhibition by L-isoleucine and constitutes a site of regulation of the biosynthetic pathway. The methods provided in the present invention may also be used to produce increased levels of free Ile and increased levels of one or more of Arg, Asn, Asp, His, Met, Ala, Leu, Thr, Val, Gln, Tyr, Lys, Ser, and Phe in plants by introduction of a nucleic acid encoding a threonine deaminase that is resistant to such feedback inhibition. Such threonine deaminase encoding nucleic acids can be introduced into a variety of plants, including dicots (e.g., legumes) as well as monocots (e.g., cereal grains).

Definitions

In the context of this disclosure, a number of terms shall be utilized. The terms “polynucleotide”, “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural, or altered nucleotide bases. A polynucleotide in the form of a polymer of DNA may be comprised of one or more segments of cDNA, genomic DNA, synthetic DNA, or mixtures thereof.

As used herein, “altered” levels of Ile and one or more of Arg, Asn, Asp, His, Met, Ala, Leu, Thr, Val, Gln, Tyr, Lys, Ser, and Phe in a transformed plant, plant tissue, plant part, or plant cell are levels that are greater or lesser than the levels found in the corresponding untransformed plant, plant tissue, plant part, or plant cell. In general, “altered” levels of Ile and one or more of Arg, Asn, Asp, His, Met, Ala, Leu, Thr, Val, Gln, Tyr, Lys, Ser, and Phe are greater than the levels found in the corresponding untransformed plant, plant tissue, or plant cells.

The term “complementary to” is used herein to mean that the sequence of a nucleic acid strand could hybridize to all, or a portion, of a reference polynucleotide sequence. For illustration, the nucleotide sequence “TATAC” has 100% identity to a reference sequence 5′-TATAC-3′ but is 100% complementary to a reference sequence 5′-GTATA-3′.

The term “corresponds to” is used herein to mean that a polynucleotide, e.g., a nucleic acid, is at least partially identical (not necessarily strictly evolutionarily related) to all or a portion of a reference polynucleotide sequence.

As used herein, “deregulated enzyme” refers to an enzyme that has been modified, for example by mutagenesis, truncation and the like, so that the extent of feedback inhibition of the catalytic activity of the enzyme by a metabolite is reduced such that the enzyme exhibits enhanced activity in the presence of the metabolite as compared to the unmodified enzyme.

As used herein with respect to threonine deaminase, the phrase “a domain thereof” includes a structural or functional segment of a full-length threonine deaminase. A structural domain includes an identifiable structure within the threonine deaminase. An example of a structural domain includes an alpha helix, a beta sheet, an active site, a substrate or inhibitor binding site, and the like. A functional domain includes a segment of a threonine deaminase that performs an identifiable function such as an isoleucine binding pocket, an active site or a substrate, or inhibitor binding site. Functional domains of threonine deaminase include those portions of threonine deaminase that can catalyze one step in the biosynthetic pathway of isoleucine. Hence, a functional domain includes enzymatically active fragments and domains of threonine deaminase. Mutant domains of threonine deaminase are also contemplated. Wild type threonine deaminase nucleic acids utilized to make mutant domains include, for example, any nucleic acid encoding a domain of threonine deaminase from Escherichia coli, Salmonella typhimurium, or Arabidopsis thaliana.

As used herein, an “exogenous” threonine deaminase is a threonine deaminase that is encoded by an isolated nucleic acid that has been introduced into a host cell. Such an “exogenous” threonine deaminase is generally not identical to any DNA sequence present in the cell in its native, untransformed state. An “endogenous” or “native” threonine deaminase is a threonine deaminase that is naturally present in a host cell or organism.

As used herein, “increased” or “elevated” levels of free Ile and one or more of Arg, Asn, Asp, His, Met, Ala, Leu, Thr, Val, Gln, Tyr, Lys, Ser, and Phe in a plant cell, plant tissue, plant part, or plant are levels that are about 2 to 100 times, preferably about 5 to 50 times, and more preferably about 10-30 times, the levels found in an untransformed plant cell, plant tissue, plant part, or plant, i.e., one where the genome has not been altered by the presence of an exogenous threonine deaminase nucleic acid or domain thereof. For example, the levels of free Ile and one or more of Arg, Asn, Asp, His, Met, Ala, Leu, Thr, Val, Gln, Tyr, Lys, Ser, and Phe in a transformed plant seed are compared with those in an untransformed parent plant seed or with an untransformed seed in a chimeric plant. The names of the various amino acids found in plants and described in the present invention, their 3 and 1 letter abbreviations, as well as DNA codons that encode them are provided in Table 1.

TABLE 1

The names of the various amino acids found in
plants, their 3 and 1 letter abbreviations,
as well as the DNA codons that encode them.

| | 3 Letter | 1 Letter | |
|---|---|---|---|
| | Abbrevi- | Abbrevi- | |
| Amino Acid | ation | ation | DNA codons |
| Alanine | Ala | A | GCT, GCC, GCA, GCG |
| Arginine | Arg | R | CGT, CGC, CGA, CGG, |
| | | | AGA, AGG |
| Asparagine | Asn | N | AAT, AAC |
| Aspartic | Asp | D | GAT, GAG |
| acid | | | |
| Cysteine | Cys | C | TGT, TGC |
| Glutamic | Glu | E | GAA, GAG |
| acid | | | |
| Glutamine | Gln | Q | CAA, CAG |
| Glycine | Gly | G | GGT, GGC, GGA, GGG |
| Histidine | His | H | CAT, CAC |
| Isoleucine | Iso | I | ATT, ATC, ATA |
| Leucine | Leu | L | CTT, CTC, CTA, CTG, |
| | | | TTA, TTG |
| Lysine | Lys | K | AAA, AAG |
| Methionine | Met | M | ATG |
| Phenylala- | Phe | F | TTT, TTC |
| nine | | | |
| Proline | Pro | P | CCT, CCC, CCA, CCG |
| Serine | Ser | S | TCT, TCC, TCA, TCG, |
| | | | AGT, AGC |
| Threonine | Thr | T | ACT, ACC, ACA, ACG |
| Tryptophan | Trp | W | TGG |
| Tyrosine | Tyr | Y | TAT, TAC |
| Valine | Val | V | GTT, GTC, GTA, GTG |

Nucleic acids encoding a threonine deaminase, and nucleic acids encoding a transit peptide or marker/reporter gene are “isolated” in that they were taken from their natural source and are no longer within the cell where they normally exist. Such isolated nucleic acids may have been at least partially prepared or manipulated in vitro, e.g., isolated from a cell in which they are normally found, purified, and amplified. Such isolated nucleic acids can also be “recombinant” in that they have been combined with exogenous nucleic acids. For example, a recombinant DNA can be an isolated DNA that is operably linked to an exogenous promoter or to a promoter that is endogenous to a selected host cell.

As used herein, a “native” gene or nucleic acid means that the gene or nucleic acid has not been changed or manipulated in vitro, i.e., it is a “wild type” gene or nucleic acid that has not been isolated, purified, amplified, or mutated in vitro.

The term “plastid” refers to the class of plant cell organelles that includes amyloplasts, chloroplasts, chromoplasts, elaioplasts, eoplasts, etioplasts, leucoplasts, and proplastids. These organelles are self-replicating, and contain what is commonly referred to as a “plastid genome”, a circular DNA molecule that ranges in size from about 120 to about 217 kb, depending upon the plant species, and which usually contains an inverted repeat region.

As used herein, “polypeptide” means a continuous chain of amino acids that are all linked together by peptide bonds, except for the N-terminal and C-terminal amino acids that have amino and carboxylate groups, respectively, and that are not linked in peptide bonds. Polypeptides can have any length and can be post-translationally modified, for example, by glycosylation or phosphorylation.

As used herein, a plant cell, plant tissue, or plant that is “resistant or tolerant to inhibition by an amino acid analog of isoleucine” is a plant cell, plant tissue, or plant that retains at least about 10% more threonine deaminase activity in the presence of Lisoleucine or an analog of L-isoleucine, than a corresponding wild type threonine deaminase. In general, a plant cell, plant tissue, or plant that is “resistant or tolerant to inhibition by isoleucine” can grow in an amount of an amino acid analog of isoleucine that normally inhibits growth of the untransformed plant cell, plant tissue, or plant, as determined by methodologies known to the art. For example, a homozygous backcross converted inbred plant transformed with a DNA molecule that encodes a threonine deaminase that is substantially resistant or tolerant to inhibition by an amino acid analog of isoleucine grows in an amount of an amino acid analog of isoleucine that inhibits the growth of the corresponding, i.e., substantially isogenic, recurrent inbred plant.

As used herein, a threonine deaminase that is “resistant or tolerant to inhibition by isoleucine or an amino acid analog of isoleucine” is a threonine deaminase that retains greater than about 10% more activity than a corresponding “wild type” or native susceptible threonine deaminase, when the tolerant/resistant and wild type threonine deaminases are exposed to equivalent amounts of isoleucine or an amino acid analog of isoleucine. Preferably the resistant or tolerant threonine deaminase retains greater than about 20% more activity than a corresponding “wild type” or native susceptible threonine deaminase.

General Concepts

The preselected threonine deaminase nucleic acid must first be isolated and, if not of plant origin, be modified in vitro to include regulatory signals required for gene expression in plant cells. The exogenous gene may be modified to add sequences encoding a plastid transit peptide sequence in order to direct the gene product to these organelles.

In order to alter the biosynthesis of Ile and one or more of Arg, Asn, Asp, His, Met, Ala, Leu, Thr, Val, Gln, Tyr, Lys, Ser, and Phe, the nucleic acid encoding resistant threonine deaminase (“the gene”) must be introduced into the plant cells and these transformed cells identified, either directly or indirectly. The gene can be stably incorporated into the plant cell genome. The transcriptional signals of the gene must be recognized by and be functional in the plant cells. That is, the gene must be transcribed into messenger RNA, and the mRNA must be stable in the plant nucleus and be transported intact to the cytoplasm for translation. The gene can have appropriate translational signals to be recognized and properly translated by plant cell ribosomes. The polypeptide gene product must escape significant proteolytic attack in the cytoplasm and be able to assume a three-dimensional conformation that will confer enzymatic activity. The threonine deaminase further can function in the biosynthesis of isoleucine and its derivatives; that is, it can be localized near the native plant enzymes catalyzing the flanking steps in biosynthesis (presumably in the plastid) in order to obtain the required substrates and to pass on the appropriate product.

Even if all these conditions are met, successful overproduction of Ile and one or more of Arg, Asn, Asp, His, Met, Ala, Leu, Thr, Val, Gln, Tyr, Lys, Ser, and Phe is not a predictable event. There must be no other control mechanism compensating for the reduced regulation at the threonine deaminase step. This means not only no other inhibition of biosynthesis, but also no mechanism to increase the rate of breakdown of the accumulated amino acids. Ile and one or more of Arg, Asn, Asp, His, Met, Ala, Leu, Thr, Val, Gln, Tyr, Lys, Ser, and Phe must be also overproduced at levels that are not toxic to the plant. Finally, the introduced trait must be stable and heritable in order to permit commercial development and use.

Isolation and Identification of Polynucleic Acid Molecules Encoding a Threonine Deaminase

Nucleic acids encoding a threonine deaminase can be identified and isolated by standard methods, as described by Sambrook et al., Molecular Cloning: A Laboratory Manual, Cold Spring Harbor, N.Y. (2001). Nucleic acids encoding a threonine deaminase can be from any prokaryotic or eukaryotic species. For example, a nucleic acid encoding a threonine deaminase, or subunit thereof, can be identified by screening of a genomic DNA library derived from any species or by screening a cDNA library generated from nucleic acid derived from a particular cell type, cell line, primary cells, or tissue. Examples of libraries useful for identifying and isolating a threonine deaminase include, but are not limited to, a cDNA library derived from A. tumefaciens strain A348, maize inbred line B73 (Stratagene, La Jolla, Calif., Cat. #937005, Clontech, Palo Alto, Calif., Cat. # FL1032a, #FL1032b, and FL1032n), a genomic library from maize inbred line Mo17 (Stratagene, Cat. #946102), a genomic library from maize inbred line B73 (Clontech, Cat. # FL1032d), or a genomic library from a convenient strain of Escherichia coli or Salmonella typhimurium.

Examples of threonine deaminase polynucleotide or polypeptide molecules useful for practice of the present invention are described in Table 2. The E. coli wild type threonine deaminase gene (ilvA) (SEQ ID NO: 1; gi:146450, accession K03503, version K03503.1) and its corresponding polypeptide sequence (SEQ ID NO: 2) or a variant allele encoding SEQ ID NO: 21, is the base gene from which all other mutant alleles described in Table 2 below were derived.

Nucleic acids having sequences related to these threonine deaminase nucleic acid molecules can be obtained by standard methods, including cloning or polymerase chain reaction (PCR) using oligonucleotide primers complementary to regions of threonine deaminase sequences provided herein. The sequence of an isolated threonine deaminase nucleic acid can be verified by hybridization, partial sequence analysis, or by expression in an appropriate host cell.

TABLE 2
E. coli ilvA threonine deaminase amino acid substitutions in mutant alleles

| Threonine | | |
|---|---|---|
| Deaminase | | SEQ |
| Mutation | Description of Mutant Allele | ID NO: |
| E. coli (wt ilvA) | Wild type E. coli TD nucleic acid sequence | 1 |
| E. coli (wt ilvA) | Wild type E. coli TD polypeptide sequence | 2 |
| L447F (ilvA219) | Leu at position 447 replaced with Phe | 3 |
| L481F (ilvA466) | Leu at position 481 replaced with Phe | 4 |
| L481Y | Leu at position 481 replaced with Tyr | 5 |
| L481P | Leu at position 481 replaced with Pro | 6 |
| L481E | Leu at position 481 replaced with Glu | 7 |
| L481T | Leu at position 481 replaced with Thr | 8 |
| L481Q | Leu at position 481 replaced with Gln | 9 |
| L481I | Leu at position 481 replaced with Ile | 10 |
| L481V | Leu at position 481 replaced with Val | 11 |
| L481M | Leu at position 481 replaced with Met | 12 |

Screening for DNA fragments that encode all or a portion of the sequence encoding a threonine deaminase can be accomplished by PCR, or by screening plaques from a genomic or cDNA library using hybridization procedures. The probe can be derived from a threonine deaminase gene obtained from the nucleic acids provided herein or from other organisms. Alternatively, plaques from a cDNA expression library can be screened for binding to antibodies that specifically bind to threonine deaminase. DNA fragments that hybridize to threonine deaminase probes from other organisms, and/or plaques carrying DNA fragments that are immunoreactive with antibodies to threonine deaminase, can be subcloned into a vector and sequenced and/or used as probes to identify other cDNA or genomic sequences encoding all or a portion of the desired threonine deaminase gene.

A cDNA library can be prepared by isolation of mRNA, generation of cDNA, and insertion of cDNA into an appropriate vector. The library containing cDNA fragments can be screened with probes or antibodies specific for threonine deaminase. DNA fragments encoding a portion of a threonine deaminase gene can be subcloned and sequenced and used as probes to identify a genomic threonine deaminase nucleic acid. DNA fragments encoding a portion of a prokaryotic or eukaryotic threonine deaminase can be verified by determining sequence homology with other known threonine deaminase genes or by hybridization to threonine deaminase-specific messenger RNA. Once cDNA fragments encoding portions of the 5′, middle and 3′ ends of a threonine deaminase are obtained, they can be used as probes to identify and clone a complete genomic copy of the threonine deaminase gene from a genomic library.

Portions of the genomic copy or copies of a threonine deaminase gene can be isolated by polymerase chain reaction or by screening a genomic library. Positive clones can be sequenced and the 5′ end of the gene identified by standard methods including either nucleic acid homology to other threonine deaminase genes or by RNAase protection analysis, as described by Sambrook et al., Molecular Cloning: A Laboratory Manual, Cold Spring Harbor, N.Y. (1989 and 2001). The 3′ and 5′ ends of the target gene can also be located by computer searches of genomic sequence databases using known threonine deaminase coding regions. Once portions of the gene are identified, complete copies of the threonine deaminase gene can be obtained by standard methods, including cloning or polymerase chain reaction (PCR) synthesis using oligonucleotide primers complementary to the nucleic acid at the 5′ or 3′ end of the gene. The presence of an isolated full-length copy of the threonine deaminase gene can be verified by hybridization, partial sequence analysis, or by expression of the threonine deaminase enzyme.

Mutants having increased threonine deaminase activity, reduced sensitivity to feedback inhibition by isoleucine or analogs thereof, and/or the ability to generate increased amounts of Ile and one or more of Arg, Asn, Asp, His, Met, Ala, Leu, Thr, Val, Gln, Tyr, Lys, Ser, and Phe in a plant are desirable. Such mutants can have a functional change in the level or type of activity they exhibit and are sometimes referred to as “derivatives” of wild type threonine deaminase nucleic acids and polypeptides.

However, the present invention also contemplates threonine deaminase variants as well as threonine deaminase nucleic acids with “silent” mutations. As used herein, a silent mutation is a mutation that changes the nucleotide sequence of the threonine deaminase but that does not change the amino acid sequence of the encoded threonine deaminase. A variant threonine deaminase is encoded by a mutant nucleic acid and the variant has one or more amino acid changes that do not substantially change the threonine deaminase activity when compared to the corresponding wild type threonine deaminase. The present invention is directed to all such derivatives, variants, and threonine deaminases nucleic acids with silent mutations.

DNA encoding a mutated threonine deaminase that is resistant and/or tolerant to L-isoleucine or amino acid analogs of isoleucine can be obtained by several methods. The methods include, but are not limited to:

1. spontaneous variation and direct mutant selection in cultures;

2. direct or indirect mutagenesis procedures on tissue cultures of any cell types or tissue, seeds, or plants;

3. mutation of the cloned threonine deaminase gene by methods such as by chemical mutagenesis; site specific or site directed mutagenesis Sambrook et al., cited supra), transposon mediated mutagenesis (Berg et al., Biotechnology, 1:417 (1983)), and deletion mutagenesis (Mitra et al., Molec. Gen. Genetic., 215:294 (1989));

4. rational design of mutations in key residues; and

5. DNA shuffling to incorporate mutations of interest into various threonine deaminase nucleic acids.

For example, genetic and/or protein structural information from available threonine deaminase proteins can be used to rationally design threonine deaminase mutants that have a high probability of having increased activity or reduced sensitivity to isoleucine or isoleucine analogs. Such protein structural information is available, for example, on the E. coli threonine deaminase (Gallagher et al., Structure, 6:465-475 (1998)). Rational design of mutations can be accomplished by alignment of the selected threonine deaminase amino acid sequence with the threonine deaminase amino acid sequence from a threonine deaminase of known structure, for example, E. coli. The predicted isoleucine binding and catalysis regions of the threonine deaminase protein can be assigned by combining the knowledge of the structural information with the sequence homology. For example, residues in the isoleucine-binding pocket can be identified as potential candidates for mutation to alter the resistance of the enzyme to feedback inhibition by isoleucine. Using such structural information, several E. coli threonine deaminase mutants were rationally designed in the site or domain involved in isoleucine binding. More specifically, amino acids analogous to L481 in the E. coli threonine deaminase are being potentially useful residues for mutation to produce active threonine deaminases that may have less sensitivity to isoleucine feedback inhibition. The present invention contemplates any amino acid substitution or insertion at any of these positions. Alternatively, the amino acid at any of these positions can be deleted as well as substituted.

Site directed mutagenesis can be used to generate amino acid substitutions, deletions, and insertions at a variety of sites. Examples of specific mutations made within the Escherichia coli threonine deaminase coding region include the following:
at about position 447 replace Leu with Phe (see, e.g., SEQ ID NO: 3);
at about position 481 replace Leu with Phe (see, e.g., SEQ ID NO: 4);
at about position 481 replace Leu with Tyr (see, e.g., SEQ ID NO: 5);
at about position 481 replace Leu with Pro (see, e.g., SEQ ID NO: 6);
at about position 481 replace Leu with Glu (see, e.g., SEQ ID NO: 7);
at about position 481 replace Leu with Thr (see, e.g., SEQ ID NO: 8);
at about position 481 replace Leu with Gln (see, e.g., SEQ ID NO: 9);
at about position 481 replace Leu with Ile (see, e.g., SEQ ID NO: 10);
at about position 481 replace Leu with Val (see, e.g., SEQ ID NO: 11);
at about position 481 replace Leu with Met (see, e.g., SEQ ID NO: 12); or
at about position 481 replace Leu with Lys (see, e.g., SEQ ID NO: 13).

Similar mutations can be made in analogous positions of any threonine deaminase by alignment of the amino acid sequence of the threonine deaminase to be mutated with an E. coli threonine deaminase amino acid sequence. One example of an E. coli threonine deaminase amino acid sequence that can be used for alignment is SEQ ID NO: 1.

Useful mutants can also be identified by classical mutagenesis and genetic selection. A functional change can be detected in the activity of the enzyme encoded by the gene by exposing the enzyme to free L-isoleucine or amino acid analogs of isoleucine, or by detecting a change in the DNA molecule using restriction enzyme mapping or DNA sequence analysis.

For example, a gene encoding a threonine deaminase substantially tolerant to isoleucine can be isolated from a cell line that is tolerant to an isoleucine analog. Briefly, partially differentiated plant cell cultures are grown and subcultured with continuous exposure to low levels of the isoleucine analog. The concentration of the isoleucine analog is then gradually increased over several subculture intervals. Cells or tissues growing in the presence of normally toxic levels of the analog are repeatedly subcultured in the presence of the analog and characterized. Stability of the tolerance trait of the cultured cells may be evaluated by growing the selected cell lines in the absence of the analog for varying periods of time and then analyzing growth after exposing the tissue to the analog. Cell lines that are tolerant by virtue of having an altered threonine deaminase enzyme can be selected by identifying cell lines having enzyme activity in the presence of normally toxic, i.e., growth inhibitor, levels of the isoleucine analog.

The threonine deaminase gene cloned from an isoleucine analog resistant cell line can be assessed for tolerance to the same or other amino acid analog(s) by standard methods, as described in U.S. Pat. No. 4,581,847, the disclosure of which is incorporated by reference herein.

Cell lines with a threonine deaminase having reduced sensitivity to analogs of isoleucine can be used to isolate a feedback-resistant threonine deaminase. A DNA library from a cell line tolerant to an isoleucine analog can be generated and DNA fragments encoding all or a portion of a threonine deaminase gene can be identified by hybridization to a cDNA probe encoding a portion of a threonine deaminase gene. A complete copy of the altered gene can be obtained by cloning procedures or by PCR synthesis using appropriate primers. The isolation of the altered gene coding for threonine deaminase can be confirmed in transformed plant cells by determining whether the threonine deaminase being expressed retains enzyme activity when exposed to normally toxic levels of the isoleucine analog. See, for example, Anderson et al., U.S. Pat. No. 6,118,047.

Coding regions of any DNA molecule provided herein can also be optimized for expression in a selected organism, for example, a selected plant or other host cell type.

The generation of variants of threonine deaminase that are isoleucine-deregulated is also described in U.S. Pat. Nos. 5,942,660 and 5,958,745 by Gruys et al., by Asrar et al., U.S. Pat. Nos. 6,091,002 and 6,228,623; and by Slater et al., Nature Biotechnology, 17:1011 (1999).

Transgenes and Vectors

Once a nucleic acid encoding, e.g., threonine deaminase or a domain thereof, is obtained and amplified, it is operably linked to a promoter and, optionally, linked with other elements to form a transgene.

Most genes have regions that are known as promoters and which regulate gene expression. Promoter regions are typically found upstream from the coding sequence in both prokaryotic and eukaryotic cells. A promoter sequence provides for regulation of transcription of the downstream gene sequence and typically includes from about 50 to about 2,000 nucleotide base pairs. Promoter sequences also contain regulatory sequences such as enhancer sequences that can influence the level of gene expression. Some isolated promoter sequences can provide for gene expression of heterologous genes, that is, a gene different from the native or homologous gene. Promoter sequences are also known to be strong or weak or inducible. A strong promoter provides for a high level of gene expression, whereas a weak promoter provides for a very low level of gene expression. An inducible promoter is a promoter that permits turning gene expression on and off in response to an exogenously added agent or to an environmental or developmental stimulus. Promoters can also provide for tissue specific or developmental regulation. A strong promoter that provides for a sufficient level of gene expression and easy detection and selection of transformed cells may be advantageous. Also, such a strong promoter may provide high levels of gene expression when desired.

The promoter in a transgene of the present invention can provide for expression of a gene of interest, e.g., threonine deaminase from a nucleic acid encoding threonine deaminase. Preferably, the coding sequence is expressed so as to result in an increase in tolerance of the plant cells to feedback inhibition by free L-isoleucine so as to result in an increase in the total Ile and one or more of Arg, Asn, Asp, His, Met, Ala, Leu, Thr, Val, Gln, Tyr, Lys, Ser, and Phe content of the cells. The promoter can also be inducible so that gene expression can be turned on or off by an exogenously added agent. It may also be desirable to combine the coding region with a promoter that provides tissue specific expression or developmentally regulated gene expression in plants.

Promoters useful in the present invention include, but are not limited to, viral, plastid, bacterial, bacteriophage, or plant promoters. Useful promoters include the CaMV 35S promoter (Odell et al., Nature, 313:810 (1985)), the CaMV 19S (Lawton et al., Plant Mol. Biol., 9:31F (1987)), nos (Ebert et al., Proc. Nat. Acad. Sci. (U.S.A.), 84:5745 (1987)), Adh (Walker et al., Proc. Nat. Acad. Sci. (U.S.A.), 84:6624 (1987)), sucrose synthase (Yang et al., Proc. Nat. Acad. Sci. (U.S.A.), 87:4144 (1990)), α-tubulin, napin, actin (Wang et al., Mol. Cell. Biol., 12:3399 (1992)), cab (Sullivan et al., Mol. Gen. Genet., 215:431 (1989)), PEPCase promoter (Hudspeth et al., Plant Mol. Biol., 12:579 (1989)), the 7Sα′ conglycinin promoter (Beachy et al., EMBO J., 4:3047 (1985)), or those associated with the R gene complex (Chandler et al., The Plant Cell, 1:1175 (1989)). Preferred promoters include seed enhanced promoters, for example, soybean 7sα′, 7sα, lea9, Arabidopsis per1, and Brassica napus napin. It is contemplated that other promoters useful in the practice of the present invention are available to those of skill in the art.

Plastid promoters can also be used. Most plastid genes contain a promoter for the multi-subunit plastid-encoded RNA polymerase (PEP) as well as the single-subunit nuclear-encoded RNA polymerase. A consensus sequence for the nuclear-encoded polymerase (NEP) promoters and listing of specific promoter sequences for several native plastid genes can be found in Hajdukiewicz et al., EMBO J., 16:4041-4048 (1997), which is hereby in its entirety incorporated by reference.

Examples of plastid promoters that can be used include the Zea mays plastid RRN (ZMRRN) promoter. The ZMRRN promoter can drive expression of a gene when the Arabidopsis thaliana plastid RNA polymerase is present. Similar promoters that can be used in the present invention are the Glycine max plastid RRN (SOYRRN) and the Nicotiana tabacum plastid RRN (NTRRN) promoters. All three promoters can be recognized by the Arabidopsis plastid RNA polymerase. The general features of RRN promoters are described in U.S. Pat. No. 6,218,145.

Moreover, transcription enhancers or duplications of enhancers can be used to increase expression from a particular promoter. Examples of such enhancers include, but are not limited to, elements from the CaMV 35S promoter and octopine synthase genes (Last et al., U.S. Pat. No. 5,290,924). For example, it is contemplated that vectors for use in accordance with the present invention may be constructed to include the ocs enhancer element. This element was first identified as a 16 bp palindromic enhancer from the octopine synthase (ocs) gene of Agrobacterium (Ellis et al., EMBO J., 6:3203 (1987)), and is present in at least 10 other promoters (Bouchez et al., EMBO J., 8:4197 (1989)). It is proposed that the use of an enhancer element, such as the ocs element and particularly multiple copies of the element, will act to increase the level of transcription from adjacent promoters when applied in the context of monocot transformation. Tissue-specific promoters, including but not limited to, root-cell promoters (Conkling et al., Plant Physiol., 93:1203 (1990)), and tissue-specific enhancers (Fromm et al., The Plant Cell, 1:977 (1989)) are also contemplated to be particularly useful, as are inducible promoters such as ABA- and turgor-inducible promoters, and the like.

As the DNA sequence between the transcription initiation site and the start of the coding sequence, i.e., the untranslated leader sequence, can influence gene expression, one may also wish to employ a particular leader sequence. Preferred leader sequences are contemplated to include those which include sequences predicted to direct optimum expression of the attached gene, i.e., to include a preferred consensus leader sequence which may increase or maintain mRNA stability and prevent inappropriate initiation of translation (Joshi, Nucl. Acid Res., 15:6643 (1987)). The choice of such sequences can readily be made by those of skill in the art. Sequences that are derived from genes that are highly expressed in dicots and in soybean in particular, are preferred.

Nucleic acids encoding the gene of interest, e.g., threonine deaminase, can also include a plastid transit peptide to facilitate transport of the threonine deaminase polypeptide into plastids, for example, into chloroplasts. A nucleic acid encoding the selected plastid transit peptide is generally linked in-frame with the coding sequence of the threonine deaminase. However, the plastid transit peptide can be placed at either the N-terminal or C-terminal end of the threonine deaminase.

Constructs will also include the nucleic acid of interest along with a nucleic acid at the 3′ end that acts as a signal to terminate transcription and allow for the polyadenylation of the resultant mRNA. Examples of 3′ elements include those from the nopaline synthase gene of Agrobacterium tumefaciens (Bevan et al., Nucl. Acid Res., 11:369 (1983)), the terminator for the T7 transcript from the octopine synthase gene of Agrobacterium tumefaciens, and the 3′ end of the protease inhibitor I or inhibitor II genes from potato or tomato, although other 3′ elements known to those of skill in the art are also contemplated. Regulatory elements such as Adh intron 1 (Callis et al., Genes Develop., 1:1183 (1987)), sucrose synthase intron (Vasil et al., Plant Physiol., 91:5175 (1989)), or TMV omega element (Gallie et al., The Plant Cell, 1:301 (1989)) may further be included where desired. These 3′ nontranslated regulatory sequences can be obtained as described in An, Methods in Enzymology, 153:292 (1987) or are already present in plasmids available from commercial sources such as Clontech, Palo Alto, Calif. The 3′ nontranslated regulatory sequences can be operably linked to the 3′ terminus of a threonine deaminase gene by standard methods. Other such regulatory elements useful in the practice of the present invention are available to and may be used by those of skill in the art.

Selectable marker genes or reporter genes are also useful in the present invention. Such genes can impart a distinct phenotype to cells expressing the marker gene and thus allow such transformed cells to be distinguished from cells that do not have the marker. Selectable marker genes confer a trait that one can ‘select’ for by chemical means, i.e., through the use of a selective agent (e.g., a herbicide, antibiotic, or the like). Reporter genes or screenable genes, confer a trait that one can identify through observation or testing, i.e., by ‘screening’ (e.g., the R-locus trait). Of course, many examples of suitable marker genes are known to the art and can be employed in the practice of the present invention.

Possible selectable markers for use in connection with the present invention include, but are not limited to, a neo gene (Potrykus et al., Mol. Gen. Genet., 199:183 (1985)) which codes for neomycin resistance and can be selected for using neomycin, kanamycin, G418, and the like; a bar gene which codes for bialaphos resistance; a gene which encodes an altered EPSP synthase protein (Hinchee et al., Biotech., 6:915 (1988)) thus conferring glyphosate resistance; a nitrilase gene such as bxn from Klebsiella ozaenae that confers resistance to bromoxynil (Stalker et al., Science, 242:419 (1988)); a mutant acetolactate synthase gene (ALS) that confers resistance to imidazolinone, sulfonylurea, or other ALS-inhibiting chemicals (EP 0 154 204); a methotrexate-resistant DHFR gene (Thillet et al., J. Biol. Chem., 263:12500 (1988)); a dalapon dehalogenase gene that confers resistance to the herbicide dalapon; or a mutated threonine deaminase gene that confers resistance to 5-methyl isoleucine. Where a mutant EPSP synthase gene is employed, a suitable plastid or chloroplast transit peptide (CTP) should be fused to the EPSPS coding region.

In one embodiment, the selectable marker is resistance to N-phosphonomethyl-glycine, commonly referred to as glyphosate. Glyphosate inhibits the shikimic acid pathway that leads to the biosynthesis of aromatic compounds including amino acids and vitamins. Specifically, glyphosate inhibits the conversion of phosphoenolpyruvic acid and 3-phosphoshikimic acid to 5-enolpyruvyl-3-phosphoshikimic acid by inhibiting the enzyme 5-enolpyruvyl-3-phosphoshikimic acid synthase (EPSP synthase or EPSPS). It has been shown that glyphosate tolerant plants can be produced by inserting into the genome of the plant the capacity to produce a higher level of EPSP synthase which enzyme is preferably glyphosate tolerant (Shah et al., Science, 233:478-481 (1986)). Variants of the wild type EPSPS enzyme have been isolated which are glyphosate-tolerant as a result of alterations in the EPSPS amino acid coding sequence. See, Kishore et al., Ann. Rev. Biochem., 57:627-663 (1988); Schulz et al., Arch. Microbiol., 137:121-123 (1984); Sost et al., FEBS Lett., 173:238-241 (1984); Kishore et al., Fed. Proc., 45:1506 (1986).

The introduction into plants of a nucleic acid encoding a glyphosate tolerant EPSP synthase or a glyphosate degradation enzyme can make the plant tolerant to glyphosate. Methods for making glyphosate tolerant plants are available, for example, in U.S. Pat. Nos. 5,776,760 and 5,627,061; and WO 92/00377, the disclosures of which are hereby incorporated by reference.

Another illustrative embodiment of a selectable marker gene capable of being used in systems to select transformants is the genes that encode the enzyme phosphinothricin acetyltransferase, such as the bar gene from Streptomyces hygroscopicus or the pat gene from Streptomyces viridochromogenes (U.S. Pat. No. 5,550,318). The enzyme phosphinothricin acetyl transferase (PAT) inactivates the active ingredient in the herbicide bialaphos, phosphinothricin (PPT). PPT inhibits glutamine synthetase, (Murakami et al., Mol. Gen. Genet., 205:42 (1986); Twell et al., Plant Physiol., 91:1270 (1989)) causing rapid accumulation of ammonia and cell death.

Screenable markers that may be employed include, but are not limited to, a β-glucuronidase or uidA gene (GUS) which encodes an enzyme for which various chromogenic substrates are known; an R-locus gene, which encodes a product that regulates the production of anthocyanin pigments (red color) in plant tissues (Dellaporta et al., in Chromosome Structure and Function, pp. 263-282 (1988)); a β-lactamase gene (Sutcliffe, Proc. Nat. Acad. Sci. (U.S.A.), 75:3737 (1978)), which encodes an enzyme for which various chromogenic substrates are known (e.g., PADAC, a chromogenic cephalosporin); a xylE gene (Zukowsky et al., Proc. Nat. Acad. Sci. (U.S.A.), 80:1101 (1983)) which encodes a catechol dioxygenase that can convert chromogenic catechols; an α-amylase gene (Ikuta et al., Biotech., 8:241 (1990)); a tyrosinase gene (Katz et al., J. Gen. Microbiol., 129:2703 (1983)) which encodes an enzyme capable of oxidizing tyrosine to DOPA and dopaquinone which in turn condenses to form the easily detectable compound melanin; a β-galactosidase gene, which encodes an enzyme for which there are chromogenic substrates; a luciferase (lux) gene (Ow et al., Science, 234:856 (1986)), which allows for bioluminescence detection; or even an aequorin gene (Prasher et al., Biochem. Biophys. Res. Comm., 126:1259 (1985)), which may be employed in calcium-sensitive bioluminescence detection, or a green fluorescent protein gene (Niedz et al., Plant Cell Reports, 14:403 (1995)). The presence of the lux gene in transformed cells may be detected using, for example, X-ray film, scintillation counting, fluorescent spectrophotometry, low-light video cameras, photon-counting cameras, or multiwell luminometry. It is also envisioned that this system may be developed for population screening for bioluminescence, such as on tissue culture plates, or even for whole plant screening.

Additionally, transgenes may be constructed and employed to provide targeting of the gene product to an intracellular compartment within plant cells or to direct a protein to the extracellular environment. This will generally be achieved by joining a nucleic acid encoding a transit or signal peptide sequence to the coding sequence of a particular gene. The resultant transit, or signal, peptide will transport the protein to a particular intracellular, or extracellular destination, respectively. In many cases the transit, or signal, peptide is removed after facilitating transport of the protein into a cellular compartment. Transit or signal peptides act by facilitating the transport of proteins through intracellular membranes, e.g., vacuole, vesicle, plastid, and mitochondrial membranes, whereas signal peptides direct proteins through the extracellular membrane. By facilitating transport of the protein into compartments inside or outside the cell, these sequences may increase the accumulation of gene product.

A particular example of such a use concerns the direction of the gene of interest, e.g., a threonine deaminase to a particular organelle, such as the plastid rather than to the cytoplasm. This is exemplified by the use of the Arabidopsis SSU1A transit peptide, which confers plastid-specific targeting of proteins. Alternatively, the transgene can comprise a plastid transit peptide-encoding nucleic acid or a nucleic acid encoding the rbcS (RuBISCO) transit peptide operably linked between a promoter and the nucleic acid encoding a threonine deaminase (for a review of plastid targeting peptides, see, Heijne et al., Eur. J. Biochem., 180:535 (1989); Keegstra et al., Ann. Rev. Plant Physiol. Plant Mol. Biol., 40:471 (1989)). If the transgene is to be introduced into a plant cell, the transgene can also contain plant transcriptional termination and polyadenylation signals and translational signals linked to the 3′ terminus of a plant threonine deaminase gene.

An exogenous plastid transit peptide can be used which is not encoded within a native plant threonine deaminase gene. A plastid transit peptide is typically 40 to 70 amino acids in length and functions post-translationally to direct a protein to the plastid. The transit peptide is cleaved either during or just after import into the plastid to yield the mature protein. The complete copy of a gene encoding a plant threonine deaminase may contain a plastid transit peptide sequence. In that case, it may not be necessary to combine an exogenously obtained plastid transit peptide sequence into the transgene.

Exogenous plastid transit peptide encoding sequences can be obtained from a variety of plant nuclear genes, so long as the products of the genes are expressed as pre-proteins comprising an amino terminal transit peptide and are transported into a selected plastid. Examples of plant gene products known to include such transit peptide sequences include, but are not limited to, the small subunit of ribulose biphosphate carboxylase, ferredoxin, chlorophyll a/b binding protein, chloroplast ribosomal proteins encoded by nuclear genes, certain heat shock proteins, amino acid biosynthetic enzymes such as acetolactate acid synthase, 3-enolpyruvylphosphoshikimate synthase, dihydrodipicolinate synthase, and the like. Alternatively, the DNA fragment coding for the transit peptide may be chemically synthesized either wholly or in part from the known sequences of transit peptides such as those listed above.

Regardless of the source of the DNA fragment coding for the transit peptide, it should include a translation initiation codon and be expressed as an amino acid sequence that is recognized by and will function properly in plastids of the host plant. Attention should also be given to the amino acid sequence at the junction between the transit peptide and the threonine deaminase enzyme, where it is cleaved to yield the mature enzyme. Certain conserved amino acid sequences have been identified and may serve as a guideline. Precise fusion of the transit peptide coding sequence with the threonine deaminase coding region may require manipulation of one or both nucleic acids to introduce, for example, a convenient restriction site. This may be accomplished by methods including site-directed mutagenesis, insertion of chemically synthesized oligonucleotide linkers, and the like.

Once obtained, the plastid transit peptide sequence can be appropriately linked to the promoter and a threonine deaminase-coding region in a transgene using standard methods. A plasmid containing a promoter functional in plant cells and having multiple cloning sites downstream can be constructed or obtained from commercial sources. The plastid transit peptide sequence can be inserted downstream from the promoter using restriction enzymes. A threonine deaminase-coding region can then be inserted immediately downstream from and in frame with the 3′ terminus of the plastid transit peptide sequence, so that the plastid transit peptide is translationally fused to the amino terminus of the threonine deaminase. Once formed, the transgene can be subcloned into other plasmids or vectors.

It is contemplated that targeting of the gene product to an intracellular compartment within plant cells may also be achieved by direct delivery of a gene to the intracellular compartment. For example, plastid transformation of plants has been described by P. Maliga (Current Opinion in Plant Biology, 5:164-172 (2002)); Heifetz (Biochimie, 82:655-666 (2000)); Bock (J. Mol. Biol., 312:425-438 (2001)); and Daniell et al., (Trends in Plant Science, 7:84-91 (2002)).

After constructing a transgene containing a threonine deaminase gene and/or other gene of interest, the cassette can then be introduced into a plant cell. Depending on the type of plant cell, the level of gene expression, and the activity of the enzyme encoded by the gene, introduction of DNA encoding a threonine deaminase into the plant cell can confer tolerance to isoleucine or an amino acid analog of isoleucine, and alter the isoleucine content of the plant cell.

Several constructs contemplated in the present invention are described in Table 3.

TABLE 3
Constructs contemplated in the present invention.

| Species | Promoter | Coding Sequence | Terminator |
|---|---|---|---|
| Soybean | Lea9 | ilvA466 | NOS |
| Soybean | Per1 | ilvA466 | NOS |
| Soybean | Lea 9 | ilvA219 | NOS |
| Soybean | Per1 | ilvA219 | NOS |
| A. Thaliana | 7s | ilvAL481Q | NOS |
| A. Thaliana | 7s | ilvAL481F | NOS |
| A. Thaliana | 7s | ilvAL481P | NOS |
| A. Thaliana | 7s | ilvAL481Y | NOS |
| Species | pMON | Description | Construction |
| Soybean | 53910 | 7Sα′-ilvAwt-NOS | |
| Soybean | 53911 | 7Sα′-ilvA219-NOS | |
| Soybean | 53912 | 7Sα′-ilvA466-NOS | |
| Soybean | 58028 | napin-ilvA219-NOS | |
| Soybean | 58029 | napin-ilvA219-NOS, convergent | |
| A. thaliana | 58031 | napin-ilvA219-NOS | |
| Soybean/ | 58117 | napin-OMR-1 (TD-FBR)-NOS | |
| A. thaliana | | | |

TD—threonine deaminase
AHAS—acetohydroxy acid synthase
AK—aspartate kinase
HSDH—homoserine dehydrogenase
FBR—feedback resistant
Arc—Arcelin
Per1—peroxiredoxin
Lea—late embryogenesis abundant

Use of Combinations of Nucleic Acids

One embodiment of the present invention involves the combination of a nucleic acid encoding a threonine deaminase with the ilvG and/or ilvM genes of E. coli, which encode AHAS II (acetohydroxy acid synthase). Such acetohydroxy acid synthase enzymes are not subject to amino acid feedback inhibition and have a preference for 2-ketobutyrate as a substrate. In one embodiment, the activity is confined to a single fusion polypeptide. Another embodiment involves the combination of an amino acid insensitive aspartate kinase—homoserine dehydrogenase (AK-HSDH) with threonine deaminase and potentially with AHASII. In one embodiment, the mutant thrA1 gene from S. marcescens, (Omori and Komatubara, J. Bact., 175:959 (1993)) is the AK-HSDH allele. These nucleic acids may be translationally fused to plastid transit peptides.

The AHAS enzyme is known to be present throughout higher plants, as well as being found in a variety of microorganisms, such as the yeast Saccharomyces cerevisiae, and the enteric bacteria, E. coli and Salmonella typhimurium (U.S. Pat. No. 5,731,180). The genetic basis for the production of normal AHAS in a number of these species has also been well characterized. For example, in both E. coli and Salmonella typhimurium three isozymes of AHAS exist; two of these are sensitive to herbicides while a third is not. Each of these isozymes possesses one large and one small protein subunit; and map to the I1vIH, I1vGM and I1vBN operons. In yeast, the single AHAS isozyme has been mapped to the ILV2 locus. In each case, sensitive and resistant forms have been identified and sequences of the various alleles have been determined (Friden et al., Nucl. Acid Res., 13:3979-3998 (1985); Lawther et al., PNAS USA, 78:922-928 (1982); Squires et al., Nucl. Acids Res., 811:5299-5313 (1983); Wek et al., Nucl. Acids Res., 13:4011-4027 (1985); Falco and Dumas, Genetics, 109:21-35 (1985); Falco et al., Nucl. Acids Res., 13:4011-4027 (1985)).

In tobacco, AHAS function is encoded by two unlinked genes, SuRA and SuRB. There is substantial identity between the two genes, both at the nucleotide level and amino acid level in the mature protein, although the N-terminal, putative transit region differs more substantially (Lee et al., EMBO J., 7:1241-1248 (1988)). Arabidopsis, on the other hand, has a single AHAS gene, which has also been completely sequenced (Mazur et al., Plant Physiol., 85:1110-1117 (1987)). Comparisons among sequences of the AHAS genes in higher plants indicates a high level of conservation of certain regions of the sequence; specifically, there are at least 10 regions of sequence conservation. It has previously been assumed that these conserved regions are critical to the function of the enzyme, and that retention of that function is dependent upon substantial sequence conservation. Therefore, the present invention contemplates overexpression of AHAS in plants to increase the level of Ile and one or more of Arg, Asn, Asp, His, Met, Ala, Leu, Thr, Val, Gln, Tyr, Lys, Ser, and Phe therein.

Aspartate kinase (AK) is the enzyme that catalyzes the first step in the biosynthesis of threonine, isoleucine, lysine, and methionine. Biosynthesis of the aspartate family of amino acids in plants occurs in the plastids, (see, Bryan (1980) In: The Biochemistry of Plants, Vol. 5, B. Miflin (Ed.) Academic Press, N.Y., p. 403). Overexpression of a threonine deregulated has previously been shown to increase in the intracellular levels of free L-threonine in the leaf by 55% (Shaul and Galili, Plant Physiol., 100:1157 (1992)), and in the seed by 15-fold (Karchi et al., Plant J., 3:721(1993)).

Overexpression of either a wild type or deregulated aspartate kinase will increase the available pools of free threonine in the plastids. When combined with overexpression of a wild type, mutant, or deregulated threonine deaminase the amount of threonine converted to isoleucine is increased. In addition to aspartate kinase (AK), homoserine dehydrogenase (HSD) and threonine synthase can be used to increase further the levels of free threonine.

Deregulated aspartate kinases useful in the present invention can possess a level of threonine insensitivity such that at the Km concentration of aspartate in the presence of 0.1 mM threonine, the aspartate kinase enzyme exhibit greater than 10% activity relative to assay conditions in which threonine is absent. Deregulated homoserine dehydrogenases useful in the present invention preferably possess a level of threonine insensitivity such that at 0.1 mM threonine and the Km concentration of aspartate semialdehyde, the enzymes exhibit greater than 10% activity relative to assay conditions in which threonine is absent. The Vmax values for the aspartate kinase and homoserine dehydrogenase enzymes can fall within the range of 0.1-100 times that of their corresponding wild type enzymes. The Km values for the aspartate kinase and homoserine dehydrogenase enzymes can fall within the range of 0.01-10 times that of their corresponding wild type enzymes.

Threonine synthase, the enzyme responsible for converting phosphohomoserine to threonine, has been shown to enhance the level of threonine about 10-fold over the endogenous level when overexpressed in Methylobacillus glycogenes (Motoyama et al., Appl. Microbiol. Biotech., 42:67 (1994)). In addition, E. coli threonine synthase overexpressed in tobacco cell culture resulted in a 10-fold enhanced level of threonine from a 6-fold increase in total threonine synthase activity (Muhitch, Plant Physiol., 108 (2 Suppl.):71 (1995)). Therefore, the present invention contemplates overexpression of threonine synthase in plants to increase the level of threonine therein. This can be employed in the present invention to insure an enhanced supply of threonine for Ile and one or more of Arg, Asn, Asp, His, Met, Ala, Leu, Thr, Val, Gln, Tyr, Lys, Ser, and Phe production by threonine deaminase.

Transformation of Host Cells

A transgene comprising a gene of interest, e.g., a threonine deaminase gene, can be subcloned into a known expression vector, and threonine deaminase expression can be detected and/or quantified. This method of screening is useful to identify expression of a threonine deaminase gene, and expression of a threonine deaminase in the plastid of a transformed plant cell.

Plasmid vectors include additional nucleic acids that provide for easy selection, amplification, and transformation of the transgene in prokaryotic and eukaryotic cells, e.g., pUC-derived vectors such as pUC8, pUC9, pUC18, pUC19, pUC23, pUC119, and pUC120, pSK-derived vectors, pGEM-derived vectors, pSP-derived vectors, or pBS-derived vectors. The additional nucleic acids include origins of replication to provide for autonomous replication of the vector in a bacterial host, selectable marker genes, preferably encoding antibiotic or herbicide resistance, unique multiple cloning sites providing for multiple sites to insert nucleic acids or genes encoded in the transgene, and sequences that enhance transformation of prokaryotic and eukaryotic cells.

Another vector that is useful for expression in both plant and prokaryotic cells is the binary Ti plasmid, as disclosed by Schilperoort et al., U.S. Pat. No. 4,940,838, as exemplified by vector pGA582. This binary Ti plasmid vector has been previously characterized by An, cited supra. This binary Ti vector can be replicated in prokaryotic bacteria such as E. coli or Agrobacterium. The Agrobacterium plasmid vectors can also be used to transfer the transgene to plant cells. The binary Ti vectors preferably include the nopaline T DNA right and left borders to provide for efficient plant cell transformation, a selectable marker gene, unique multiple cloning sites in the T border regions, the colE1 replication of origin and a wide host range replicon. The binary Ti vectors carrying a transgene of the present invention can be used to transform both prokaryotic and eukaryotic cells, but is preferably used to transform plant cells. See, for example, Glassman et al., U.S. Pat. No. 5,258,300.

The expression vector can then be introduced into prokaryotic or eukaryotic cells by available methods. Methods of transformation especially effective for dicots, include, but are not limited to, microprojectile bombardment of immature embryos (U.S. Pat. No. 5,990,390) or Type II embryogenic callus cells as described by W. J. Gordon-Kamm et al., Plant Cell, 2:603 (1990); M. E. Fromm et al., Bio/Technology, 8:833 (1990); and D. A. Walters et al., Plant Molecular Biology, 18:189 (1992), or by electroporation of type I embryogenic calluses described by D'Halluin et al., The Plant Cell, 4:1495 (1992); or by Krzyzek, U.S. Pat. No. 5,384,253. Transformation of plant cells by vortexing with DNA-coated tungsten whiskers (Coffee et al., U.S. Pat. No. 5,302,523) and transformation by exposure of cells to DNA-containing liposomes can also be used.

Strategy for Selection of Isoleucine Overproducer Cell Lines

Efficient selection of a desired isoleucine analog resistant, isoleucine overproducer variant using tissue culture techniques requires careful determination of selection conditions. These conditions are optimized to allow growth and accumulation of isoleucine or isoleucine analog resistant, isoleucine overproducer cells in the culture while inhibiting the growth of the bulk of the cell population. The situation is complicated by the fact that the vitality of individual cells in a population can be highly dependent on the vitality of neighboring cells.

Conditions under which cell cultures are exposed to isoleucine or an isoleucine analog are determined by the characteristics of the interaction of the compound with the tissue. Such factors as the degree of toxicity and the rate of inhibition should be considered. The accumulation of the compounds by cells in culture, and the persistence and stability of the compounds, both in the media and in the cells, also needs to be considered.

The effects of isoleucine or the isoleucine analog on culture viability and morphology is carefully evaluated. It is especially important to choose analog exposure conditions that have no impact on plant regeneration capability of cultures. Choice of analog exposure conditions is also influenced by whether the analog kills cells or simply inhibits cell divisions.

The choice of a selection protocol is dependent upon the considerations described above. The protocols briefly described below may be utilized in the selection procedure. For example, to select for cells that are resistant to growth inhibition by isoleucine or an analog thereof, finely divided cells in liquid suspension culture can be exposed to high isoleucine or analog levels for brief periods of time. Surviving cells are then allowed to recover and accumulate and are then re-exposed for subsequently longer periods of time. Alternatively, organized partially differentiated cell cultures are grown and subcultured with continuous exposure to initially low levels of free L-isoleucine or an analog thereof. Concentrations are then gradually increased over several subculture intervals. While these protocols can be utilized in a selection procedure, the present invention is not limited to these procedures.

Selection and Characterization of Resistant Cell Lines

Selections are carried out until cells or tissue are recovered which are observed to be growing well in the presence of normally inhibitory levels of isoleucine analogs. These cell “lines” are subcultured several additional times in the presence of one or more isoleucine analogs to remove non-resistant cells and then characterized. The amount of resistance that has been obtained is determined by comparing the growth of these cell lines with the growth of unselected cells or tissue in the presence of various analog concentrations. Stability of the resistance trait of the cultured cells may be evaluated by simply growing the selected cell lines in the absence of an analog for various periods of time and then analyzing growth after re-exposing the tissue to the analog. The resistant cell lines may also be evaluated using in vitro chemical studies to verify that the site of action of the analog is within threonine deaminase and/or whether and what mutation has formed to confer less sensitivity to inhibition by isoleucine analog(s).

Transient expression of a threonine deaminase gene can be detected and quantified in the transformed cells. Gene expression can be quantified by reverse transcriptase polymerase chain reaction (RT-PCR) analysis, quantitative Western blot analysis using antibodies specific for the cloned threonine deaminase or by detecting enzyme activity in the presence of isoleucine or an amino acid analog of isoleucine. The tissue and subcellular location of the cloned threonine deaminase can be determined by immunochemical staining methods using antibodies specific for the cloned threonine deaminase or subcellular fractionation and subsequent biochemical and/or immunological analyses. Sensitivity of the cloned threonine deaminase to agents can also be assessed. Transgenes providing for expression of a threonine deaminase or threonine deaminase tolerant to inhibition by an amino acid analog of isoleucine or free L-isoleucine can then be used to transform monocot and/or dicot plant tissue cells and to regenerate transformed plants and seeds. Transformed cells can be selected for the presence of a selectable marker gene or a reporter gene, such as by herbicide resistance. Transient expression of a threonine deaminase gene can be detected in the transgenic embryogenic calli using antibodies specific for the cloned threonine deaminase, or by RT-PCR analyses.

Genes for Plant Modification

As described hereinabove, genes that function as selectable marker genes and reporter genes can be operably combined with the nucleic acid encoding the threonine deaminase, or domain thereof, in transgenes, vectors, and plants of the present invention. Additionally, other agronomical traits can be added to the transgenes, vectors, and plants of the present invention. Such traits include, but are not limited to, insect resistance or tolerance; disease resistance or tolerance (viral, bacterial, fungal, nematode); stress resistance or tolerance, as exemplified by resistance or tolerance to drought, heat, chilling, freezing, excessive moisture, salt stress, oxidative stress; increased yields; food content and makeup; physical appearance; male sterility; drydown; standability; prolificacy; starch properties; oil quantity and quality; and the like. One may incorporate one or more genes conferring such traits into the plants of the present invention.

Environmental or Stress Resistance or Tolerance

Improvement of a plant's ability to tolerate various environmental stresses can be effected through expression of genes. For example, increased resistance to freezing temperatures may be conferred through the introduction of an “antifreeze” protein such as that of the Winter Flounder (Cutler et al., J Plant Physiol., 135:351 (1989)) or synthetic gene derivatives thereof. Improved chilling tolerance may also be conferred through increased expression of glycerol-3-phosphate acetyltransferase in plastids (Wolter et al., EMBO J., 11:4685 (1992)). Resistance to oxidative stress can be conferred by expression of superoxide dismutase (Gupta et al., Proc. Natl. Acad. Sci. (U.S.A.), 90:1629 (1993)), and can be improved by glutathione reductase (Bowler et al., Ann Rev. Plant Physiol., 43:83 (1992)).

It is contemplated that the expression of genes that favorably affect plant water content, total water potential, osmotic potential, and turgor will enhance the ability of the plant to tolerate drought and will therefore be useful. It is proposed, for example, that the expression of genes encoding for the biosynthesis of osmotically active solutes may impart protection against drought. Within this class are genes encoding for mannitol dehydrogenase (Lee and Saier, J. Bacteriol., 258:10761 (1982)) and trehalose-6-phosphate synthase (Kaasen et al., J. Bacteriol., 174:889 (1992)).

Similarly, other metabolites may protect either enzyme function or membrane integrity (Loomis et al., J. Expt. Zoology, 252:9 (1989)), and therefore expression of genes encoding for the biosynthesis of these compounds might confer drought resistance in a manner similar to or complimentary to mannitol. Other examples of naturally occurring metabolites that are osmotically active and/or provide some direct protective effect during drought and/or desiccation include fructose, erythritol, sorbitol, dulcitol, glucosylglycerol, sucrose, stachyose, raffinose, proline, glycine, betaine, ononitol, and pinitol. See, e.g., U.S. Pat. No. 6,281,411.

Three classes of Late Embryogenic Proteins have been assigned based on structural similarities (see, Dure et al., Plant Molecular Biology, 12:475 (1989)). Expression of structural genes from all 3 LEA groups may confer drought tolerance. Other types of proteins induced during water stress, which may be useful, include thiol proteases, aldolases, and transmembrane transporters, which may confer various protective and/or repair-type functions during drought stress. See, e.g., PCT/CA99/00219 (Na+/H+ exchanger polypeptide genes). Genes that effect lipid biosynthesis might also be useful in conferring drought resistance.

The expression of genes involved with specific morphological traits that allow for increased water extractions from drying soil may also be useful. The expression of genes that enhance reproductive fitness during times of stress may also be useful. It is also proposed that expression of genes that minimize kernel abortion during times of stress would increase the amount of grain to be harvested and hence be of value.

Enabling plants to utilize water more efficiently, through the introduction and expression of genes, may improve the overall performance even when soil water availability is not limiting. By introducing genes that improve the ability of plants to maximize water usage across a full range of stresses relating to water availability, yield stability, or consistency of yield performance may be realized.

Plant Composition or Quality

The composition of the plant may be altered, for example, to improve the balance of amino acids in a variety of ways including elevating expression of native proteins, decreasing expression of those with poor composition, changing the composition of native proteins, or introducing genes encoding entirely new proteins possessing superior composition. See, e.g., U.S. Pat. No. 6,160,208 (alteration of seed storage protein expression). The introduction of genes that alter the oil content of the plant may be of value. See, e.g., U.S. Pat. Nos. 6,069,289 and 6,268,550 (ACCase gene). Genes may be introduced that enhance the nutritive value of the starch component of the plant, for example by increasing the degree of branching, resulting in improved utilization of the starch in cows by delaying its metabolism.

Plant Agronomic Characteristics

Two of the factors determining where plants can be grown are the average daily temperature during the growing season and the length of time between frosts. Expression of genes that are involved in regulation of plant development may be useful, e.g., the liguleless and rough sheath genes that have been identified in corn.

Genes may be introduced into corn that would improve standability and other plant growth characteristics. Expression of genes that confer stronger stalks, improved root systems, or prevent or reduce ear droppage, would be of value to the farmer.

Nutrient Utilization

The ability to utilize available nutrients may be a limiting factor in growth of plants. It may be possible to alter nutrient uptake, tolerate pH extremes, mobilization through the plant, storage pools, and availability for metabolic activities by the introduction of genes. These modifications would allow a plant to more efficiently utilize available nutrients. For example, an increase in the activity of an enzyme that is normally present in the plant and involved in nutrient utilization may increase the availability of a nutrient. An example of such an enzyme would be phytase.

Male Sterility

Male sterility is useful in the production of hybrid seed, and male sterility may be produced through expression of genes. It may be possible through the introduction of TURF-13 via transformation to separate male sterility from disease sensitivity. See, Levings, (Science, 250:942-947, (1990)). As it may be necessary to restore male fertility for breeding purposes and for grain production, genes encoding restoration of male fertility, may also be introduced.

Plant Regeneration and Production of Seed

Transformed embryogenic calli, meristemate tissue, embryos, leaf discs, and the like can be used to generate transgenic plants that exhibit stable inheritance of the transformed threonine deaminase gene. Plant cell lines exhibiting satisfactory levels of tolerance to an amino acid analog of isoleucine or free L-isoleucine are put through a plant regeneration protocol to obtain mature plants and seeds expressing the tolerance traits by methods known in the art (for example, see, U.S. Pat. Nos. 5,990,390 and 5,489,520; and Laursen et al., Plant Mol. Biol., 24:51 (1994)). The plant regeneration protocol allows the development of somatic embryos and the subsequent growth of roots and shoots.

To determine that the tolerance trait is expressed in differentiated organs of the plant, and not solely in undifferentiated cell culture, regenerated plants can be assayed for the levels of Ile and one or more of Arg, Asn, Asp, His, Met, Ala, Leu, Thr, Val, Gln, Tyr, Lys, Ser, and Phe present in various portions of the plant relative to regenerated, non-transformed plants. Transgenic plants and seeds can be generated from transformed cells and tissues showing a change in Ile and one or more of Arg, Asn, Asp, His, Met, Ala, Leu, Thr, Val, Gln, Tyr, Lys, Ser, and Phe content or in resistance to a isoleucine analog using standard methods. It is especially preferred that the Ile and one or more of Arg, Asn, Asp, His, Met, Ala, Leu, Thr, Val, Gln, Tyr, Lys, Ser, and Phe content of the leaves or seeds is increased. A change in specific activity of the enzyme in the presence of inhibitory amounts of isoleucine or an analog thereof can be detected by measuring enzyme activity in the transformed cells as described by Widholm, Biochimica et Biophysica Acta, 279:48 (1972). A change in total Ile and one or more of Arg, Asn, Asp, His, Met, Ala, Leu, Thr, Val, Gln, Tyr, Lys, Ser, and Phe content can also be examined by standard methods such as those described by Jones et al., Analyst, 106:968 (1981).

Mature plants are then obtained from cell lines that are known to express the trait. If possible, the regenerated plants are self-pollinated. In addition, pollen obtained from the regenerated plants is crossed to seed grown plants of agronomically important inbred lines. In some cases, pollen from plants of these inbred lines is used to pollinate regenerated plants. The trait is genetically characterized by evaluating the segregation of the trait in first and later generation progeny. The heritability and expression in plants of traits selected in tissue culture are of particular importance if the traits are to be commercially useful.

The commercial value of Ile and one or more of Arg, Asn, Asp, His, Met, Ala, Leu, Thr, Val, Gln, Tyr, Lys, Ser, and Phe overproduction in soybeans, other legumes, cereals, and other plants is greatest if many different hybrid combinations are available for sale. The farmer typically grows more than one kind of hybrid based on such differences as maturity, standability, or other agronomic traits. Additionally, hybrids adapted to one part of the country are not adapted to another part because of differences in such traits as maturity, disease, and insect resistance. Because of this, it is necessary to breed Ile and one or more of Arg, Asn, Asp, His, Met, Ala, Leu, Thr, Val, Gln, Tyr, Lys, Ser, and Phe overproduction into a large number of parental inbred lines so that many hybrid combinations can be produced.

A conversion process (backcrossing) is carried out by crossing the original overproducer line to normal elite lines and then crossing the progeny back to the normal parent. The progeny from this cross will segregate such that some plants carry the gene responsible for overproduction whereas some do not. Plants carrying such genes will be crossed again to the normal parent resulting in progeny that segregate for overproduction and normal production once more. This is repeated until the original normal parent has been converted to an overproducing line, yet possesses all other important attributes as originally found in the normal parent. A separate backcrossing program is implemented for every elite line that is to be converted to Ile and one or more of Arg, Asn, Asp, His, Met, Ala, Leu, Thr, Val, Gln, Tyr, Lys, Ser, and Phe overproducer line.

Subsequent to the backcrossing, the new overproducer lines and the appropriate combinations of lines that make good commercial hybrids are evaluated for overproduction as well as a battery of important agronomic traits. Overproducer lines and hybrids are produced that are true to type of the original normal lines and hybrids. This requires evaluation under a range of environmental conditions where the lines or hybrids will generally be grown commercially. For production of high Ile and one or more of Arg, Asn, Asp, His, Met, Ala, Leu, Thr, Val, Gln, Tyr, Lys, Ser, and Phe soybeans, it may be necessary that both parents of the hybrid seed be homozygous for the high Ile and one or more of Arg, Asn, Asp, His, Met, Ala, Leu, Thr, Val, Gln, Tyr, Lys, Ser, and Phe character. Parental lines of hybrids that perform satisfactorily are increased and used for hybrid production using standard hybrid seed production practices.

The transgenic plants produced herein are expected to be useful for a variety of commercial and research purposes. Transgenic plants can be created for use in traditional agriculture to possess traits beneficial to the consumer of the grain harvested from the plant (e.g., improved nutritive content in human food or animal feed). In such uses, the plants are generally grown for the use of their grain in human or animal foods. However, other parts of the plants, including stalks, husks, roots, tubers, flowers, vegetative parts, and the like, may also have utility, including use as part of animal silage, fermentation feed, biocatalysis, or for ornamental purposes.

Transgenic plants may also find use in the commercial manufacture of proteins or other molecules, where the molecule of interest is extracted or purified from plant parts, seeds, and the like. Cells or tissue from the plants may also be cultured, grown in vitro, or fermented to manufacture such molecules.

The transgenic plants may also be used in commercial breeding programs, or may be crossed or bred to plants of related crop species. Improvements encoded by the recombinant DNA may be transferred, e.g., from soybean cells to cells of other species, e.g., by protoplast fusion.

The following examples are provided to further illustrate certain aspects of the present invention.

### EXAMPLE 1

This example sets forth the construction of plant expression vectors containing polynucleotide allelic variants that encode threonine deaminase enzymes.

In particular, amino acid L481 was selected for rational design of a deregulated threonine deaminase. Several mutant alleles were generated each having higher or lower IC₅₀^{Ile} values than the ilvA L481F variant allele. These alleles were used to determine the range of feedback insensitivity for threonine deaminase for use in transgenic plants. Table 2 (above) lists the amino acid substitutions made in ilvA at amino acid position 481.

In the examples described herein, DNA modifying enzymes including restriction enzymes were purchased from New England Biolabs (Beverly, Mass.). Oligonucleotide primers were synthesized by Invitrogen Life Technologies (Carlsbad, Calif.). All other chemicals were purchased from Sigma-Aldrich (St Louis, Mo.). Protein determinations were performed as described (Bradford, Anal. Biochem., 72:248-254 (1976)).

The ilvA alleles used were derived from the wild type E. coli ilvA threonine deaminase gene (SEQ ID NO: 1), which encodes SEQ ID NO: 2 that was available in the GenBank database (accession number K03503; Lawther et al., Nucleic Acids Res., 15:2137 (1987)). Isoleucine-deregulated threonine deaminase variants were generated by mutagenesis of E. coli and isolated as described (Gruys et al., U.S. Pat. No. 5,942,660; Asrar et al., U.S. Pat. Nos. 6,091,002 and 6,228,623; and Slater et al., Nature Biotechnology, 7:1011-1016 (1999)). The nucleotide sequence of the mutagenized E. coli threonine deaminase gene containing the ilvA219 (L447F) mutation is SEQ ID NO: 14 and its respective translated polypeptide sequence is SEQ ID NO: 3. The nucleotide sequence of the mutagenized E. coli threonine deaminase gene containing the ilvA466 (L481F) mutation is SEQ ID NO: 15. All mutations were confirmed by DNA sequence analysis.

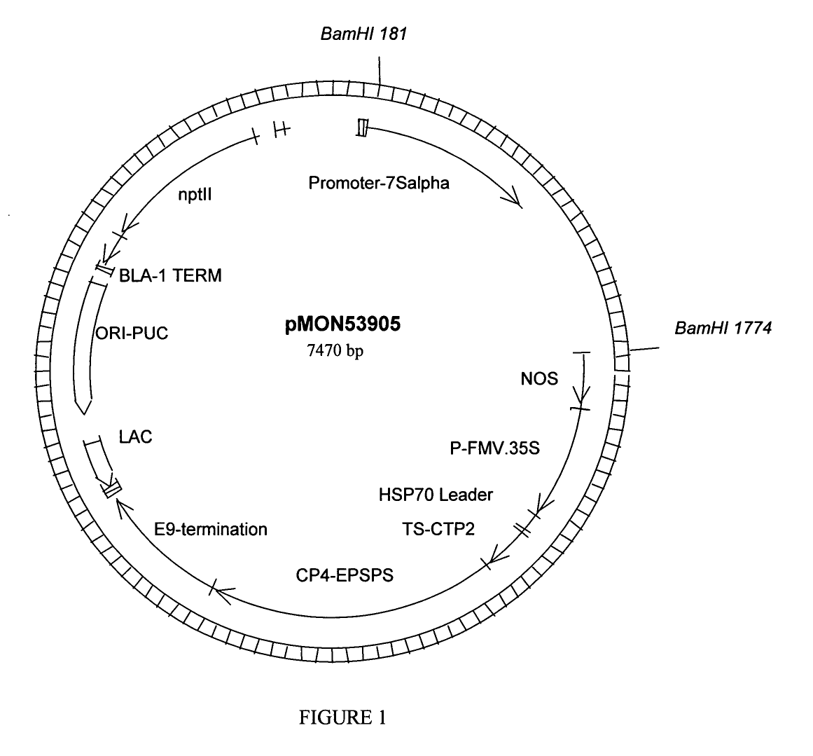

The plasmid pMON53905 (FIG. 1) was digested with the restriction enzyme BamH1 to generate a 5.9 Kbp backbone fragment. This fragment served as the common backbone fragment for the constructs described below.

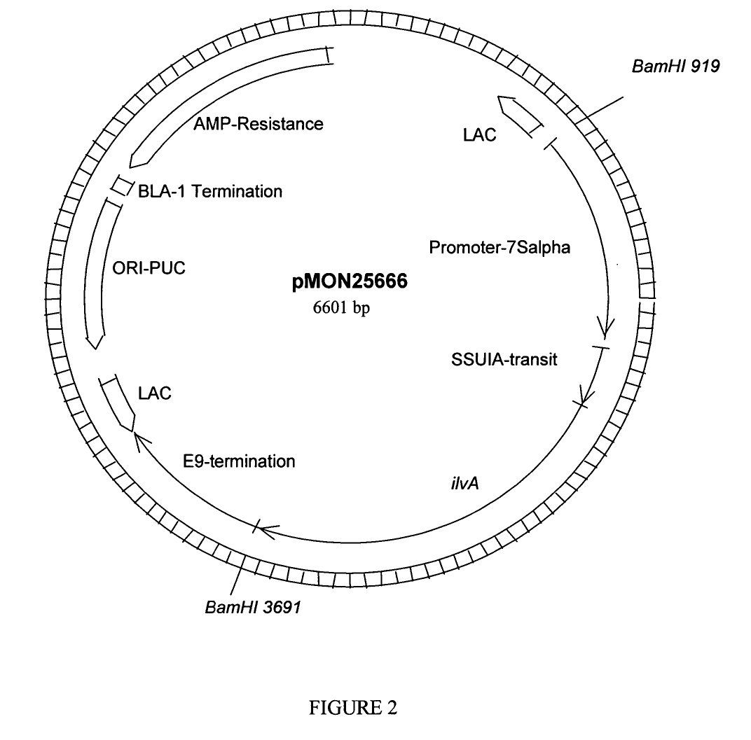

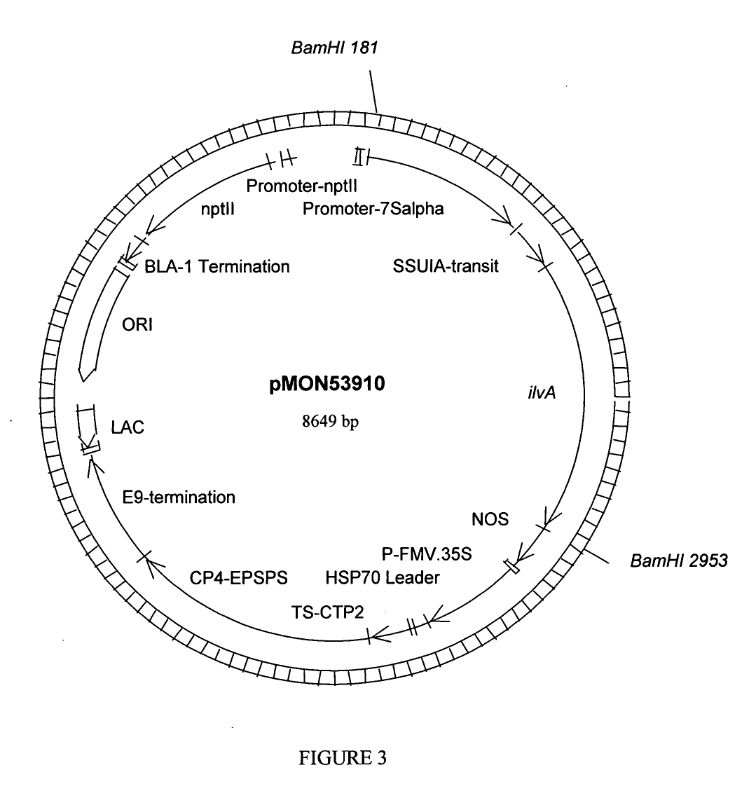

Plasmid pMON25666 (FIG. 2) was digested with BamH1 to generate 2 fragments of 3.8 and 2.8 Kbp. The 2.8 Kbp fragment was then ligated into the 5.9 Kbp backbone fragment from pMON53905 to generate the plasmid named pMON53910 (FIG. 3). This plasmid contained the wild type ilvA gene (SEQ ID NO: 1) and served as a control.

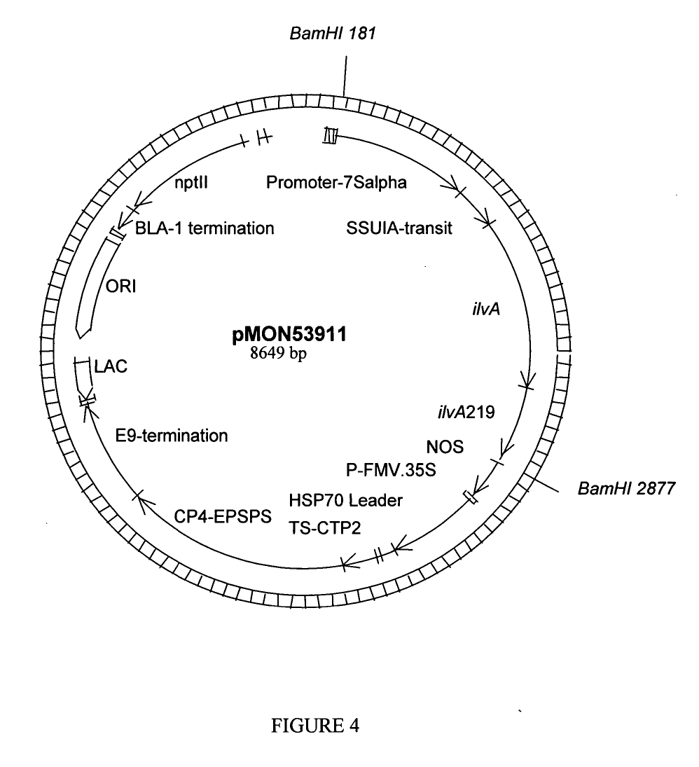

Plasmid pMON25694 was digested with BamH1 to generate 2 fragments of 3.8 and 2.8 Kbp. The 2.8 Kbp fragment was then ligated into the 5.9 Kbp backbone fragment (from pMON53905) to generate the plasmid named pMON53911 (FIG. 4). This plasmid contained the mutagenized E. coli threonine deaminase gene, ilvA219 (M447F) (SEQ ID NO: 14).

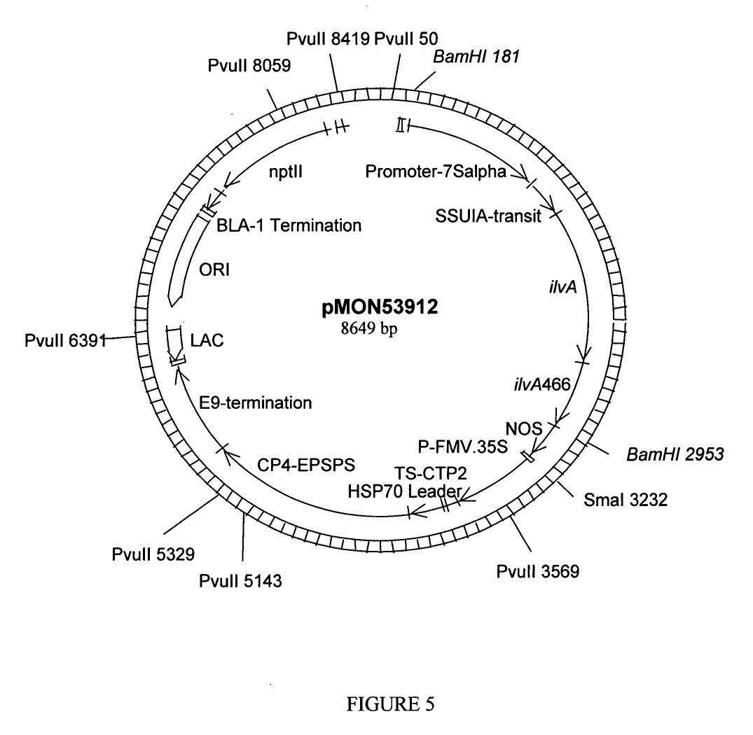

Plasmid pMON25695 was digested with BamH1 to generate 2 fragments of 3.8 and 2.8 Kbp. The 2.8 Kbp fragment was then ligated into the 5.9 Kbp backbone fragment to generate the plasmid named pMON53912 (FIG. 5). This plasmid contained the mutagenized E. coli biosynthetic threonine deaminase gene, ilvA466 (L481F) (SEQ ID NO: 15).

### EXAMPLE 2

Before conducting further transformation experiments using the isolated ilvA alleles in transgenic plants, each allele was over-expressed in E. coli to determine its kinetic parameters. Kinetics data on threonine deaminases containing various mutations, and a comparison to data available for threonine deaminases from Arabidopsis, are provided in Table 4. The E. coli ilvA481 variants were subcloned into pSE380 (Invitrogen, Carlsbad, Calif.) and expression was induced with 0.2 mM IPTG at 37° C. for 3 hours. Expression of the E. coli alleles was high and fairly consistent as visualized by SDS-PAGE. Each variant threonine deaminase accounted for greater than 50% of the total soluble protein in E. coli. The only exception was the L481K variant threonine deaminase, which had poor expression and poor enzyme activity.

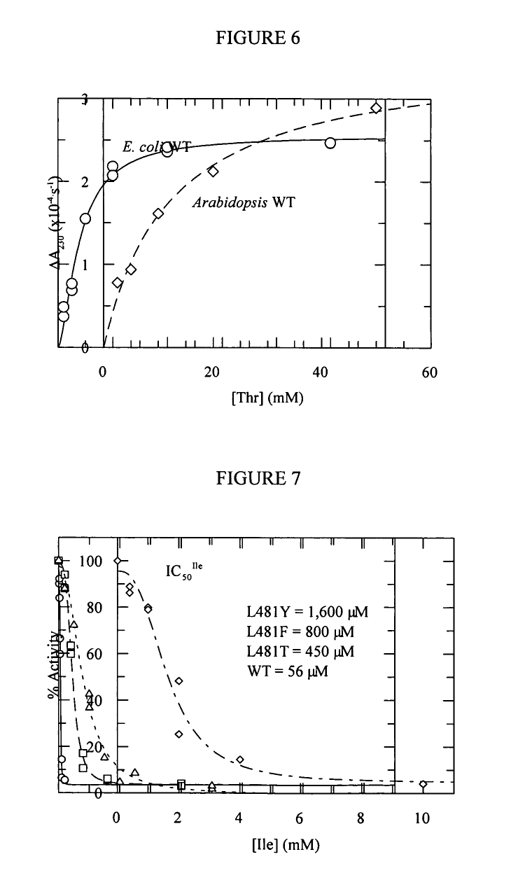

The effects of amino acid substitutions at Leu481 in ilvA were assessed by steady state kinetic analysis in the presence and absence of L-isoleucine. Threonine deaminase polypeptides for use in in vitro kinetics studies were extracted from E. coli cells in assay buffer containing 50 mM potassium phosphate (pH7.5), 1 mM dithiothreitol (DTT), and 0.5 mM ethylenediamine-tetraacetate. A continuous assay method was employed to monitor the formation of α-ketobutyrate directly at 230 nm (ε₂₃₀ (pH7.5)=540 M⁻¹cm⁻¹ whereas threonine absorption was negligible (˜1%)). The assay was initiated by adding 20 μl of crude extract diluted 1:20 v/v to the assay vessel containing L-threonine (between 2.5 mM and 50 mM) in a final volume of 1 mL. For L-isoleucine inhibition, L-isoleucine was added between 0 mM and 20 mM. The kinetic parameters were determined by fitting the data points to the equations using GraFit 4.0 software (Erithacus Software, Surrey, UK). For comparison, the k_{cat} values of L481 alleles were normalized to the k_{cat} value for the wild type IlvA enzyme. The results of these analyses are provided in FIGS. 6 and 7. Enzymes represented in FIG. 7 are: wild type E. coli threonine deaminase (circles), L481Y TD enzyme (diamonds), L481F TD enzyme (triangles), and the L481T TD enzyme (squares). Table 4 also summarizes the kinetic parameters of the variant threonine deaminase enzymes produced by the various E. coli ilvA alleles.

TABLE 4
Kinetics data for certain threonine deaminases expressed in E. coli.

| | | Kₘ^{Thr} | |
|---|---|---|---|
| pMON | TD Polypeptide | (mM) | IC₅₀^{Ile} (μM) |
| NA | Wild type Arabidopsis | 2.8 | 10 |
| NA | Mutant Arabidopsis (OMR1) | 3.6 | 500 |
| 25858 | E. coli (wt) (SEQ ID NO: 2) | 8.3 | 56 |
| 25859 | L447F (ilvA219) (SEQ ID NO: 3) | 1.7 | >20,000 |
| 25857 | L481F (ilvA466) (SEQ ID NO: 4) | 4 | 800 |
| 25868 | L481Y (SEQ ID NO: 5) | 2 | 1,600 |
| 25864 | L481P (SEQ ID NO: 6) | 8.8 | 650 |
| 25860 | L481E (SEQ ID NO: 7) | 3.9 | 445 |
| 25866 | L481T (SEQ ID NO: 8) | 3.4 | 449 |
| 25865 | L481Q (SEQ ID NO: 9) | 8.8 | 188 |
| 25861 | L481I (SEQ ID NO: 10) | 7.6 | 134 |
| 25867 | L481V (SEQ ID NO: 11) | 7.1 | 97 |
| 25863 | L481M (SEQ ID NO: 12) | 6.4 | 100 |

All L481 alleles displayed positive cooperativity (a sigmoidal curve) in substrate binding, whereas Arabidopsis threonine deaminase showed independent activity (a typical hyperbolic curve) (FIG. 6). The degree of cooperativity (Hill coefficient) of the mutants was in the range of 1.1 (pMON25868, L481Y) to 1.6 (pMON25865, L481Q; pMON25861, L481I) (Table 4). Interestingly, a curve of kinetics data for L481Y (n=1.1) fit into a hyperbolic curve with 99% confidence by F-test (JMP statistical software (SAS Institute, Cary, N.C.). In the presence of isoleucine, the activities of L481 mutant enzymes were inhibited with IC₅₀ values ranging from 97 μM (pMON25867, L481V) to 1,600 μM (pMON25868, L481Y) (FIG. 7 and Table 4). None of the L481 mutants compromised the substrate binding affinity (Kₘ) with the greater IC₅₀ values (Table 4). Hence, substrate binding affinity (Kₘ) was comparatively unaffected by mutation of the isoleucine binding pocket at residue 481. Unlike the L481 mutants, the L447F ilvA219 mutant displayed a negative cooperativity (n=0.5) although this mutant was only slightly inhibited by isoleucine (IC₅₀>20,000 μM).

Based on these kinetic data, four L481 alleles, ranging in IC₅₀^{Ile} from 100 μM (L481M) to 1,600 μM (L481Y) were selected for Arabidopsis transformation.

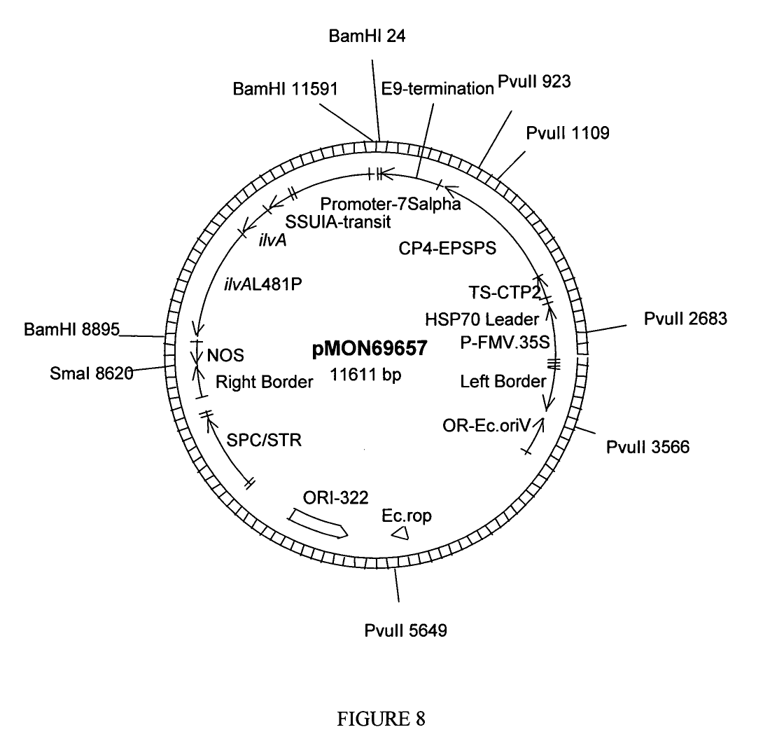

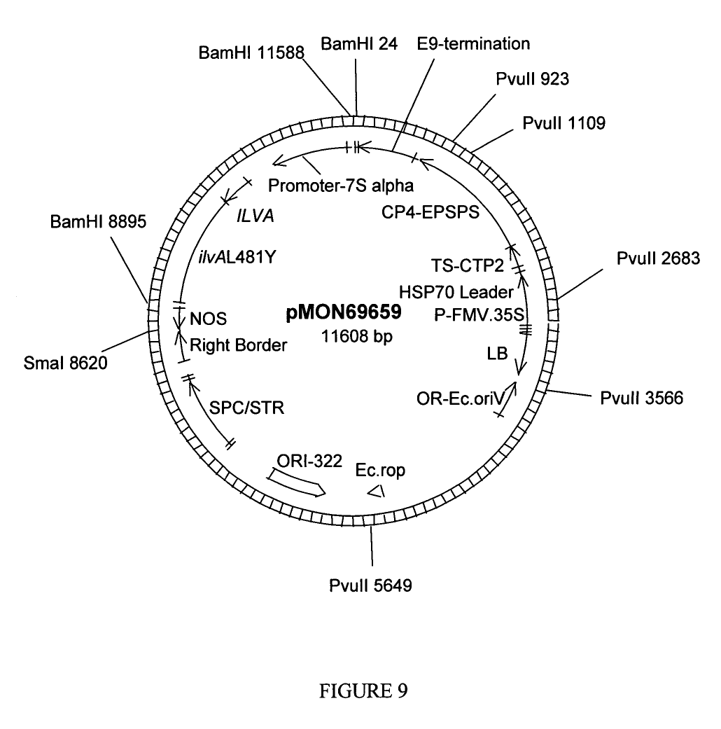

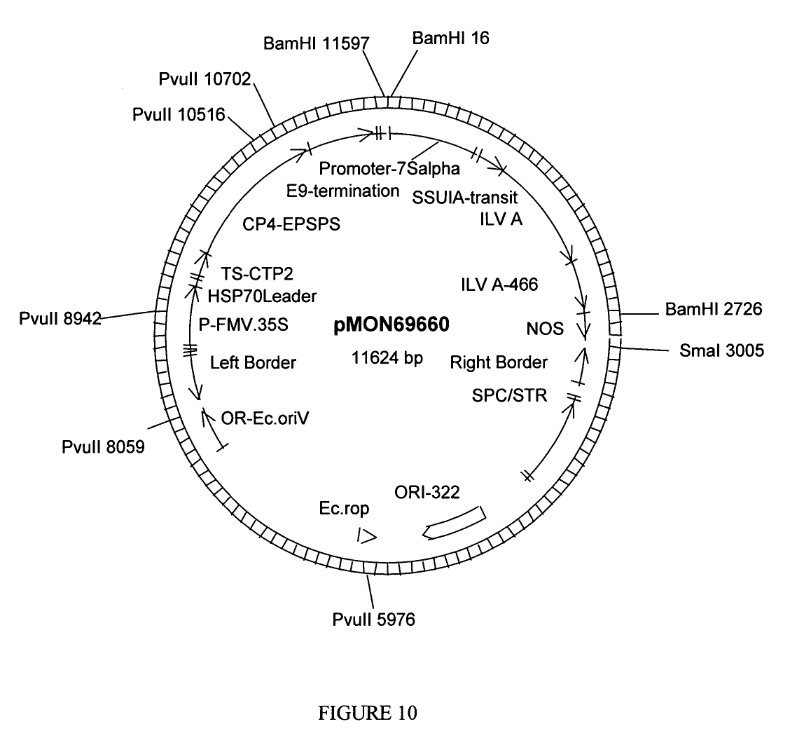

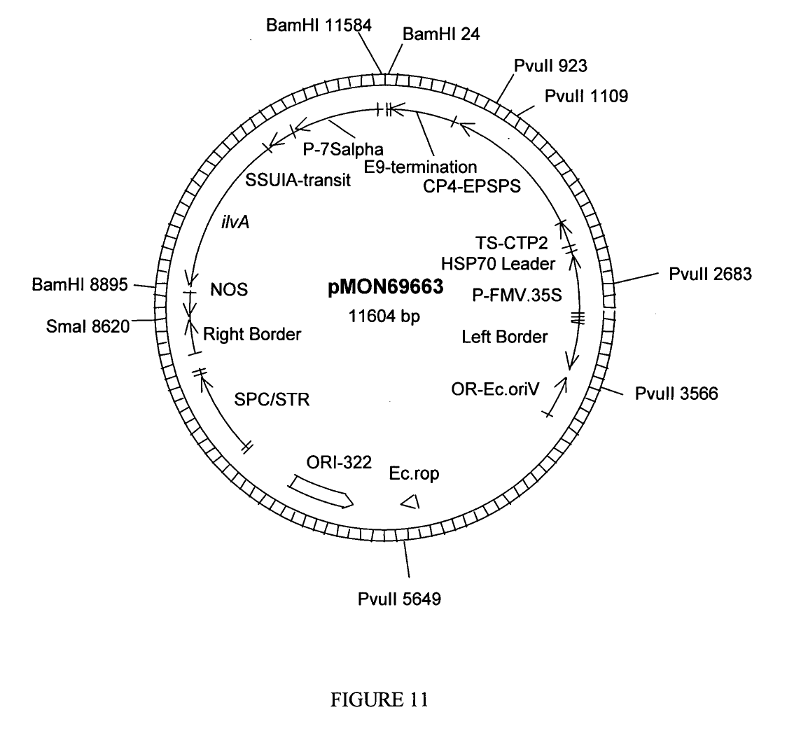

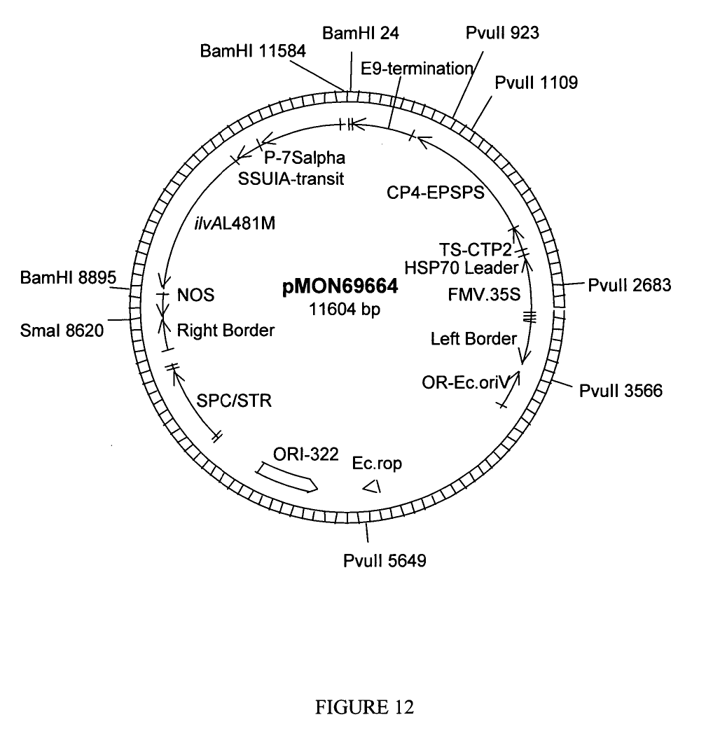

Each L481 allele was then subcloned from the E. coli expression plasmids described in Table 4 into seed specific plant expression plasmids for transformation into Arabidopsis plants. E. coli ilvA481 alleles were excised from the E. coli expression plasmids listed in Table 4 and cloned into an intermediate vector as cassettes containing a seed enhanced promoter (7Sα′; Doyle et al., J. Biol. Chem., 261:9228-9238 (1986)), an open reading frame encoding a Arabidopsis SSU1A transit peptide (Stark et al., Science, 258:287 (1992)) fused to an open reading frame containing one of the five the ilvA481 alleles, and a 3′ untranslated region (NOS; Depicker et al., J. Mol. Appl. Genet., 1(4):361-370 (1982)). The resulting binary plant transformation plasmids pMON69657 (L481P) (FIG. 8), pMON69659 (L481Y) (FIG. 9), pMON69660 (L481F) (FIG. 10), pMON69663 (L481I) (FIG. 11), and pMON69664 (L481M) (FIG. 12) were transformed into Arabidopsis by Agrobacterium mediated infiltration (Beachtold et al., C.R. Acad. Sci. Ser. 111, 316:1194-1199 (1993)). Transformants were selected in the presence of 50 uM glyphosate.

Transformed plant extracts were screened for threonine deaminase activity using the colorimetric endpoint assay (Szamosi et al., Plant Phys., 101:999-1004 (1993)). The endpoint assay was run in reaction buffer containing 100 mM Tris-HCl pH 9.0, 100 mM KCl, 12.5 mM L-threonine. The reaction was initiated by adding 50 μl of enzyme extract to a final volume of 333 μl. Reactions were incubated at 37° C. for 30 minutes and quenched with 333 μl of 0.05% DNPH (dinitrophenylhydrazine) in 1N HCl. This was incubated for 10 minutes at room temperature before neutralizing with 333 μl of 4N NaOH. The reaction products were transferred to disposable cuvettes (Sarstedt) and read at 540 nm using an HP8453 diode array spectrophotometer. Several independent events were generated containing the various L481 alleles. Transformation with pMON69657 (L481P) (FIG. 8) had an unusually low transformation frequency. The low efficiency was attributed to the transformation selection conditions and not the particular threonine deaminase allele employed (data not shown). All surviving plants transformed with the various L481 alleles were phenotypically indistinguishable from the controls and had normal seed set indicating that the expression of the threonine deaminase alleles was not deleterious to the health of the plant.

In order to determine isoleucine concentrations in transformed plants, desiccated, mature Arabidopsis seeds and other vegetative tissues were collected and subjected to standard amino acid analysis. Briefly, 5 mg of non-seed plant tissue was extracted in 100 μL of 5% trichloroacetic acid by vortexing at room temperature for 15 minutes. Extracts were centrifuged at 16,000g for 15 minutes, and the supernatant was transferred to HPLC vials for analysis according to Agilent (Technical Publication, April 2000). Amino acid concentrations were measured by fluorescence spectroscopy at an excitation wavelength of 340 nm and emission of 450 nm.

In order to determine the amino acid concentration in seeds, 20 mg of mature Arabidopsis seed, 500 μl of 0.5 mm zirconium/silica beads (Boise Products, Inc.) and 400 μL of extraction buffer (100 mM potassium phosphate pH 7.4, 5 mM magnesium chloride, 1 mM EGTA, 2 mM DTT, 2 mM 4-2-aminoethyl benzenesulfonyl fluoride (AEBSF), 100 μM leupeptin, 10% glycerol) were aliquoted into 2 mL screw capped vials. Seeds were pulverized at 4° C. for two 45-second runs on a bead beater (Biospec Products, Inc.) at the highest setting. The cell homogenate was centrifuged at 16,000 g for 10 minutes at 4° C. and the supernatant was analyzed by fluorescence spectroscopy at an excitation wavelength of 340 nm and emission of 450 nm.

Table 5A-5B shows the isoleucine accumulation (ppm) in R2 generation seed for pMON69659 (L481Y) (FIG. 9), pMON69660 (L481F) (FIG. 10), pMON69663 (L481I) (FIG. 11), and pMON69664 (L481M) (FIG. 12) events. As expected, there was a wide distribution of isoleucine accumulation in the transgenic plants from different events. Events transformed with pMON69659 (L481Y) produced an average of 85.9±37.4 ppm Ile with a range of 38.1 to 153.9 ppm. Events transformed with pMON69660 (L481F) produced an average of 319.6±397.4 ppm Ile with a range of 41.4 to 2592 ppm. Events transformed with pMON69663 (L481I) produced an average of 204.3±159.1 ppm Ile with a range of 55.4 to 728.2 ppm. Events transformed with pMON69664 (L481M) produced an average of 168.1±232.0 ppm Ile with a range of 42.3 to 1308.6 ppm. Control events that were not transformed with genes encoding threonine deaminase produced an average 73.75±2.5 ppm Ile. One event, 8315, which was based on the L481F (ilvA466) allele, produced a 23-fold increase in Ile, the largest increase observed.

The majority of transformants did not accumulate isoleucine to increase levels relative to controls. Moreover, there did not appear to be any correlation between the IC₅₀^{Ile} and the amount of isoleucine that was accumulated in the transgenic plants. For example, lines transformed with pMON69659 (L481Y) had the highest IC₅₀^{Ile} but did not produce any events with significantly elevated levels of isoleucine.

TABLE 5A
The Ile concentration (ppm) in Arabidopsis plants transformed
with four different threonine deaminase constructs.

| pMON | Event | Ile (ppm) |
|---|---|---|
| NA | Control | 70.0 |
| 69659 | 8263 | 38.1 |
| 69659 | 8284 | 38.3 |
| 69659 | 8275 | 39.0 |
| 69659 | 8261 | 43.5 |
| 69659 | 8277 | 50.9 |
| 69659 | 8271 | 52.0 |
| 69659 | 8262 | 55.6 |
| 69659 | 8265 | 62.3 |
| 69659 | 8266 | 68.2 |
| 69659 | 8279 | 74.7 |
| 69659 | 8276 | 76.2 |
| 69659 | 8286 | 78.2 |
| 69659 | 8269 | 81.3 |
| 69659 | 8268 | 84.7 |
| 69659 | 8270 | 87.5 |
| 69659 | 8278 | 94.6 |
| 69659 | 8258 | 97.6 |
| 69659 | 8287 | 100.4 |
| 69659 | 8264 | 116.7 |
| 69659 | 8260 | 125.0 |
| 69659 | 8259 | 143.4 |
| 69659 | 8272 | 150.1 |
| 69659 | 8273 | 150.8 |
| 69659 | 8274 | 153.9 |
| NA | Control | 75.0 |
| 69660 | 7946 | 41.4 |
| 69660 | 8301 | 92.5 |
| 69660 | 8309 | 102.9 |
| 69660 | 8300 | 116.1 |
| 69660 | 7943 | 118.2 |
| 69660 | 8298 | 119.3 |
| 69660 | 8292 | 128.9 |
| 69660 | 8314 | 136.9 |
| 69660 | 8307 | 139.6 |
| 69660 | 8312 | 151.6 |
| 69660 | 8296 | 164.2 |
| 69660 | 8308 | 167.8 |
| 69660 | 8295 | 174.1 |
| 69660 | 8297 | 189.2 |
| 69660 | 8294 | 198.1 |
| 69660 | 8306 | 198.2 |
| 69660 | 8290 | 205.5 |
| 69660 | 8317 | 218.1 |
| 69660 | 8310 | 224.0 |
| 69660 | 8311 | 236.1 |
| 69660 | 8316 | 258.5 |
| 69660 | 8313 | 265.8 |
| 69660 | 8289 | 324.9 |
| 69660 | 8288 | 336.4 |
| 69660 | 8299 | 346.8 |
| 69660 | 8291 | 403.3 |
| 69660 | 8305 | 451.4 |
| 69660 | 8303 | 485.1 |
| 69660 | 8302 | 540.0 |
| 69660 | 8304 | 590.7 |
| 69660 | 8293 | 809.7 |
| 69660 | 8315 | 2292.0 |

TABLE 5B
The Ile concentration (ppm) in Arabidopsis plants illustrated
transformed with four different threonine deaminase constructs.

| pMON | Event | Ile (ppm) |
|---|---|---|
| NA | Control | 75.0 |
| 69663 | 8452 | 55.4 |
| 69663 | 8459 | 80.0 |
| 69663 | 8453 | 81.7 |
| 69663 | 8445 | 82.2 |
| 69663 | 8443 | 92.1 |
| 69663 | 8447 | 92.1 |
| 69663 | 8465 | 93.9 |
| 69663 | 8444 | 95.5 |
| 69663 | 8467 | 98.4 |
| 69663 | 8450 | 104.2 |
| 69663 | 8460 | 111.4 |
| 69663 | 8442 | 112.9 |
| 69663 | 8439 | 131.7 |
| 69663 | 8463 | 133.4 |
| 69663 | 8451 | 156.6 |
| 69663 | 8457 | 174.4 |
| 69663 | 8441 | 177.4 |
| 69663 | 8438 | 190.9 |
| 69663 | 8461 | 196.1 |
| 69663 | 8455 | 197.8 |
| 69663 | 8446 | 212.9 |
| 69663 | 8458 | 223.8 |
| 69663 | 8456 | 247.4 |
| 69663 | 8449 | 287.6 |
| 69663 | 8448 | 307.5 |
| 69663 | 8440 | 309.1 |
| 69663 | 8466 | 410.2 |
| 69663 | 8464 | 496.5 |
| 69663 | 8454 | 578.1 |
| 69663 | 8462 | 728.2 |
| NA | Control | 75.0 |
| 69664 | 8492 | 42.3 |
| 69664 | 8468 | 44.8 |
| 69664 | 8469 | 47.0 |
| 69664 | 8493 | 53.4 |
| 69664 | 8475 | 62.0 |
| 69664 | 8481 | 64.2 |
| 69664 | 8490 | 78.8 |
| 69664 | 8478 | 85.0 |
| 69664 | 8477 | 86.7 |
| 69664 | 8494 | 90.3 |
| 69664 | 8470 | 94.0 |
| 69664 | 8484 | 98.4 |
| 69664 | 8473 | 100.0 |
| 69664 | 7982 | 114.3 |
| 69664 | 8480 | 119.7 |
| 69664 | 8471 | 125.8 |
| 69664 | 8488 | 126.5 |
| 69664 | 8496 | 135.6 |
| 69664 | 8487 | 140.9 |
| 69664 | 8479 | 141.4 |
| 69664 | 8495 | 150.2 |
| 69664 | 8483 | 183.2 |
| 69664 | 8489 | 183.4 |
| 69664 | 8486 | 184.0 |
| 69664 | 8485 | 187.5 |
| 69664 | 8472 | 197.8 |
| 69664 | 8491 | 220.2 |
| 69664 | 8482 | 502.3 |
| 69664 | 8476 | 1308.6 |

To determine if there was any correlation between the levels of isoleucine produced and the relative expression levels of threonine deaminase, Western blot and enzyme activity analyses were performed on several of the high isoleucine accumulating and low isoleucine accumulation lines. Briefly, approximately 10 μg of soluble crude extract was loaded on 4%-20% gradient SDS-PAGE gels (Zaxis). Protein was transferred to PVDF membranes (Biorad). Blots were blocked with 5% milk in TBST (Tris-buffered saline with 0.05% Tween 20) for 1 hour. The blot was probed with a 1:3000 dilution (using TBST with 0.5% BSA) of rabbit serum (MR324) containing polyclonal antibodies against the purified enzyme for 1 hour. Following probing with anti-rabbit alkaline phosphate conjugated antibodies the membranes were developed using Sigma Fast BCIP/NBT tablets (Sigma, St. Louis, Mo.).

The results indicated that there was no clear correlation between expression, activity, and isoleucine accumulation (data not presented). Activity was only detectable in lines containing the highest levels of threonine deaminase accumulation even though all L481 alleles were shown to accumulate Western positive signals. In order to detect activity in lines with lower expression a more sensitive assay could be used (Gruys et al., 1999).

### EXAMPLE 3

This example sets forth a method for increasing isoleucine and valine concentrations in an Arabidopsis plant by combining an isoleucine-deregulated threonine deaminase (TD) enzyme (ilvA466, SEQ ID NO: 15) with additional enzymes involved in the valine and isoleucine biosynthesis pathway, namely, polynucleotide molecules encoding the E. coli ilvG acetolactate synthase large subunit (EC:2.2.1.6; SEQ ID NO: 16) and the ilvM acetolactate synthase II, small subunit (EC:2.2.1.6; SEQ ID NO:17).

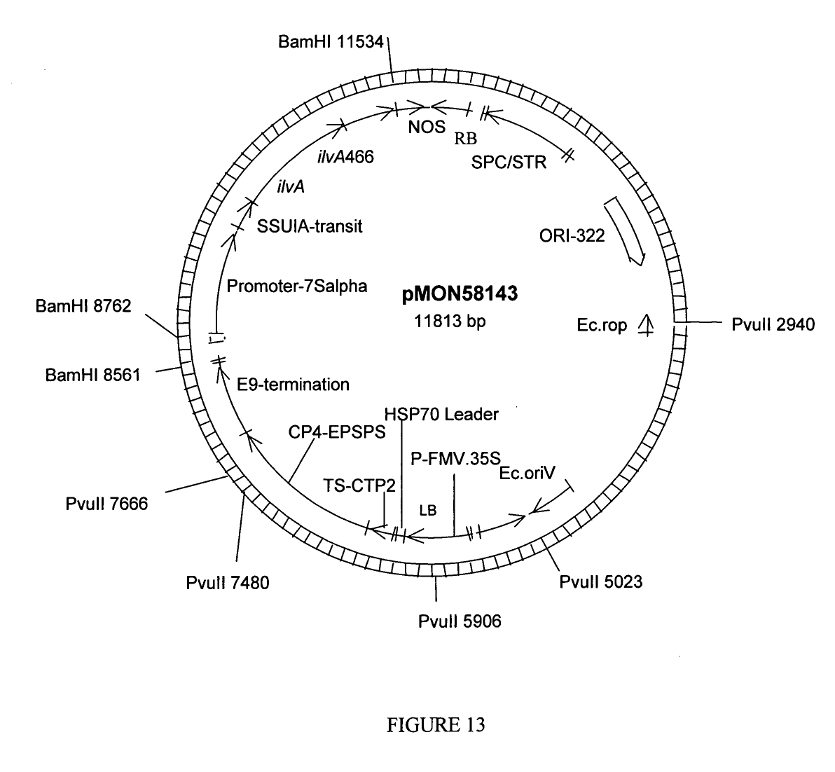

The threonine deaminase E. coli IlvA466 allele (SEQ ID NO: 15) was excised from pMON53912 using SmaI and PvuII restriction enzymes, and ligated into base vector pMON38207 at the SmaI and PmeI restriction sites to create pMON58143. Vector pMON58143 (FIG. 13) was used in Agrobacterium mediated transformation conducted under kanamycin selection.

The genes encoding ilvG and ilvM were isolated by polymerase chain reaction (PCR) using primer pairs based on their respective primary sequences. pMON58131 contains the ilvG gene (SEQ ID NO: 16). SEQ ID NO: 16 was ligated into a pGEM-Teasy vector (Promega Corporation, USA) to make vector TTFAGA018992. A 5′ polynucleotide fragment of the ilvG gene (SEQ ID NO: 18) was excised from TTFAGA018992, using BspH1 and KpnI restriction enzymes, and ligated into an intermediate vector containing the Arabidopsis SSU1A transit peptide (SEQ ID NO: 19; Stark et al., Science, 258:287 (1992)) to create pMON58145. The operably linked SSU1A transit peptide (SEQ ID NO: 19) and ilvG gene fragment (SEQ ID NO: 18) was then excised with KpnI and NcoI restriction enzymes, and ligated into pMON58132. The operably linked SEQ ID NOs: 18 and 19 was then excised from pMON58132, using Bg1II and KpnI restriction enzymes, and ligated into a shuttle vector, pMON36220, excised using SmaI and KpnI restriction enzymes, and ligated into pMON58146. The remaining 3′ ilvG polynucleotide fragment (SEQ ID NO: 20) was excised from TTFAGA018992 using KpnI and EcoRI restriction enzymes, ligated into pMON58146 in operable linkage with SEQ ID NOs: 18 and 19 to create pMON58147. The SSU1A transit peptide (SEQ ID NO: 19) and complete ilvG coding region (SEQ ID NO: 16) were then excised from pMON58147 using NotI and EcoRI restriction enzymes and ligated into pMON64205. The SSU1A transit peptide/ilvG cassette which was in turn excised from pMON64205 using PmeI and Bg1II, was then operably linked to the 7s-alpha promoter (U.S. Publication No. 2003/0093828) and the arcelin 5 3′ untranslated region (WO 02/50295-A2) to create pMON58136. The entire cassette was excised from pMON58136 using NotI and BspHI and ligated into transformation vector pMON38207 to create pMON58138.

pMON58133 contains the ilvM polynucleotide sequence (SEQ ID NO: 17). SEQ ID NO: 17 was ligated into PGEM-Teasy (Promega, supra) to create pMON58137. SEQ ID NO: 17 was then excised from pMON58137 using BspHI and NotI restriction enzymes, and ligated into pMON58129 (previously digested with PmeI and NcoI). This caused SEQ ID NO: 17 to be operably linked to the Napin promoter (U.S. Pat. No. 5,420,034), the Arabidopsis SSU1A transit peptide and the ADR12 3′-untranslated region (U.S. Pat. No. 5,981,841). This plasmid was called pMON58140. The expression cassette was excised using BspHI and NotI restriction enzymes and ligated into the plant transformation vector pMON38207 (previously digested with restriction enzyme NotI) to create pMON58151.

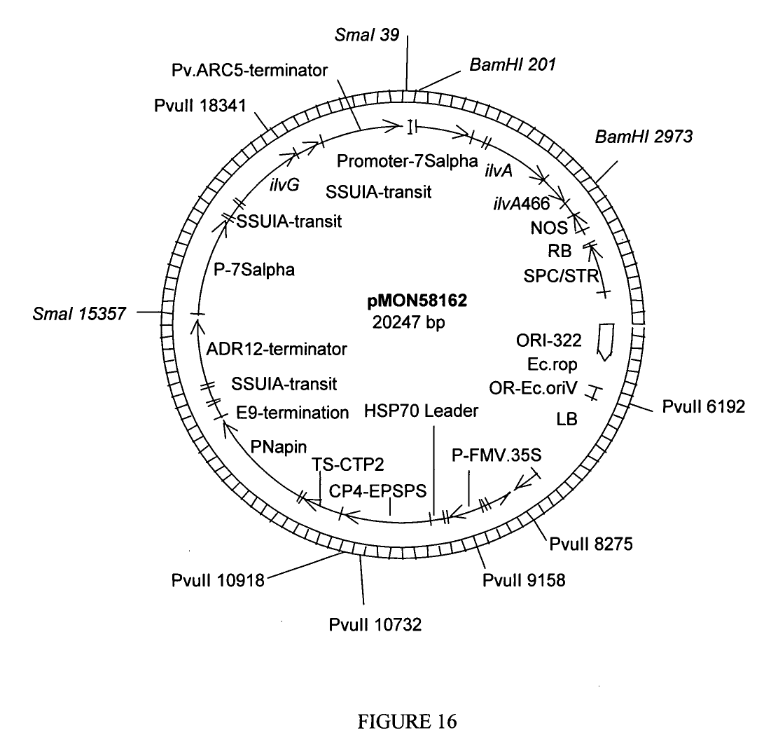

The ilvM cassette was excised from its intermediate vector pMON58140 using NotI and BspHI restriction enzymes, and ligated into pMON58138, which contained the ilvG cassette and plant transformation backbone to create pMON58159. In addition, ilvA466 was excised from pMON53912 using PvuII and SmaI restriction enzymes and operably linked with the ilvG and ilvM cassettes from pMON58159 to create pMON58162 (FIG. 16).

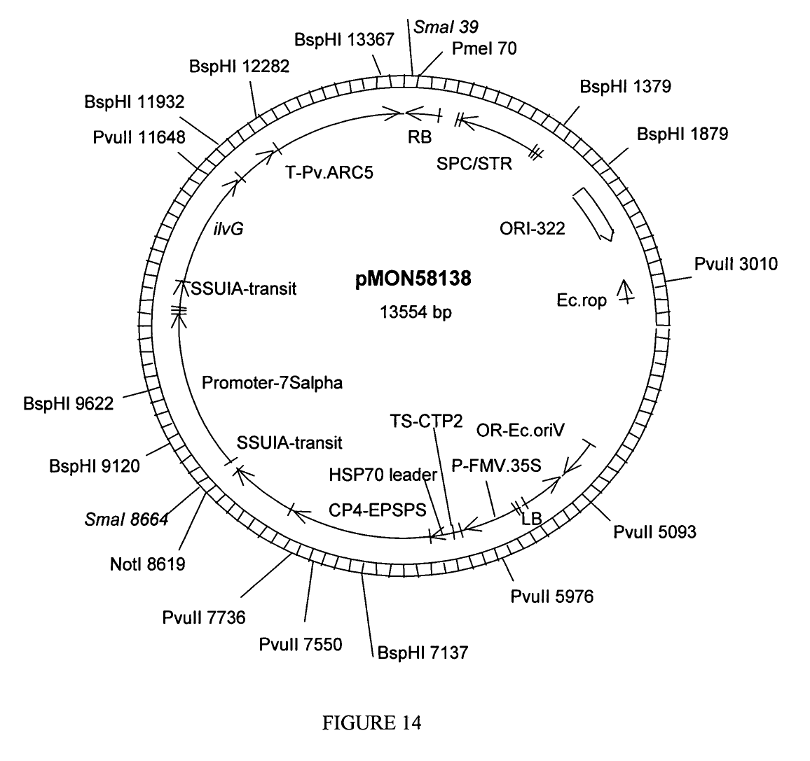

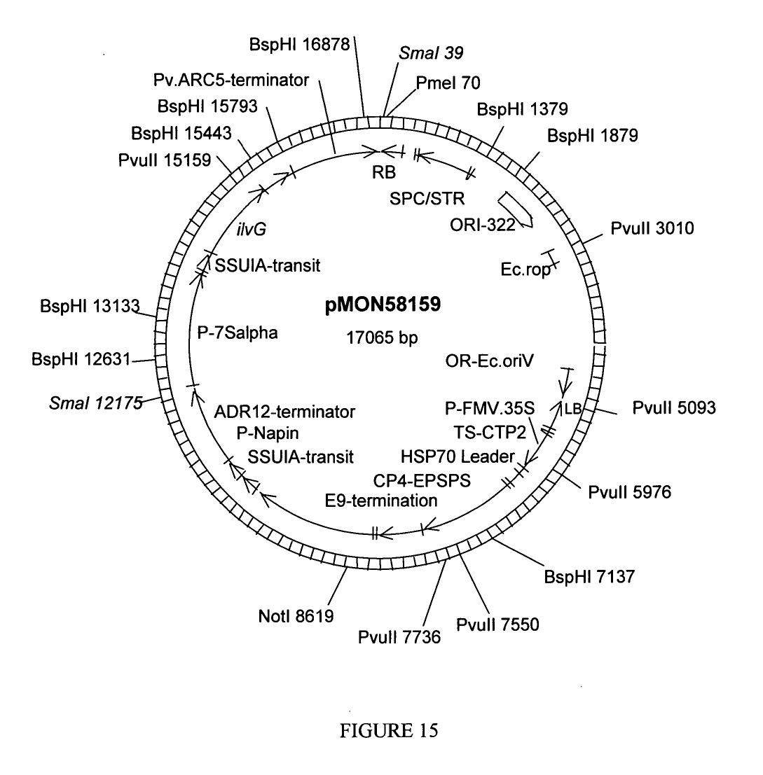

The resulting binary plant transformation plasmids pMON58143 (ilvA466) (FIG. 13), pMON58159 (ilvG+ilvM) (FIG. 14), and pMON58162 (ilvA466+ilvG+ilvM) (FIG. 15), were transformed into Arabidopsis by Agrobacterium mediated infiltration (Beachtold et al., C.R. Acad. Sci. Ser. 111, 316:1194-1199 (1993)). Transformants were selected in the presence of kanamycin.

In order to measure the concentration of amino acids in seeds, 5 mg of mature seed tissue was ground to a fine powder, and the powder extracted in 100 μl of 5% trichloroacetic acid by vortexing at room temperature for 15 minutes. Extracts were centrifuged at 16,000 g for 15 minutes, and the supernatant was transferred to HPLC vials for analysis as described by the manufacturer (Agilent Technologies, USA). Amino acid concentrations were measured by fluorescence spectroscopy at an excitation wavelength of 340 nm and emission of 450 nm.

Several independent events were generated for each construct. Desiccated, mature segregating Arabidopsis seeds were collected as a pool from each event, and subjected to amino acid analysis. The seed from plants transformed with ilvA466 (pMON58143) contained elevated levels of isoleucine showing an approximately 69-fold increase over the average levels of isoleucine found in seeds from plants that were not transformed with ilvA466 (Table 6A). A positive correlation, defined as a Pearson's correlation coefficient (r) of 0.60 or higher (Snedecor and Cochran, In: Statistical Methods, 1980), was observed with other free amino acid concentrations, including arginine, glutamine, leucine, lysine, threonine, tyrosine, phenylalanine, and valine.

The seed from plants transformed with ilvG, ilvM (pMON58159) contained elevated levels of valine that were approximately 15-fold increases over control seed that did not contain ilvG and ilvM, with a positive correlation (r>0.60) for tryptophan, alanine, arginine, glutamine, glycine, serine, phenylalanine, leucine, lysine, threonine, and tyrosine (Table 6B).

The seed from plants transformed with ilvG, ilvM, and ilvA466 (pMON58162) contained elevated levels of isoleucine (15-fold increase) and valine (19-fold increase) with positive correlations (r>0.6) with lysine, phenylalanine, threonine, tyrosine, and valine with respect to isoleucine; and alanine, glutamine, serine, threonine, isoleucine and tyrosine with respect to valine (Table 6C).

TABLE 6A
Amino acid concentrations in Arabidopsis plants expressing the E. coli
ilvA466 allele and correlations with Ile concentrations.

| Amino Acid | Construct | Mean | Std. Dev. | r (Ile) |
|---|---|---|---|---|
| Trp | pMON58143 | 54.1 | 47.4 | 0.377 |
| Ile | pMON58143 | 2624.9 | 625.7 | NA |
| Ala | pMON58143 | 198.4 | 53.3 | 0.538 |
| Arg | pMON58143 | 2364.3 | 727.0 | 0.676 |
| Asn | pMON58143 | 1125.1 | 414.2 | 0.518 |
| Asp | pMON58143 | 234.2 | 55.6 | 0.589 |
| Gln | pMON58143 | 1179.8 | 290.5 | 0.665 |
| Glu | pMON58143 | 841.1 | 158.6 | 0.163 |
| Gly | pMON58143 | 30.8 | 13.5 | 0.406 |
| His | pMON58143 | 335.8 | 207.6 | 0.026 |
| Leu | pMON58143 | 192.0 | 71.5 | 0.925 |
| Lys | pMON58143 | 292.3 | 77.1 | 0.806 |
| Met | pMON58143 | 29.4 | 9.4 | 0.505 |
| Phe | pMON58143 | 100.3 | 20.9 | 0.665 |
| Ser | pMON58143 | 116.8 | 33.3 | 0.217 |
| Thr | pMON58143 | 184.8 | 54.3 | 0.677 |
| Tyr | pMON58143 | 108.2 | 28.6 | 0.627 |
| Val | pMON58143 | 356.3 | 131.1 | 0.829 |

TABLE 6B
Amino acid concentrations in Arabidopsis plants expressing ilvG and
ilvM, and correlations with Ile and Val concentrations.

| Amino Acid | Construct | Mean | Std. Dev. | r (Val) |
|---|---|---|---|---|
| Trp | pMON58159 | 58.7 | 35.4 | 0.867 |
| Ile | pMON58159 | 118.3 | 140.8 | 0.030 |
| Ala | pMON58159 | 196.7 | 87.8 | 0.863 |
| Arg | pMON58159 | 753.7 | 405.8 | 0.771 |
| Asn | pMON58159 | 479.4 | 203.4 | 0.352 |
| Asp | pMON58159 | 178.4 | 54.8 | 0.515 |
| Gln | pMON58159 | 854.7 | 519.6 | 0.979 |
| Glu | pMON58159 | 501.5 | 196.6 | −0.217 |
| Gly | pMON58159 | 42.8 | 16.2 | 0.807 |
| His | pMON58159 | 99.6 | 56.7 | 0.530 |
| Leu | pMON58159 | 239.1 | 160.8 | 0.782 |
| Lys | pMON58159 | 195.9 | 89.4 | 0.920 |
| Met | pMON58159 | 10.2 | 4.5 | −0.448 |
| Phe | pMON58159 | 79.7 | 22.6 | 0.725 |
| Ser | pMON58159 | 968.0 | 608.8 | 0.976 |
| Thr | pMON58159 | 211.9 | 98.9 | 0.932 |
| Tyr | pMON58159 | 94.1 | 48.3 | 0.966 |
| Val | pMON58159 | 2525.3 | 1572.1 | NA |

TABLE 6C
Amino acid concentrations in Arabidopsis plants expressing ilvA466,
ilvG and ilvM, and correlations with Ile and Val concentrations.

| Amino Acid | Construct | Mean | Std. Dev. | r (Ile) | r (Val) |
|---|---|---|---|---|---|
| Trp | pMON58162 | 284.3 | 852.2 | −0.324 | −0.512 |
| Ile | pMON58162 | 566.0 | 299.6 | NA | 0.604 |
| Ala | pMON58162 | 268.9 | 92.2 | 0.468 | 0.697 |
| Arg | pMON58162 | 1723.4 | 859.7 | 0.464 | 0.367 |
| Asn | pMON58162 | 1034.5 | 516.1 | 0.065 | 0.099 |
| Asp | pMON58162 | 261.9 | 127.9 | −0.148 | −0.270 |
| Gln | pMON58162 | 869.4 | 452.5 | 0.578 | 0.764 |
| Glu | pMON58162 | 743.0 | 215.8 | −0.148 | −0.414 |
| Gly | pMON58162 | 34.1 | 13.5 | −0.180 | −0.059 |
| His | pMON58162 | 255.2 | 135.0 | 0.467 | 0.315 |
| Leu | pMON58162 | 451.2 | 377.7 | 0.581 | 0.493 |
| Lys | pMON58162 | 280.2 | 87.5 | 0.662 | 0.585 |
| Met | pMON58162 | 20.3 | 16.2 | 0.204 | −0.157 |
| Phe | pMON58162 | 120.5 | 36.9 | 0.742 | 0.441 |
| Ser | pMON58162 | 486.9 | 319.5 | 0.298 | 0.632 |
| Thr | pMON58162 | 238.1 | 81.8 | 0.708 | 0.825 |
| Tyr | pMON58162 | 127.7 | 43.2 | 0.608 | 0.690 |
| Val | pMON58162 | 3196.3 | 1183.1 | 0.604 | NA |

### EXAMPLE 4

This example sets forth the transformation of soybean plants with expression vectors containing threonine deaminase mutant alleles using particle bombardment and Agrobacterium mediated methods.

Commercially available soybean seeds (Asgrow A3244, A4922) were germinated overnight (approximately 18-24 hours) and the meristem explants were excised. The primary leaves were removed to expose the meristems and the explants were placed in targeting media with the meristems positioned perpendicular to the direction of the particle delivery. Transformation vectors containing the coding regions for the different ilvA alleles pMON53910, pMON53911, and pMON53912 were precipitated onto microscopic gold particles with CaCl₂ and spermidine and subsequently resuspended in ethanol. The suspension was coated onto a Mylar sheet that was then placed onto the electric discharge device. The particles were accelerated into the plant tissue by electric discharge at approximately 60% capacitance.

Following bombardment, the explants were placed in Woody Plant Medium (WPM) (McCown & Lloyd, Proc. International Plant Propagation Soc., 30:421 (1981)) plus 75 mM glyphosate for 5-7 weeks to allow selection and growth of transgenic shoots. Glyphosate positive shoots were harvested approximately 5-7 weeks post-bombardment and placed into selective Bean Rooting Media (BRM) plus 25 mM glyphosate for 2-3 weeks. The composition of BRM is given in Table 7. Shoots producing roots were transferred to the greenhouse and potted in soil. Shoots that remain healthy on selection, but did not produce roots were transferred to non-selective rooting media (bean rooting medium (“BRM”) without glyphosate) for an additional 2 weeks. The roots from any shoots that produced roots off the selection were tested for expression of the glyphosate selectable marker before transferring to the greenhouse and potted in soil. Plants were maintained under standard greenhouse conditions until seed harvest, this seed being defined as the R1 seed.

TABLE 7
Composition and preparation of bean rooting medium (BRM).

| Stock Compounds | Quantity for 4 L | |
|---|---|---|
| MS Salts*** | 8.6 | g |
| Myo-inositol (cell culture grade) | 0.40 | g |
| SBRM Vitamin Stock** | 8.0 | ml |
| L-Cysteine (10 mg/ml) | 40.0 | ml |
| Sucrose (ultra pure) | 120 | g |
| Adjust pH to 5.8 | | |
| Washed Agar | 32 | g |
| Additions after autoclaving: | | |
| SBRM/TSG Hormone Stock* | 20.0 | ml |

*SBRM/TSG Hormone Stock (to 1 L of BRM, add the following)
3.0 ml IAA (0.033 mg/ml)
2.0 ml sterile distilled water
Store stock in dark at 4° C.
**SBRM Vitamin Stock (per 1 L of stock)

| | |
|---|---|
| Glycine | 1.0 g |
| Nicotinic Acid | 0.25 g |
| Pyridoxine HCl | 0.25 g |
| Thiamine HCl | 0.25 g |

***MS Salts (Murashige and Skoog, Physiol. Plant., 15: 473-497 (1962)

This medium is used both with and without the addition of glyphosate (typically 0.025 mM or 0.040 mM). All ingredients are dissolved one at a time. The mixture is brought to volume with sterile distilled water and stored in a foil-covered bottle at 4° C. for no longer than one month.

Soybean plants were also transformed with pMON58028, pMON58029, and pMON58031 using an Agrobacterium-mediated transformation method, as described (Martinell et al., U.S. Pat. No. 6,384,301). For this method, overnight cultures of Agrobacterium tumefaciens containing the plasmid that includes a gene of interest were grown to log phase and then diluted to a final optical density of 0.3 to 0.6 using standard methods known to one skilled in the art. These cultures were used to inoculate the soybean embryo explants prepared as described below.

Briefly, the method is a direct germline transformation into individual soybean cells in the meristem of an excised soybean embryo. The soybean embryo is removed after surface sterilization and germination of the seed. The explants are then plated on OR media, a standard MS medium as modified by Barwale et al., Plants, 167:473-481 (1986), plus 3 mg/L BAP, 200 mg/L Carbenicillin, 62.5 mg/L Cefotaxime, and 60 mg/L Benomyl, and stored at 15° C. overnight in the dark. The following day the explants are wounded with a scalpel blade and inoculated with the Agrobacterium culture prepared as described above. The inoculated explants are then cultured for 3 days at room temperature.

Following the post-transformation culture, the meristemac region is then cultured on standard plant tissue culture media in the presence of the herbicide glyphosate (Monsanto Company, St. Louis, Mo.), which acts as both a selection agent and a shoot inducing hormone. Media compositions and culture lengths are detailed in Martinell et al., U.S. Pat. No. 6,384,301. After 5 to 6 weeks, the surviving explants that have a positive phenotype are transferred to soil and grown under greenhouse conditions until maturity.

The isoleucine concentrations (as described in Example 2) of 5 individual segregating R1 seeds were determined and those events with high concentrations were grown into R1 plants. From each event, 24 seeds were planted. The resulting R2 seed was harvested and isoleucine concentrations were measured, and the presence of the transgene was analyzed. The same analyses were performed for R2 seeds, R2 plants, and R3 seed.

### EXAMPLE 5

This example sets forth the characterization of soybean plants transformed with threonine deaminase gene constructs. To determine threonine deaminase activity, a single seed (˜100 mg) was ground in 100 μL of 1× grind buffer (Table 8). The mixture was then centrifuged for 2-3 minutes at maximum speed. The resulting supematant was desalted by application to a Bio-Rad Bio-Gel P-30 desalting column.

The desalted protein extract (25-50 μL) was added to the 5× assay mixture (Table 8) for a final volume of 100 μL. The mixture was incubated at 37° C. for 30 minutes. The reaction was terminated by adding 100 μL 0.05% dinitrophenyl-hydrazine in 1 N HCl, followed by incubating at room temperature for 10 minutes. An aliquot of 100 μL of 4 N NaOH was then added and the absorbance at 540 nm was measured spectrophotometrically.

TABLE 8
Buffers used in the threonine deaminase enzyme assay.

| | | | 1 X Grind | |
|---|---|---|---|---|
| 5 X Assay Mix - (for 1 mL) | | | Buffer - (for 100 mL) | |
| Component | Aliquot | Concentration | Aliquot | Concentration |
| 2 M Tris-HCl, | 250 μL | (100 mM) | 5 mL | (100 mM) |
| pH 9.0 | | | | |
| 1 M KCl | 500 μL | (100 mM) | 10 mL | (100 mM) |
| 0.5 M L-threonine | 25 μL | (12.5 mM) | 0 | 0 |
| 0.5 mM DTT | | | 4 μL | (2 mM) |
| H₂O | 225 μL | | 85 mL | |

The concentration of free isoleucine in seeds was determined by crushing approximately 50 mg of seed, placing the crushed material in a centrifuge vial, and then weighing. One mL of 5% trichloroacetic acid was added to each sample vial. The samples were mixed, using a vortex mixer, at room temperature for 15 minutes. The samples were then spun in a microcentrifuge for 15 minutes at 14,000 rpm. Some of the supernatant was then removed, placed in a HPLC vial and sealed. Samples were kept at 4° C. prior to analysis.

A single seed analysis was performed on all R1 soybean seed, with 5 seeds per event, and one injection per seed. For subsequent generations representing the R2 and R3 seeds, a bulk assay having 10 seeds for each event, and one injection per event was used.

The samples were analyzed using the Agilent Technologies 1100 series HPLC system. A 0.5 μL aliquot of the sample was derivatized with 2.5 μL of OPA (o-phthalaldehyde and 3-mercaptopropionic acid in borate buffer, Hewlett-Packard PN 5061-3335) reagent in 10 μl of 0.4 N borate buffer pH 10.2 (Hewlett-Packard, PN 5061-3339). The derivative was injected onto an Agilent Technologies Eclipse® XDB-C18 3.5 μm, 4.6×75 mm at 2 mL/min flow rate.

TABLE 9
HPLC experimental conditions.

| | Time (min): | | | | |
|---|---|---|---|---|---|
| | 0 | 9.8 | 12 | 12.5 | 14 |
| % A | 0 | 70 | 0 | 0 | 0 |
| % B | 0 | 30 | 100 | 0 | 0 |

HPLC Buffer A: 95% 40 mM Na₂HPO₄, pH = 7.8 + 5% Buffer B + 0.01% NaN₃
HPLC Buffer B: 45%:45%:10%::Methanol:Acetonitrile:Water.

Isoleucine concentrations were measured using fluorescence detection (excitation at 340 nm, emission at 450 nm) and values were calculated from a standard curve ranging from 10 to 800 μg/mL.

The results for this assessment of free isoleucine concentrations in the transformed soybean plants showed that the free isoleucine concentration for the null control was approximately 100 μg/g in the seed, whereas plants transformed with the ilvA219 and ilvA466 alleles had greater than approximately 600 and 1300 μg/g, respectively. These data indicate that free isoleucine levels are significantly higher in plants transformed with the deregulated threonine deaminase genes as compared to the non-transformed plants.

To determine the presence of threonine deaminase protein in soybean plants transformed with threonine deaminase constructs, mature soybean seeds from lines generated from wild type and isoleucine-deregulated threonine deaminase mutant alleles were subjected to Western blot analysis. Soybean seeds were dried and ground into a powder. To 20 mg of the powder, 200 μl of 1× SDS-PAGE sample buffer was added and the mixture was incubated, with rotation, at 4° C. for 4 hours. The reaction was terminated by boiling for 5-10 minutes. The mixture was then centrifuged for 10 minutes at 14,000 rpm. The resulting supernatant was set aside and the centrifugation was repeated. The combined supernatant fractions were assayed for protein using the Bio-Rad protein assay kit (Bio-Rad).

The supernatant fraction was then separated by SDS-PAGE using a 10% Tris-HCl buffer. After adding a sample dye (10% v/v), 1 mL of the prepared sample was loaded into each sample well. The gel was run at 140 volts for 1 hour in Tris-glycine-SDS buffer. The proteins in the gel were then transferred to a PVDF membrane that had been pre-wetted with methanol and transfer buffer. After loading into the cartridge, the transfer was done at 100 volts for 1 hour in cold Tris-glycine-methanol buffer. The blocking step had been done using a 10% milk solution (5 grams non-fat powdered milk in 50 mL total volume TBS buffer (20 mM Tris, pH 7.5 and 150 mM NaCl) containing 0.1% Tween 20).

The primary antibody was a polyclonal rabbit anti-threonine deaminase antibody, which was diluted at 1:1000 in TBS buffer containing 1% Tween 20, and 1% milk solution. The incubation was run at room temperature for 1 hour or overnight at 4° C. The secondary antibody was a polyclonal anti-rabbit antibody obtained from Sigrna Chemical Co. The developing step was done by washing 3 times for 10 minutes each with TBS containing 1% Tween 20, followed by a 10 minute wash with TBS, and then stained.

The results of the Western blot analysis of R3 seed extracts from transformed soybean plants, at 3 different stages of seed maturity, for a heterozygous line and a null line indicate that the concentration of the mutant protein increases as the seed matures. In the resulting gels the location of the band corresponding to the mutant threonine deaminase protein is visible and the band appears in the lanes corresponding to the transformed plants while being absent in the lanes corresponding to the null lines. Additionally, the intensity of the bands clearly increases as the maturity goes from early to late.

### EXAMPLE 6

This example sets forth the results of the amino acid analyses of R3 soybean seeds transformed with polynucleotide sequences encoding threonine deaminase. Tables 10A-10R provide the statistical means and errors of amino acid concentrations measured for R3 soybean events transformed with threonine deaminase using JMP statistical software (SAS Institute, Cary, N.C., USA). Data are arrayed by zygosity and event.

TABLE 10A
Ile levels in soybean plants expressing threonine deaminase.

| Zygosity | Event | pMON | Gene | N | Mean | Std Dev |
|---|---|---|---|---|---|---|
| Heterozygote | 13894 | 53911 | 7S alpha′-ilvA219 | 12 | 614.6 | 591.0 |
| Heterozygote | 14202 | 53911 | 7S alpha′-ilvA219 | 11 | 380.5 | 331.1 |
| Heterozygote | 14269 | 53912 | 7S alpha′-ilvA466 | 8 | 109.7 | 57.1 |
| Homozygote | 13747 | 53910 | 7S alpha′-ilvA | 3 | 199.6 | 80.4 |
| Homozygote | 14269 | 53912 | 7S alpha′-ilvA466 | 3 | 346.8 | 43.9 |
| Null | 13894 | 53911 | 7S alpha′-ilvA219 | 11 | 37.1 | 20.2 |
| Null | 14202 | 53911 | 7S alpha′-ilvA219 | 10 | 43.4 | 49.3 |
| Null | 14269 | 53912 | 7S alpha′-ilvA466 | 5 | 24.9 | 3.8 |
| Null | A4922 | NA | Base germplasm | 6 | 30.7 | 11.4 |

TABLE 10B
Asp levels in soybean plants expressing threonine deaminase

| Zygosity | Event | pMON | Gene | N | Mean | Std Dev |
|---|---|---|---|---|---|---|
| Heterozygote | 13894 | 53911 | 7S alpha′-ilvA219 | 12 | 148.9 | 28.8 |
| Heterozygote | 14202 | 53911 | 7S alpha′-ilvA219 | 11 | 171.7 | 35.6 |
| Heterozygote | 14269 | 53912 | 7S alpha′-ilvA466 | 8 | 132.6 | 26.1 |
| Homozygote | 13747 | 53910 | 7S alpha′-ilvA | 3 | 98.4 | 12.2 |
| Homozygote | 14269 | 53912 | 7S alpha′-ilvA466 | 3 | 54.2 | 12.7 |
| Null | 13894 | 53911 | 7S alpha′-ilvA219 | 11 | 170.6 | 33 |
| Null | 14202 | 53911 | 7S alpha′-ilvA219 | 10 | 176.3 | 29 |
| Null | 14269 | 53912 | 7S alpha′-ilvA466 | 5 | 144.6 | 36.1 |
| Null | A4922 | NA | Base germplasm | 6 | 178.8 | 19.7 |

TABLE 10C
Glu levels in soybean plants expressing threonine deaminase

| Zygosity | Event | pMON | Gene | N | Mean | Std Dev |
|---|---|---|---|---|---|---|
| Heterozygote | 13894 | 53911 | 7S alpha′-ilvA219 | 12 | 213.0 | 40.4 |
| Heterozygote | 14202 | 53911 | 7S alpha′-ilvA219 | 11 | 225.0 | 40.3 |
| Heterozygote | 14269 | 53912 | 7S alpha′-ilvA466 | 8 | 224.9 | 36.0 |
| Homozygote | 13747 | 53910 | 7S alpha′-ilvA | 3 | 338.9 | 20.2 |
| Homozygote | 14269 | 53912 | 7S alpha′-ilvA466 | 3 | 194.5 | 31.8 |
| Null | 13894 | 53911 | 7S alpha′-ilvA219 | 11 | 210.1 | 38.3 |
| Null | 14202 | 53911 | 7S alpha′-ilvA219 | 10 | 225.9 | 42.6 |
| Null | 14269 | 53912 | 7S alpha′-ilvA466 | 5 | 224.2 | 22.8 |
| Null | A4922 | NA | Base germplasm | 6 | 236.2 | 21.5 |

TABLE 10D
Asn levels in soybean plants expressing threonine deaminase

| Zygosity | Event | pMON | Gene | N | Mean | Std Dev |
|---|---|---|---|---|---|---|
| Heterozygote | 13894 | 53911 | 7S alpha′-ilvA219 | 12 | 29.6 | 9.9 |
| Heterozygote | 14202 | 53911 | 7S alpha′-ilvA219 | 11 | 28.3 | 9.4 |
| Heterozygote | 14269 | 53912 | 7S alpha′-ilvA466 | 8 | 24.5 | 8.7 |
| Homozygote | 13747 | 53910 | 7S alpha′-ilvA | 3 | 230.3 | 151.8 |
| Homozygote | 14269 | 53912 | 7S alpha′-ilvA466 | 3 | 25.3 | 9.9 |
| Null | 13894 | 53911 | 7S alpha′-ilvA219 | 11 | 23.8 | 7.0 |
| Null | 14202 | 53911 | 7S alpha′-ilvA219 | 10 | 22.9 | 4.2 |
| Null | 14269 | 53912 | 7S alpha′-ilvA466 | 5 | 25.0 | 10.3 |
| Null | A4922 | NA | Base germplasm | 6 | 24.3 | 3.3 |

TABLE 10E
Ser levels in soybean plants expressing threonine deaminase

| Zygosity | Event | pMON | Gene | N | Mean | Std Dev |
|---|---|---|---|---|---|---|
| Heterozygote | 13894 | 53911 | 7S alpha′-ilvA219 | 12 | 17.8 | 5.7 |
| Heterozygote | 14202 | 53911 | 7S alpha′-ilvA219 | 11 | 16.2 | 3.6 |
| Heterozygote | 14269 | 53912 | 7S alpha′-ilvA466 | 8 | 14.4 | 2.0 |
| Homozygote | 13747 | 53910 | 7S alpha′-ilvA | 3 | 13.8 | 3.6 |
| Homozygote | 14269 | 53912 | 7S alpha′-ilvA466 | 3 | 13.4 | 3.3 |
| Null | 13894 | 53911 | 7S alpha′-ilvA219 | 11 | 14.7 | 3.4 |
| Null | 14202 | 53911 | 7S alpha′-ilvA219 | 10 | 14.3 | 1.7 |
| Null | 14269 | 53912 | 75 alpha′-ilvA466 | 5 | 14.0 | 1.4 |
| Null | A4922 | NA | Base germplasm | 6 | 15.0 | 1.5 |

TABLE 10F
Gln levels in soybean plants expressing threonine deaminase

| Zygosity | Event | pMON | Gene | N | Mean | Std Dev |
|---|---|---|---|---|---|---|
| Heterozygote | 13894 | 53911 | 7S alpha′-ilvA219 | 12 | 4.8 | 1.5 |
| Heterozygote | 14202 | 53911 | 7S alpha′-ilvA219 | 11 | 4.5 | 0.8 |
| Heterozygote | 14269 | 53912 | 7S alpha′-ilvA466 | 8 | 4.9 | 1.1 |
| Homozygote | 13747 | 53910 | 7S alpha′-ilvA | 3 | 34.7 | 41.9 |
| Homozygote | 14269 | 53912 | 7S alpha′-ilvA466 | 3 | 4.7 | 0.3 |
| Null | 13894 | 53911 | 7S alpha′-ilvA219 | 11 | 4.1 | 1.3 |
| Null | 14202 | 53911 | 7S alpha′-ilvA219 | 10 | 4.0 | 0.6 |
| Null | 14269 | 53912 | 7S alpha′-ilvA466 | 5 | 4.6 | 0.6 |
| Null | A4922 | NA | Base germplasm | 6 | 4.3 | 0.4 |

TABLE 10G
His levels in soybean plants expressing threonine deaminase

| Zygosity | Event | pMON | Gene | N | Mean | Std Dev |
|---|---|---|---|---|---|---|
| Heterozygote | 13894 | 53911 | 7S alpha′-ilvA219 | 12 | 40.3 | 33.2 |
| Heterozygote | 14202 | 53911 | 7S alpha′-ilvA219 | 11 | 28.0 | 19.5 |
| Heterozygote | 14269 | 53912 | 7S alpha′-ilvA466 | 8 | 18.0 | 7.9 |
| Homozygote | 13747 | 53910 | 7S alpha′-ilvA | 3 | 48.1 | 22.5 |
| Homozygote | 14269 | 53912 | 7S alpha′-ilvA466 | 3 | 20.2 | 9.1 |
| Null | 13894 | 53911 | 7S alpha′-ilvA219 | 11 | 12.8 | 3.3 |
| Null | 14202 | 53911 | 7S alpha′-ilvA219 | 10 | 12.6 | 3.8 |
| Null | 14269 | 53912 | 7S alpha′-ilvA466 | 5 | 14.3 | 5.0 |
| Null | A4922 | NA | Base germplasm | 6 | 13.5 | 1.9 |

TABLE 10H
Gly levels in soybean plants expressing threonine deaminase

| Zygosity | Event | pMON | Gene | N | Mean | Std Dev |
|---|---|---|---|---|---|---|
| Heterozygote | 13894 | 53911 | 7S alpha′-ilvA219 | 12 | 17.6 | 5.3 |
| Heterozygote | 14202 | 53911 | 7S alpha′-ilvA219 | 11 | 17.8 | 4.7 |
| Heterozygote | 14269 | 53912 | 7S alpha′-ilvA466 | 8 | 15.8 | 2.7 |
| Homozygote | 13747 | 53910 | 7S alpha′-ilvA | 3 | 154.8 | 87.6 |
| Homozygote | 14269 | 53912 | 7S alpha′-ilvA466 | 3 | 18.8 | 1.7 |
| Null | 13894 | 53911 | 7S alpha′-ilvA219 | 11 | 14.1 | 3.6 |
| Null | 14202 | 53911 | 7S alpha′-ilvA219 | 10 | 16.8 | 8.7 |
| Null | 14269 | 53912 | 7S alpha′-ilvA466 | 5 | 14.6 | 1.5 |
| Null | A4922 | NA | Base germplasm | 6 | 14.7 | 1.2 |

TABLE 10I
Thr levels in soybean plants expressing threonine deaminase

| Zygosity | Event | pMON | Gene | N | Mean | Std Dev |
|---|---|---|---|---|---|---|
| Heterozygote | 13894 | 53911 | 7S alpha′-ilvA219 | 12 | 10.9 | 2.6 |
| Heterozygote | 14202 | 53911 | 7S alpha′-ilvA219 | 11 | 10.8 | 1.9 |
| Heterozygote | 14269 | 53912 | 7S alpha′-ilvA466 | 8 | 9.4 | 1.2 |
| Homozygote | 13747 | 53910 | 7S alpha′-ilvA | 3 | 6.3 | 2.0 |
| Homozygote | 14269 | 53912 | 7S alpha′-ilvA466 | 3 | 5.4 | 0.7 |
| Null | 13894 | 53911 | 7S alpha′-ilvA219 | 11 | 10.6 | 1.6 |
| Null | 14202 | 53911 | 7S alpha′-ilvA219 | 10 | 10.3 | 1.2 |
| Null | 14269 | 53912 | 7S alpha′-ilvA466 | 5 | 10.0 | 1.0 |
| Null | A4922 | NA | Base germplasm | 6 | 10.8 | 0.5 |

TABLE 10J
Arg levels in soybean plants expressing threonine deaminase

| Zygosity | Event | pMON | Gene | N | Mean | Std Dev |
|---|---|---|---|---|---|---|
| Heterozygote | 13894 | 53911 | 7S alpha′-ilvA219 | 12 | 308.3 | 121.9 |
| Heterozygote | 14202 | 53911 | 7S alpha′-ilvA219 | 11 | 299.3 | 126.2 |
| Heterozygote | 14269 | 53912 | 7S alpha′-ilvA466 | 8 | 302.1 | 134.5 |
| Homozygote | 13747 | 53910 | 7S alpha′-ilvA | 3 | 475.6 | 188.1 |
| Homozygote | 14269 | 53912 | 7S alpha′-ilvA466 | 3 | 352.9 | 119.9 |
| Null | 13894 | 53911 | 7S alpha′-ilvA219 | 11 | 216.5 | 49.8 |
| Null | 14202 | 53911 | 7S alpha′-ilvA219 | 10 | 215.1 | 54.9 |
| Null | 14269 | 53912 | 7S alpha′-ilvA466 | 5 | 228.0 | 61.6 |
| Null | A4922 | NA | Base germplasm | 6 | 190.9 | 29.3 |

TABLE 10K
Ala levels in soybean plants expressing threonine deaminase

| Zygosity | Event | pMON | Gene | N | Mean | Std Dev |
|---|---|---|---|---|---|---|
| Heterozygote | 13894 | 53911 | 7S alpha′-ilvA219 | 12 | 56.1 | 13.5 |
| Heterozygote | 14202 | 53911 | 7S alpha′-ilvA219 | 11 | 64.9 | 8.8 |
| Heterozygote | 14269 | 53912 | 7S alpha′-ilvA466 | 8 | 69.5 | 7.9 |
| Homozygote | 13747 | 53910 | 7S alpha′-ilvA | 3 | 82.0 | 44.5 |
| Homozygote | 14269 | 53912 | 7S alpha′-ilvA466 | 3 | 60.2 | 7.8 |
| Null | 13894 | 53911 | 7S alpha′-ilvA219 | 11 | 65.7 | 11.6 |
| Null | 14202 | 53911 | 7S alpha′-ilvA219 | 10 | 70.9 | 8.4 |
| Null | 14269 | 53912 | 7S alpha′-ilvA466 | 5 | 76.2 | 8.3 |
| Null | A4922 | NA | Base germplasm | 6 | 72.8 | 8.7 |

TABLE 10L
Tyr levels in soybean plants expressing threonine deaminase

| Zygosity | Event | pMON | Gene | N | Mean | Std Dev |
|---|---|---|---|---|---|---|
| Heterozygote | 13894 | 53911 | 7S alpha′-ilvA219 | 12 | 15.5 | 53.6 |
| Heterozygote | 14202 | 53911 | 7S alpha′-ilvA219 | 11 | 25.3 | 83.8 |
| Heterozygote | 14269 | 53912 | 7S alpha′-ilvA466 | 8 | 0.0 | 0.0 |
| Homozygote | 13747 | 53910 | 7S alpha′-ilvA | 3 | 0.0 | 0.0 |
| Homozygote | 14269 | 53912 | 7S alpha′-ilvA466 | 3 | 71.0 | 123.0 |
| Null | 13894 | 53911 | 7S alpha′-ilvA219 | 11 | 22.9 | 75.9 |
| Null | 14202 | 53911 | 7S alpha′-ilvA219 | 10 | 18.3 | 57.8 |
| Null | 14269 | 53912 | 7S alpha′-ilvA466 | 5 | 41.1 | 91.9 |
| Null | A4922 | NA | Base germplasm | 6 | 75.4 | 116.8 |

TABLE 10M
Val levels in soybean plants expressing threonine deaminase

| Zygosity | Event | pMON | Gene | N | Mean | Std Dev |
|---|---|---|---|---|---|---|
| Heterozygote | 13894 | 53911 | 7S alpha′-ilvA219 | 12 | 51.2 | 30.9 |
| Heterozygote | 14202 | 53911 | 7S alpha′-ilvA219 | 11 | 39.3 | 12.2 |
| Heterozygote | 14269 | 53912 | 7S alpha′-ilvA466 | 8 | 27.0 | 5.6 |
| Homozygote | 13747 | 53910 | 7S alpha′-ilvA | 3 | 38.3 | 14.6 |
| Homozygote | 14269 | 53912 | 7S alpha′-ilvA466 | 3 | 27.6 | 5.8 |
| Null | 13894 | 53911 | 7S alpha′-ilvA219 | 11 | 31.5 | 4.7 |
| Null | 14202 | 53911 | 7S alpha′-ilvA219 | 10 | 31.8 | 5.0 |
| Null | 14269 | 53912 | 7S alpha′-ilvA466 | 5 | 26.1 | 6.3 |
| Null | A4922 | NA | Base germplasm | 6 | 31.3 | 6.3 |

TABLE 10N
Met levels in soybean plants expressing threonine deaminase

| Zygosity | Event | pMON | Gene | N | Mean | Std Dev |
|---|---|---|---|---|---|---|
| Heterozygote | 13894 | 53911 | 7S alpha′-ilvA219 | 12 | 13.7 | 3.8 |
| Heterozygote | 14202 | 53911 | 7S alpha′-ilvA219 | 11 | 13.3 | 4.6 |
| Heterozygote | 14269 | 53912 | 7S alpha′-ilvA466 | 8 | 12.6 | 3.9 |
| Homozygote | 13747 | 53910 | 7S alpha′-ilvA | 3 | 35.0 | 10.9 |
| Homozygote | 14269 | 53912 | 7S alpha′-ilvA466 | 3 | 21.3 | 4.4 |
| Null | 13894 | 53911 | 7S alpha′-ilvA219 | 11 | 9.4 | 1.7 |
| Null | 14202 | 53911 | 7S alpha′-ilvA219 | 10 | 9.7 | 1.7 |
| Null | 14269 | 53912 | 7S alpha′-ilvA466 | 5 | 9.1 | 1.6 |
| Null | A4922 | NA | Base germplasm | 6 | 10.0 | 0.9 |

TABLE 10O
Trp levels in soybean plants expressing threonine deaminase

| Zygosity | Event | pMON | Gene | N | Mean | Std Dev |
|---|---|---|---|---|---|---|
| Heterozygote | 13894 | 53911 | 7S alpha′-ilvA219 | 12 | 172.7 | 38.1 |
| Heterozygote | 14202 | 53911 | 7S alpha′-ilvA219 | 11 | 165.6 | 42.5 |
| Heterozygote | 14269 | 53912 | 7S alpha′-ilvA466 | 8 | 166.6 | 38.2 |
| Homozygote | 13747 | 53910 | 7S alpha′-ilvA | 3 | 152.5 | 75.4 |
| Homozygote | 14269 | 53912 | 7S alpha′-ilvA466 | 3 | 163.9 | 4.3 |
| Null | 13894 | 53911 | 7S alpha′-ilvA219 | 11 | 127.6 | 20.7 |
| Null | 14202 | 53911 | 7S alpha′-ilvA219 | 10 | 121.0 | 18.0 |
| Null | 14269 | 53912 | 7S alpha′-ilvA466 | 5 | 131.7 | 12.6 |
| Null | A4922 | NA | Base germplasm | 6 | 130.2 | 10.0 |

TABLE 10P
Phe levels in soybean plants expressing threonine deaminase

| Zygosity | Event | pMON | Gene | N | Mean | Std Dev |
|---|---|---|---|---|---|---|
| Heterozygote | 13894 | 53911 | 7S alpha′-ilvA219 | 12 | 26.2 | 8.5 |
| Heterozygote | 14202 | 53911 | 7S alpha′-ilvA219 | 11 | 25.1 | 9.0 |
| Heterozygote | 14269 | 53912 | 7S alpha′-ilvA466 | 8 | 20.2 | 4.7 |
| Homozygote | 13747 | 53910 | 7S alpha′-ilvA | 3 | 16.7 | 2.5 |
| Homozygote | 14269 | 53912 | 7S alpha′-ilvA466 | 3 | 29.1 | 0.4 |
| Null | 13894 | 53911 | 7S alpha′-ilvA219 | 11 | 17.1 | 2.6 |
| Null | 14202 | 53911 | 7S alpha′-ilvA219 | 10 | 17.8 | 2.6 |
| Null | 14269 | 53912 | 7S alpha′-ilvA466 | 5 | 16.7 | 2.9 |
| Null | A4922 | NA | Base germplasm | 6 | 17.9 | 1.4 |

TABLE 10Q
Leu levels in soybean plants expressing threonine deaminase

| Zygosity | Event | pMON | Gene | N | Mean | Std Dev |
|---|---|---|---|---|---|---|
| Heterozygote | 13894 | 53911 | 7S alpha′-ilvA219 | 12 | 30.2 | 15.7 |
| Heterozygote | 14202 | 53911 | 7S alpha′-ilvA219 | 11 | 24.7 | 11.2 |
| Heterozygote | 14269 | 53912 | 7S alpha′-ilvA466 | 8 | 18.1 | 4.9 |
| Homozygote | 13747 | 53910 | 7S alpha′-ilvA | 3 | 39.0 | 11.4 |
| Homozygote | 14269 | 53912 | 7S alpha′-ilvA466 | 3 | 32.5 | 2.6 |
| Null | 13894 | 53911 | 7S alpha′-ilvA219 | 11 | 12.8 | 1.9 |
| Null | 14202 | 53911 | 7S alpha′-ilvA219 | 10 | 13.1 | 2.5 |
| Null | 14269 | 53912 | 7S alpha′-ilvA466 | 5 | 12.5 | 1.6 |
| Null | A4922 | NA | Base germplasm | 6 | 12.6 | 1.2 |

TABLE 10R
Lys levels in soybean plants expressing threonine deaminase

| Zygosity | Event | pMON | Gene | N | Mean | Std Dev |
|---|---|---|---|---|---|---|
| Heterozygote | 13894 | 53911 | 7S alpha′-ilvA219 | 12 | 62.7 | 47.5 |
| Heterozygote | 14202 | 53911 | 7S alpha′-ilvA219 | 11 | 36.5 | 22.0 |
| Heterozygote | 14269 | 53912 | 7S alpha′-ilvA466 | 8 | 24.6 | 11.6 |
| Homozygote | 13747 | 53910 | 7S alpha′-ilvA | 3 | 14.9 | 2.8 |
| Homozygote | 14269 | 53912 | 7S alpha′-ilvA466 | 3 | 47.8 | 27.6 |
| Null | 13894 | 53911 | 7S alpha′-ilvA219 | 11 | 17.7 | 3.8 |
| Null | 14202 | 53911 | 7S alpha′-ilvA219 | 10 | 20.9 | 7.6 |
| Null | 14269 | 53912 | 7S alpha′-ilvA466 | 5 | 18.8 | 2.7 |
| Null | A4922 | NA | Base germplasm | 6 | 19.7 | 3.1 |

The results of the amino acid analysis presented in Tables 10A through 10R show that the concentration of a number of amino acids increases in soybean plants transformed with polynucleotide sequences encoding threonine deaminase. Data are segregated by zygosity. A pooled estimate, which removes the effect of zygosity, is also provided. The data were subjected to correlation analysis using the method of Pearson (Snedecor and Cochran, In: Statistical Methods, 1982; JMP statistical software (SAS Institute, Cary, N.C., USA). Numerical values represent Pearson's correlation coefficient (r). Positive values of 0.60 or higher show a positive correlation in the concentration of an amino acid with the concentration of Ile. In the heterozygous condition the amino acids Asn, Ser, His, Gly, Thr, Arg, Val, Met, Phe, Leu, and Lys, were positively correlated with Ile levels. In the homozygous condition, Phe and Lys were positively correlated with Ile concentration.

TABLE 11
Correlation of Ile concentration with other amino acids.

| Amino Acid | Heterzygous | Homozygous | Null | Pooled |
|---|---|---|---|---|
| Asp | 0.0980 | −0.8622 | 0.4567 | −0.0809 |
| Glu | −0.0054 | −0.7400 | 0.4711 | −0.0134 |
| Asn | 0.6754 | −0.4476 | 0.3528 | 0.0925 |
| Ser | 0.7795 | 0.3465 | 0.2511 | 0.6755 |
| Gln | 0.3713 | −0.4478 | 0.3311 | 0.0297 |
| His | 0.9667 | −0.1998 | 0.5769 | 0.9074 |
| Gly | 0.7912 | −0.4385 | 0.1686 | 0.0812 |
| Thr | 0.6827 | 0.1135 | 0.2806 | 0.3667 |
| Arg | 0.6686 | 0.1362 | 0.4715 | 0.6214 |
| Ala | −0.0320 | −0.1327 | 0.3596 | −0.1400 |
| Tyr | 0.1155 | 0.1180 | 0.0082 | −0.0047 |
| Val | 0.9561 | −0.1752 | 0.4279 | 0.8940 |
| Met | 0.8940 | −0.2349 | 0.6515 | 0.4118 |
| Trp | 0.5249 | 0.4908 | 0.2077 | 0.6049 |
| Phe | 0.8488 | 0.8805 | 0.5792 | 0.8489 |
| Leu | 0.9703 | 0.0609 | 0.7700 | 0.8825 |
| Lys | 0.8287 | 0.6265 | 0.8070 | 0.8439 |

All publications and patents are incorporated by reference herein, as though individually incorporated by reference. The present invention is not limited to the exact details shown and described, for it should be understood that many variations and modifications may be made while remaining within the spirit and scope of the present invention defined by the statements.

1. A DNA construct comprising multiple plant expression cassettes wherein a first expression cassette comprises a promoter functional in cells of a plant operably linked to an exogenous polynucleotide encoding a feedback insensitive threonine deaminase and a second expression cassette comprises a promoter functional in cells of a plant operably linked to an exogenous polynucleotide encoding AHAS.

2. A DNA construct comprising multiple plant expression cassettes wherein a first expression cassette comprises a promoter functional in cells of a plant operably linked to an exogenous polynucleotide encoding a feedback insensitive threonine deaminase and a second expression cassette comprises a large subunit of AHAS and a third expression cassette comprises a promoter functional in cells of a plant operably linked to an exogenous polynucleotide encoding a small subunit of AHAS.

3. The DNA construct of claim 1 or 2, wherein each of said promoters is a seed enhanced promoter.

4. The DNA construct of claim 1 or 2, wherein each of said promoters is selected from the group consisting of: napin, 7S alpha, 7S alpha′, 7S beta, USP 88, enhanced USP 88, Arcelin 5, and Oleosin.

5. The DNA construct of claim 3, wherein there are at least two different seed enhanced promoters.

6. The DNA construct of claim 1 or 2, wherein said first cassette comprises a polynucleotide encoding a feedback insensitive threonine deaminase comprising SEQ ID NO: 22.

7. The DNA construct of claim 1 or 2, wherein said first cassette comprises an exogenous polynucleotide encoding a threonine deaminase variant allele or subunit thereof comprising an amino acid substitution at position L447F, or L481F, or L481Y, or L481P, or L481E, or L481T, or L481Q, or L481I, or L481V, or L481M, or L481K.

8. The DNA construct of claim 1 or 2, wherein said polynucleotide encoding a threonine deaminase variant allele is SEQ ID NO: 2 comprising an amino acid substitution at position L447F, or L481F, or L481Y, or L481P, or L481E, or L481T, or L481Q, or L481I, or L481V or L481M, or L481K.

9. The DNA construct of claim 1 or 2, wherein said first cassette further comprises a polynucleotide encoding a plastid transit peptide operably linked to polynucleotide encoding said threonine deaminase.

10. The DNA construct of claim 2, wherein said second expression cassette comprises a polynucleotide encoding the large subunit of AHAS.

11. The DNA construct of claim 10, wherein the polynucleotide encoding the large subunit of AHAS comprises SEQ ID NO: 16.

12. The DNA construct of claim 10, wherein a polynucleotide encoding a plastid transit peptide is operably linked to said polynucleotide encoding said large subunit of AHAS.

13. The DNA construct of claim 2, wherein said third expression cassette comprises a polynucleotide encoding the small subunit of AHAS.

14. The DNA construct of claim 13, wherein the polynucleotide encoding the small subunit of AHAS comprises of SEQ ID NO: 17.

15. The DNA construct of claim 13, wherein a polynucleotide encoding a plastid transit peptide is operably linked to said polynucleotide encoding said small subunit of AHAS.

16. A DNA construct comprising multiple plant expression cassettes wherein a first expression cassette comprises a promoter functional in cells of a plant operably linked to an exogenous polynucleotide encoding a feedback insensitive threonine deaminase, and a second expression cassette comprises a promoter functional in cells of a plant operably linked to an exogenous polynucleotide encoding a large subunit of AHAS.

17. The DNA construct of claim 16, wherein each of said promoters is a seed enhanced promoter.

18. The DNA construct of claim 17, wherein each of said seed enhanced promoters is selected from the group consisting of: napin, 7S alpha, 7S alpha′, 7S beta, USP 88, enhanced USP 88, Arcelin 5, and Oleosin.

19. The DNA construct of claim 16 or 17, wherein there are at least two different seed enhanced promoters.

20. The DNA construct of claim 16, wherein said first cassette comprises a polynucleotide encoding a feedback insensitive threonine deaminase comprising SEQ ID NO: 22.

21. The DNA construct of claim 16, wherein said first cassette comprises a threonine deaminase variant allele comprising an amino acid substitution at position L447F, or L481F, or L481Y, or L481P, or L481E, or L481T, or L481Q, or L481I, or L481V, or L481M, or L481K.

22. The DNA construct of claim 16, wherein said polynucleotide encoding a threonine deaminase variant allele is SEQ ID NO: 2 comprising an amino acid substitution at position L447F, or L481F, or L481Y, or L481P, or L481E, or L481T, or L481Q, or L481I, or L481V, or L481M, or L481K.

23. The DNA construct of claim 16, wherein said first cassette comprises a polynucleotide encoding a plastid transit peptide operably linked to said polynucleotide encoding a threonine deaminase.

24. The DNA construct of claim 16, wherein said second expression cassette comprises a polynucleotide encoding the large subunit of AHAS.

25. The DNA construct of claim 24, wherein the polynucleotide encoding the large subunit of AHAS comprises SEQ ID NO: 16.

26. The DNA construct of claim 25, wherein a polynucleotide encoding a plastid transit peptide is operably linked to said polynucleotide encoding said large subunit of AHAS.

27. A DNA construct comprising multiple plant expression cassettes wherein an expression cassette comprising a promoter functional in cells of a plant is operably linked to an exogenous polynucleotide encoding a monomeric AHAS.

28. A DNA construct comprising multiple plant expression cassettes wherein a first expression cassette comprising a promoter functional in cells of a plant is operably linked to an exogenous polynucleotide encoding a large subunit of AHAS, and a second expression cassette comprising a promoter functional in cells of a plant is operably linked to an exogenous polynucleotide encoding a small subunit of AHAS.

29. The DNA construct of claim 28, wherein each of said promoters is a seed enhanced promoter.

30. The DNA construct of claim 28, wherein each of said seed enhanced promoters is selected from the group consisting of: napin, 7S alpha, 7S alpha′, 7S beta, USP 88, enhanced USP 88, Arcelin 5, and Oleosin.

31. The DNA construct of claim 28, wherein there are at least two different seed enhanced promoters.

32. The DNA construct of claim 28, wherein said first cassette comprises a large subunit of AHAS consisting of SEQ ID NO: 16.

33. The DNA construct of claim 29, wherein said first cassette comprises a polynucleotide encoding a plastid transit peptide operably linked to said polynucleotide encoding said large subunit of AHAS.

34. The DNA construct of claim 28, wherein said second cassette comprises a polynucleotide encoding the small subunit of AHAS.

35. The DNA construct of claim 28, wherein said second cassette comprises a polynucleotide encoding the small subunit of AHAS consisting of SEQ ID NO: 17.

36. The DNA construct of claim 35, wherein said second cassette comprises a polynucleotide encoding a plastid transit peptide operably linked to said polynucleotide encoding said small subunit of AHAS.

37. A method for preparing a transgenic dicot plant having an increase in amino acid level in the seed as compared to a seed from a non-transgenic plant of the same plant species, comprising the steps of: a) introducing into regenerable cells of a dicot plant a transgene comprising the construct of claim 1 or 2; b) regenerating said regenerable cell into a dicot plant; c) harvesting seed from said plant; d) selecting one or more seeds with an increased level of amino acid as compared to a seed from a non-trangenic plant of the same plant species; and e) planting said seed, wherein, if isoleucine is present at an increased level, at least one additional level of amino acid is also increased.

38. The method of claim 37, wherein the dicot plant is a soybean plant.

39. The method of claim 37, wherein the increased level of amino acids comprises an increase in the concentration of: a) Ile and one or more of Arg, Asn, Asp, His, Met, Ala, Leu, Thr, Val, Gln, Tyr, Lys, Ser, and Phe or b) one or more of Arg, Asn, Asp, His, Met, Leu, Val, Gln, Tyr, Thr, Lys, Ala, Ser, and Phe.

40. A transgenic soybean plant produced by the method of claim 37.

41. A method for preparing a transgenic dicot plant having an increased amino acid content in the seed as compared to a seed from a non-transgenic plant of the same plant species, comprising the steps of: a) introducing into regenerable cells of a dicot plant a transgene comprising the construct of claim 16; b) regenerating said regenerable cell into a dicot plant; c) harvesting seed from said plant; d) selecting one or more seeds with an increased level of amino acid as compared to a seed from a non-transgenic plant of the same plant species; and e) planting said seed, wherein, if isoleucine is present at an increased level, at least one additional level of amino acid is also increased.

42. The method of claim 41, wherein the dicot plant is a soybean plant or canola plant.

43. The method of claim 41, wherein the increased level of amino acids comprises an increase in the concentration of: a) Ile and one or more of Arg, Asn, Asp, His, Met, Ala, Leu, Thr, Val, Gln, Tyr, Lys, Ser, and Phe or b) one or more of Arg, Asn, Asp, His, Met, Leu, Val, Gln, Tyr, Thr, Lys, Ala, Ser, and Phe.

44. A transgenic soybean plant produced by the method of claim 41.

45. A method for preparing a transgenic dicot plant having an increased amino acid content in the seed as compared to a seed from a non-transgenic plant of the same plant species, comprising the steps of: a) introducing into regenerable cells of a dicot plant a transgene comprising the construct of claim 27 or 28; b) regenerating said regenerable cell into a dicot plant; c) harvesting seed from said plant; d) selecting one or more seeds with an increased level of amino acid as compared to a seed from a non-trangenic plant of the same plant species; and e) planting said seed.

46. The method of claim 45, wherein the dicot plant is a soybean plant or canola plant.

47. The method of claim 45, wherein the increased level of amino acids comprises an increase in the concentration of Ser or Val.

48. A transgenic soybean plant produced by the method of claim 45.

49. Meal produced from the soybean of claim 40, 44, or 48.
